(19) United States
(12) Patent Application Publication
(10) Pub. No.: US 2004/0110930 A1
(43) Pub. Date: Jun. 10, 2004

(54) Multimeric protein engineering

(76) Inventors: Stephen J. Reinl, Sacramento, CA (US); Patricia C. Edwards, Sacramento, CA (US)

Correspondence Address: Michelle L. Samonek, McDonough Holland & Allen PC, 9th Floor, 555 Capitol Mall, Sacramento, CA, 95814, US

(21) Appl. No.: 10/679,620

(22) Filed: Oct. 3, 2003

Related U.S. Application Data
(60) Provisional application No. 60/415,940, filed on Oct. 3, 2002.

(51) Int. Cl. C07K 16/46; C12Q 1/68; C07H 21/04; C12P 21/04; C12N 5/06

(52) U.S. Cl. 530/387.1; 536/23.53; 435/69.7; 435/320.1; 435/326

(57) ABSTRACT

The invention described herein encompasses (1) artificial preproproteins and the polynucleotides encoding them, (2) methods for producing these biomolecules, and (3) methods for their use. The artificial preproproteins of this invention comprise a protein assembly capable of producing a multimeric protein from a single protein. FIG. 4 illustrates generally the process by which a polynucleotide encoding the artificial preproprotein is introduced into a cell and a biomolecule of interest is produced.

### CROSS REFERENCE TO RELATED APPLICATIONS

This application claims the benefit under 35 U.S.C. §119(e) of U.S. Provisional Application No. 60/415,940, filed Oct. 3, 2003. The content of this application is hereby incorporated by reference into the present disclosure.

### TECHNICAL FIELD

The present invention relates to the expression and assembly of artificial multimeric proteins, i.e. antibodies and antibody fragments, in eukaryotes, i.e. plants.

### BACKGROUND

It is known that polypeptides can be expressed in a wide variety of cellular hosts. A wide variety of genes have been isolated from mammals and viruses, joined to transcriptional and translational initiation and termination regulatory signals from a heterologous source, and introduced into hosts into which these regulatory signals are functional.

Plants are an important system for the expression of many recombinant proteins, especially those intended for therapeutic purposes. Heterologous proteins are reliably made in one of two general ways, either by nuclear transformation of the chromosomal DNA or by infecting the host with viral vector. Transgenic plants are created by the stable integration of the foreign DNA into the plant genome, and subsequent genetic recombination by crossing of transgenic plants is a simple method for introducing new genes and accumulating multiple genes into plants. Alternatively, viral vectors engineered to carry heterologous genes can be used to transfect the host such that the genes are carried in an episomal manner, parasitizing the host translational machinery to produce the protein of interest. Regardless of how the delivery of the foreign genes is accomplished, plants are attractive hosts because of the opportunity for protein production on an agricultural scale at an extremely competitive cost, but there are also many other advantages. The processing and assembly of recombinant proteins in plants may also complement that in mammalian cells, which may be an advantage over the more commonly used microbial expression systems.

One of the most useful aspects of using a recombinant expression system for antibody production is the ease with which the antibody can be tailored by molecular engineering. This allows the production of antibody fragments, as well as the manipulation of full-length antibodies. For example, a side range of functional recombinant-antibody fragments, such as Fab's, may be generated. In addition, the ability of plant cells to produce full-length antibodies can be exploited for the production of antibody molecules with altered Fc-mediated properties. This is facilitated by the domain structure of immunoglobulin chains, which allows individual domains to be “cut and spliced” at the gene level. For example, substituting the Fc region of an IgM with that of an IgG, while maintaining the correct assembly of the functional antibody in plants. These alterations have no effect on antigen binding or specificity, but may modify the protective functions of the antibody that are mediated through the Fc region.

The immunoglobulin molecule is composed of two identical heavy chains and two identical light chains (H₂L₂) where the two chains are present in equimolar ratio and are linked by a disulfide bond. The diversity of antibodies created through multiple genes encoding the heavy and light chains, rearrangement of the heavy and light chains, and somatic mutation combined with tight transcription and translational control of maturing antibodies results in a complicated process for B-cell maturation. Once the B-cell has matured into an antibody presenting cell, the proper assembly of the expressed antibody is critical to its activity. To address this issue, the secretory machinery of the cell plays a vital role in the proper folding and timing of folding of assembly. The two heavy chains are linked together by disulfide bonds such that in any naturally occurring antibody molecule, the two heavy chains and two light chains are identical. Proteolytic enzymes such as papain can be used to fragment the Ig molecule into three fragments. Two fragments are identical and contain the antigen binding activity and are referred to as Fab fragments, or Fragment antigen binding, corresponding to the paired light chain with a VH and CH1 domains. The third fragment contains no antigen binding and is referred to as the Fc or fragment crystallizable which contains the paired, disulfide linked CH2 and CH3 domains. The hinge region that links the Fab and Fc portions of the antibody is a flexible tether, allowing independent movement of the two Fab regions which would not be possible if the tether were rigid. Transport of this multimeric complex is dependent on the correct assembly of the component parts, which is controlled, in part, by the association of incompletely assembled Ig heavy chains with the endoplasmic reticulum (ER) chaperone, BiP. (Lee Y. K,. et. al., Mol Biol Cell. 1999 Jul;10(7):2209-19) Although other heavy chain-constant domains interact transiently with BiP, in the absence of light chain synthesis, BiP binds stably to the first constant domain (C_{H}1) of the heavy chain, causing it to be retained in the ER. In the absence of light chain expression, the C_{H}1 domain neither folds nor forms its intradomain disulfide bond and therefore remains a substrate for BiP. In vivo, light chains are required to facilitate both the folding of the C_{H}1 domain and the release of BiP. (Lee Y K, et. al., Mol Biol Cell. 1999 Jul;10(7):2209-19) Light chains are not intrinsically essential for C_{H}1 domain folding, but play acritical role in removing BiP from the C_{H}1 domain, thereby allowing it to fold and Ig assembly to proceed. The assembly of multimeric protein complexes in the ER is not strictly dependent on the proper folding of individual subunits; rather, assembly can drive the complete folding of protein subunits. It has been demonstrated that BiP and light chain cooperate to ensure that only properly assembled Ig molecules are transported from the ER by controlling the final folding of the heavy chain. Therefore the requirement for presence of both chains in the same cell, in the same sub-cellular organelle, at the same amount at the same time is critical for maximal throughput of mature antibody.

The standard recombinant expression of antibodies as a type of multimeric proteins has paralleled the approach provided by the mammalian antibody source, which follows the two genes for two polypeptides rule, as each chain of the antibody is expressed from an individual gene encoding each chain. The transcription, translation and cellular localization or secretion of each chain is controlled independently of its corresponding chain. As such, each polypeptide chain of the antibody multimer is controlled by separate promoters and secretory leaders. Differences in the chromosome insertion points, promoter strength and timing as well as the efficiency of secretory peptides can result in varying levels of each chain being present at a given time in the endoplasmic reticulum (ER), resulting in incomplete or delayed maturation of antibodies because the absence or decreased levels of the counterpart chain. Effects of insertion positions, whether proximal to endogenous promoters or enhancers, differential promoter efficiencies, translocation efficiencies and translational kinetics can result in aberrant accumulation of the recombinant antibody in foreign systems. The one gene, one polypeptide rule is occasionally broken for reasons of efficiency, as often is the case with viruses, and proper folding as dictated by the more complex proteins and for temporal control as for otherwise toxic or regulatory molecules such as prohormornes in the form of proproteins.

Recently, expression and assembly in transgenic plants of foreign multimeric proteins, such as antibodies, has been demonstrated by the work of Hein et al., U.S. Pat. No. 6,417,429 and USPA 20030172407. However, as depicted in FIG. 1, the process is complex and requires considerable time and experimentation. Specifically, as shown in FIG. 1, two separate genes are constructed, each gene encodes a portion of a desired antibody such that the first gene includes a promoter (Pr), a signal peptide (Sp) and a segment that expresses a heavy chain and the second gene includes a promoter, a signal peptide and a segment that expresses a light chain. The first gene is inserted into cells of a first plant, and the second gene is inserted into cells of a second plant. Thereafter, the first and second plant are cross pollinated in order to generate progeny that hopefully includes both the first and second genes and will therefore cause expression of a proprotein that will fold to form an antibody of interest.

One of many difficulties associated with the methodology set forth in Hein et al., U.S. Pat. No. 6,417,429 and USPA 20030172407, is that considerable time may be required to allow the first and second plants to grow, subsequently cross pollinate and generate progeny. Further, it is possible that the progeny may not include the desired combination of genes for expressing both the light and heavy chains.

The viral vector plant expression system of TMV utilizes endogenous and heterologous viral promoters to drive the expression of foreign genes. The vector easily accommodates a single foreign gene, but has more difficulties with additional genes as the size becomes an issue as well as the position effects of additional promoters required to produce an additional polypeptide as is required for antibodies. With the viral vector, the farther the promoter/gene set is from the 3′ end of the genome the lower the transcriptional activity. Therefore the larger the insert the lower the expression as a result of the intervening sequences of the heterologous gene. As for heterodimers as is the case for Fab's, the simultaneous expression of stoichiometric levels of heavy and light chains is essential for secretion. This is a result from the documented role of the chaperone BiP in the maturation of antibodies. BiP has a role in retaining the nascent chain in the oxidizing environment of the ER until the counterpart chain interacts, becomes disulfide linked and subsequently released from the ER resulting in the accumulation of the antibody in the secretory fluid. The heavy and light chains must be expressed at comparable levels as the resulting heterodimer contains a one to one ratio of heavy and light chains. Attempts have been made to express one chain from one vector and the second chain form a second vector (Verch T, et al., J Immunol Methods. 1998 Nov. 1;220(1-2):69-75). The two vectors were used to super-infect a plant and small amounts of antibodies were recovered. This approach is problematic because of cross-protection of an infected cell with one virus from being infected with a second virus. Typically, only the monolayer of cells present at the confluence of infections are thought to be simultaneously infected with both viruses. In additional an ER retention signal was placed on the chains to facilitate association by retained co-localization of the chains.

It is now generally accepted that proteins destined for secretion from eukaryotic cells are translocated to the endoplasmic reticulum due to the presence of a signal sequence which is cleaved off by the enzyme signal peptidase located in the rough ER membrane. The protein is then transported from the ER to the Golgi and via Golgi derived secretory vesicles to the cell surface (S. Pfeffer and J. Rothman, Ann. Rev. Biochem. 56:289-52, 1987). Another major step in the production of correctly processed and correctly folded proteins is the conversion of proproteins to the mature forms in the Golgi apparatus and secretory vesicles. The cleavage of the proprotein occurs at a so-called dibasic site, i.e. a motif consisting of at least two basic amino acids. The processing is catalyzed by enzymes located in the Golgi apparatus, the so-called “dibasic processing endoproteases”. There are different “dibasic processing endoproteases” known which are involved in the processing of precursor, for example the mammalian proteases furin, PC2, PC1 and PC3, (Barr, Cell 66:1-3, 1991) and the product of the yeast YAP3 gene (Egel-Mitani et al., Yeast 6:127-137 1990) and yeast yscF (also named KEX2 gene product; KEX2p). KEX2p is involved in the maturation of the yeast mating pheromone, alpha-mating factor (J. Kurjan and I. Hershkowitz, Cell 30:933-934, 1982). The alpha-mating factor is produced as a 165 amino acid precursor which is processed during the transport to the cell surface. In the first step, a 19-amino acid signal sequence (pre-sequence) is cleave off by the signal peptidase. Then the precursor is glycosylated and moves to the Golgi where a 66 amino acid pro-sequence is cutoff by KEX2p. The alpha-mating factor precursor is also known as alpha factor “leader” sequence. A second protease in the Golgi apparatus, i.e. the KEX1 gene product is responsible for the final maturation of the protein.

BiP, like all hsp70 family members, binds to unfolded nascent polypeptides and is thought to function by recognizing hydrophobic sequences exposed on unfolded or unassembled polypeptides and, by inhibiting intra- or intermolecular aggregation, maintaining them in a state competent for subsequent folding and oligomerization.(Knarr G, et. Al., J Biol Chem. 1995 Nov. 17;270(46):27589-94) BiP recognizes heptapeptides and prefers those with aliphatic residues (Flynn G C, et al., Nature. 1991 Oct. 24;353(6346):726-30) where the aliphatic residues were preferred only for alternating residues, suggesting that if a protein containing this sequence was extended, the hydrophobic residues would all face the same direction and perhaps fit in to the BiP polypeptide-binding pocket.

Plant seed toxins such as Ricin from castor beans utilize a preproprotein expression strategy to mitigate the toxic effects of ricin by having an inactive proprotein. The proricin is moved through the ER and Golgi complex to the protein storage vacuoles (PSV) of the bean. Once in the PSV, resident proteases mature the protein to produce a highly toxic heterodimer composed of A and B chains linked by a disulfide bond. (Vitale, A and Denecke, J. Plant Cell. 1999 Apr.;1 1(4):615-28) A similar strategy can be envisioned as a useful strategy for the expression of recombinant multimeric proteins that in their mature form would be toxic or otherwise detrimental to the host. An antibody that recognized an essential receptor may be such a molecule. The expression of the multimeric or heterodimeric protein as an inactive proprotein precursor and delivery of immature proprotein to a organelle such as the PSV followed by the subsequent removal of the propeptide to activate the antibody or other molecule would reduce or eliminate the toxic effects of that molecule.

To address the more complex folding requirements of certain heterodimers, nature has devised a strategy of incorporating folding intermediates that act as additional folding chaperone domains referred to as propeptides. Pro-sequence can be any sequence which can act as a molecular chaperone, i.e. a polypeptide which in cis or trans can influence the formation of an appropriate conformation, but is by in large not present in the mature form of the protein. These proproteins are folded as immature protein intermediates, facilitating proper conformation and disulfide linkages in the ER. Once the folding of the stable intermediate has been accomplished by concert of the endogenous chaperone proteins in conjunction with the propeptide domain as part of the proprotein whole, the propeptide is removed in the Golgi from the proprotein to generate a mature active protein. This is the case for many proteins such as insulin, Saccharomyces cerevisiae killer toxin virus (ScV) k1 toxin, Kluyveromyces lactis plasmid k1 toxin, and the KP6 toxin of Ustilago maydis virus(UmV). The insulin C chain is removed to produce the mature, active hormone in newly formed clathrin coated secretory vessicles. The Saccharomyces cerevisiae K1 killer toxin precursor is composed of a signal peptide, alpha subunit, a propeptide (gamma subunit), and a beta subunit. The secreted precursor protein is folded with inter- and intra-chain disulfide bonds formed with the alpha and beta subunits, and the gamma propeptide is removed by proteolysis. The mature K1 toxin is a heterodimeric protein composed of disulfide linked alpha and beta polypeptides. Similarly, the KP6 toxin consists of two distinct polypeptides, alpha and beta, but differ in that the subunits are not covalently associated, encoded by a 657 base pair double stranded RNA segment. A single transcript produces a 219 amino acid KP6 preprotoxin, which is then processed to produce the 78 amino acid alpha and the 81 amino acid beta polypeptides. In virtually infected U. maydis cells, processing of the protoxin by Kex2p occurs after the Pro-Arg residues at position 27 and the Lys-Arg residues at 107 to generate alpha and at 139 to generate beta.

The expression of a multimeric protein in plant cells requires that the genes coding for the polypeptide chains be present in the same plant cell. Until the advent of the procedures disclosed herein, the probability of actually introducing both genes into the same cell was extremely remote. Assembly of multimeric protein and expression of significant amounts of same has now been made feasible by use of the methods and constructs described herein.

In accordance with the present invention described hereinbelow, it is possible to avoid some of the difficulties associated with the methods disclosed in Hein et al., U.S. Pat. No. 6,417,429 and USPA 20030172407 and produce a desired antibody using a single gene, not two separate genes.

### SUMMARY OF THE INVENTION

Therefore, methods of producing active biomolecules with relative ease and in large quantities are now disclosed. In addition, the molecules and compositions produced thereby are disclosed as well.

To solve these problems, a class of novel, artificial preproproteins has now been designed and engineered which comprise a proprotein, that is, a protein assembly capable of producing a multimeric protein from a single protein comprised of a first peptide, a second peptide and propeptide, where the first peptide and the second peptide associate to assume a biologically functional conformation essentially free of the propeptide. Examples of the first peptide and second peptide would be the light and heavy chain of an immunoglobulin molecule, the light chain and a fragment of the heavy chain immunoglobulin molecule, the alpha and beta chain of the T cell receptor, or the alpha and beta chains of hemoglobin. Examples of the propeptide would be the insulin C chain, Saccharomyces cerevisiae K1 killer toxin propeptide (gamma subunit), Kluyveromyces lactis plasmid k1 toxin propeptide or the KP6 toxin propeptide chain. This invention features artificial, proproteins which fold to form a stable intermediate protein containing a propeptide, where the mature multimeric protein has subunits with an associative properties, DNA encoding these proteins prepared by recombinant techniques, host cells harboring these DNAs, and methods for the production of these proteins and DNAs.

The conversion of a multimeric protein from the naturally occurring two genes for two polypeptides to a proprotein where one gene results in two polypeptides. The creation of a proprotein that results in the accumulation of a properly folded, properly associated multimeric protein would be advantageous. This artificial proprotein must drive the formation of stable folding intermediates such that appropriate intra- and inter-chain interactions or associations such as covalent and non-covalent linkages are formed. The pre-peptide or signal peptide directs the nascent polypeptide to the ER through interaction with the signal recognition particle and the signal peptide is subsequently cleaved in the ER by the signal peptidase. While resident in the ER, the complex secondary, tertiary and quaternary folding must take place as the molecular chaperones, such as heat shock protein 70 (HSP70) family, which includes the binding protein (BiP), protein disulfide isomerase (PDI), which catalyses the formation of disulfide bridges, calnexin, calreticulin and glucosyl transferase, which specifically interact with nascent glycoproteins, are resident only in the rough ER. Once the stable, properly folded and disulfide linked proprotein is facilitated by the propeptide, it is transported to the Golgi apparatus for further processing. In the Golgi, the propeptide is proteolytically removed rendering the mature antibody in its active form, at which time it is transported out of the cell where it accumulates in the extracellular space or apoplast in plants. Proteolytic cleavage at the amino and carboxy termini of the propeptide by proteases results in the release of the propeptide. The Kex2 like protease recognition sequence has amino acid residues of lysine at P2 and arginine at P1, using the nomenclature convention of Schechter, I and Berger, A Biochem. Biophys. Res. Com. (1967) 27:157-62. The cleavage of the propeptide results in a carboxy terminal Lys-Arg amino acid pair remaining on the first peptide of interest. Proline or arginine can also be substituted for Lysine at the P2 position to make a Pro-Arg or Arg-Arg pair. The non-native pair may be created by addition of a single amino acid to make the cleavage site. A multimeric protein made by the method of the present invention will be characterized by its carboxy terminal lys-Arg, Pro-Arg or Arg-Arg on the first peptide. There are many different proteases that occur in different organisms. These proteases have varying specificities. Any amino acid pair that results from proteolytic cleavage of the propeptide is contemplated by this invention. The Lys-Arg, Pro-Arg or Arg-Arg pair may be retained or removed. A single Arg at the P1 position may also be removed without removing the amino acid at the P2 position. The derivative proteins made by removal of the amino acid pair are also contemplated by this invention. The propeptide facilitates the intersubunit interactions of the multimeric protein, whether the interactions are covalent, as in an antibody or non-covalent, electrostatic forces, hydrogen bonds, or Van der Waals forces and hydrophobic forces as in hemoglobin. Once the associative interaction has occurred the propeptide is then removed to release the desired multimeric protein.

This patent describes the creation of a chimeric proprotein where the polypeptide subunits of the UmV KP6 toxin are removed and replaced by polypeptides subunits from a multimeric protein not naturally found as a proprotein, such as immunoglobulin, containing the immunoglobulin light and heavy chains, which directs the synthesis of an artificial proprotein where the proprotein folds to form a stable intermediate and the propeptide is subsequently removed from the proprotein rendering a mature, active multimeric protein essentially free of the propeptide.

In a first embodiment of the invention an artificial proprotein includes three peptide sequences, a first peptide, an intermediate propeptide and a second peptide. This invention does not include peptides that are naturally bound to a propeptide, such as the insulin molecule. The present invention allows us to make proprotein configurations that are not found in nature. These configurations simplify the production of multimeric proteins by allowing them to be placed in a single gene configuration.

In another embodiment of the invention, an artificial polynucleotide includes four nucleotide sequences. The three-peptide configuration described above is attached to a preceding signal peptide.

In another embodiment of the invention a method of making an artificial polynucleotide, includes providing first, second, and third nucleotide sequences each encoding a first peptide of interest, an internal propeptide and a second peptide of interest, respectively. The nucleotide sequence that encodes a first peptide of interest can be the same as or different from the nucleotide sequence that encodes a second peptide of interest.

In another embodiment of the invention a method of making an artificial polynucleotide, includes providing a first, a second, a third and a fourth nucleotide sequence that encode a signal peptide sequence, a first peptide of interest, a propeptide and a second peptide of interest, respectively. The nucleotide sequence that encodes a first peptide of interest can be the same as or different from the nucleotide sequence that encodes a second peptide of interest.

In another embodiment of the invention a method of making an artificial proprotein, includes making an artificial polynucleotide that encodes the proprotein; and expressing the artificial polynucleotide in a host organism whereby the proprotein is made.

In another embodiment of the invention a method of making an artificial preproprotein, includes making an artificial polynucleotide that encodes the preproprotein; and expressing the artificial polynucleotide in a host organism.

In a another embodiment of the invention a method of making and isolating a multimeric protein, includes the steps of:

providing a first, a second, a third and a fourth nucleotide sequence that encode a signal peptide sequence, a first peptide of interest, a propeptide and a second peptide of interest, respectively;

connecting the 3′ terminus of the first nucleotide sequence to the 5′ terminus of the second nucleotide sequence;

connecting the 3′ terminus of the second nucleotide sequence to the 5′ terminus of the third nucleotide sequence; and

connecting the 3′ terminus of the third nucleotide sequence to the 5′ terminus of the fourth nucleotide sequence, so that an artificial polynucleotide results and is comprised of the four nucleotide sequences, and wherein the nucleotide sequence that encodes a first peptide of interest can be the same as or different from the nucleotide sequence that encodes a second peptide of interest;

introducing the resulting artificial polynucleotide into a host organism by transfection, or by stable transformation;

allowing the artificial polynucleotide to be expressed in the host organism whereby a preproprotein is made;

allowing the preproprotein to be processed into a mature multimeric protein, and isolating the multimeric protein.

The multimeric protein can be any multimeric protein having at least two peptide sequences that are intended to form a multimer but are usually encoded on different gene sequences, or do not naturally have a propeptide sequence between them. The peptides can be any set of peptides that are designed by the engineer to form a multimer. The host organism can be any host organism. Common host organisms are animal cells, human cells, animal tissues or whole animals, plant cells, plant tissues and whole plants.

In a first embodiment of the invention a vector encoding an artificial preproprotein, includes a nucleotide sequence necessary for replication of the vector nucleotides and proteins and an artificial polynucleotide inserted into the vector, that comprises a first nucleotide sequence that encodes a signal peptide sequence; a second nucleotide sequence that encodes a first peptide of interest, second nucleotide sequence being connected to the 3′ terminus of the first nucleotide sequence; a third nucleotide sequence that encodes a propeptide, third nucleotide sequence being connected to the 3′ terminus of the second nucleotide sequence; and a fourth nucleotide sequence that encodes a second peptide of interest, fourth nucleotide sequence being connected to the 3′ terminus of the third nucleotide sequence, the artificial polynucleotide inserted into the vector so that the vector can reproduce and, if required, can produce the artificial preproprotein.:

In another embodiment of the invention a transiently transformed cell, includes a vector encoding an artificial preproprotein. The nucleotide sequence necessary for replication of the vector nucleotides and proteins, an artificial polynucleotide encoding an artificial preproprotein inserted into the vector, the artificial polynucleotide comprising, a first nucleotide sequence that encodes a signal peptide sequence, a second nucleotide sequence that encodes a first peptide of interest, second nucleotide sequence being connected to the 3′ terminus of the first nucleotide sequence, a third nucleotide sequence that encodes a propeptide, third nucleotide sequence being connected to the 3′ terminus of the second nucleotide sequence; and a fourth nucleotide sequence that encodes a second peptide of interest, fourth nucleotide sequence being connected to the 3′ terminus of the third nucleotide sequence, the artificial polynucleotide inserted into the vector so that the vector can reproduce and, if required can produce the artificial preproprotein a promoter capable of directing expression of the artificial preproprotein, and the artificial preproprotein encoded by the artificial polynucleotide. Several different kinds of multimeric proteins are described below.

In a another embodiment of the invention a transgenic cell, includes:

(a) an artificial polynucleotide stably incorporated onto a chromosome, the artificial polynucleotide comprising:

a first nucleotide sequence that encodes a signal peptide sequence;

a second nucleotide sequence that encodes a first peptide of interest, second nucleotide sequence being connected to the 3′ terminus of the first nucleotide sequence;

a third nucleotide sequence that encodes a propeptide, third nucleotide sequence being connected to the 3′ terminus of the second nucleotide sequence; and

a fourth nucleotide sequence that encodes a second peptide of interest, fourth nucleotide sequence being connected to the 3′ terminus of the third nucleotide sequence, the artificial polynucleotide inserted into the vector so that the vector can reproduce and, if required can produce the artificial preproprotein.

(b) a promoter capable of directing expression of the artificial preproprotein; and

(c) the artificial preproprotein encoded by the artificial polynucleotide.

In a another embodiment of the invention a transgenic plant, includes plant cells containing an artificial polynucleotide sequence encoding an artificial preproprotein that artificial preproprotein comprises a) a signal peptide sequence, b) an immunoglobulin heavy chain or light chain peptide, c) a propeptide, and d) an immunoglobulin heavy chain or light chain peptide, wherein the heavy chain can be in either the b or the d position on the preproprotein, and the light chain will be on the other position, wherein the artificial preproprotein contains a signal peptide sequence signal peptide sequence forming a secretion signal containing immunoglobulin molecules encoded by said artificial polynucleotide sequence, wherein said signal peptide sequence signal peptide sequence is cleaved from said artificial preproprotein by proteolytic processing, and wherein said propeptide is cleaved from the heavy chain and the light chain following proper folding of the remaining polypeptide. The immunoglobulin example is one of many possible examples of a multimeric protein that can be made by a transgenic plant. Any other set of peptides necessary to make a multimeric protein would also be suitable.

In an another embodiment of the invention a method for making a transgenic plant capable of producing immunoglobulin molecules, includes:

(a) introducing into the genome of a member of a plant species an artificial polynucleotide sequence encoding a preproprotein that preproprotein comprises (i) a signal peptide sequence, (ii) an immunoglobulin heavy chain or light chain peptide, (iii) a propeptide, and d) an immunoglobulin heavy chain or light chain peptide, wherein the heavy chain can be in either the b or the d position on the preproprotein, and the light chain will be on the other position; and

(b) allowing stable transformation to occur to produce a transformant. The immunoglobulin example is one of many possible examples of a multimeric protein that can be made by a transgenic plant. Any other set of peptides necessary to make a multimeric protein would also be suitable.

A process for producing an immunoglobulin molecule or an immunologically functional immunoglobulin fragment comprising at least the variable domains of the immunoglobulin heavy and light chains, in a single host cell, comprising the steps of:

(a) transforming said single host cell with a single DNA sequence encoding at least the variable domain of the immunoglobulin heavy chain, a propeptide and at least the variable domain of the immunoglobulin light chain, and

(b) expressing said single DNA sequence so that said immunoglobulin heavy and light chains are produced as a single propeptide molecule in said transformed single host cell.

In another embodiment of the invention a vector includes a single DNA sequence encoding at least a variable domain of an immunoglobulin heavy chain and at least a variable domain of an immunoglobulin light chain wherein said single DNA sequence is located in said vector at a single insertion site.

In a another embodiment of the invention a transformed host cell includes at least two vectors, at least one of said vectors comprising a single DNA sequence encoding at least a variable domain of an immunoglobulin heavy chain and at least the variable domain of an immunoglobulin light chain.

In a another embodiment of the invention a method includes:

(a) preparing a DNA sequence consisting essentially of DNA encoding an immunoglobulin consisting of an immunoglobulin heavy chain and light chain or Fab region, said immunoglobulin having specificity for a particular known antigen, wherein the DNA sequence incorporates an artificial polynucleotide encoding a proprotein which consists of at least a variable domain of an immunoglobulin heavy chain, a cleavable propeptide, and at least the variable domain of an immunoglobulin light chain;

(b) inserting the DNA sequence of step a) into a replicable expression vector operably linked to a suitable promoter;

(c) transforming a prokaryotic or eukaryotic microbial host cell culture with the vector of step b);

(d) culturing the host cell; and

(e) recovering the immunoglobulin from the host cell culture, said immunoglobulin being capable of binding to a known antigen.

In a another embodiment of the invention a process for producing an immunoglobulin molecule or an immunologically functional immunoglobulin fragment includes at least the variable domains of the immunoglobulin heavy and light chains, in a single host cell, comprising:

expressing a single DNA sequence encoding at least the variable domain of the immunoglobulin heavy chain and at least the variable domain of the immunoglobulin light chain so that said immunoglobulin heavy and light chains are produced as a single proprotein molecule in said single host cell transformed with said single DNA sequence.

In another embodiment, a multimeric protein is characterized by a first and second peptides, the first peptide comprising a non-native amino acid pair at the P1 and P2 positions of the carboxy terminus.

A multimeric protein derived from a multimeric protein is characterized by a first and second peptides, the first peptide comprising a non-native amino acid pair at the P1 and P2 positions of the carboxy terminus.

General References

Unless otherwise indicated, the practice of many aspects of the present invention employs conventional techniques of molecular biology, recombinant DNA technology and immunology, which are within the skill of the art. Such techniques are described in more detail in the scientific literature, for example, Sambrook, J. et al., Molecular Cloning: A Laboratory Manual, 2^{nd} Ed., Cold Spring Harbor Press, Cold Spring Harbor, N.Y., 1989; Ausubel, F. M. et al. Current Protocols in Molecular Biology, Wiley-Interscience, New York, current volume; Albers, B. et al., Molecular Biology of the Cell, 2^{nd} Ed., Garland Publishing, Inc., New York, N.Y. (1989); Lewin, B M, Genes IV, Oxford University Press, Oxford (1990); Watson, J. D. et al., Recombinant DNA, Second Edition, Scientific American Books, New York, 1992; Darnell, JOE et al., Molecular Cell Biology, Scientific American Books, Inc., New York, N.Y. (1986); Old, R. W. et al., Principles of Gene Manipulation: An Introduction to Genetic Engineering, 2^{nd} Ed., University of California Press, Berkeley, Calif. (1981); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); Methods in Enzymology: Guide to Molecular Cloning Techniques (Berger and Kimball, eds., 1987); Hartlow, E. et al., Antibodies: A Laboratory Manual, Cold Spring Harbor Laboratory Press, Cold Spring Harbor, N.Y., 1988), Collegian, J. E. et al., eds., Current Protocols in Immunology, Wiley-Interscience, New York 1991. Protein structure and function is discussed in Schulz, G E et al., Principles, of Protein Structure, Springer-Verlag, New York, 1978, and Creighton, T E, Proteins: Structure and Molecular Properties, W.H. Freeman & Co., San Francisco, 1983.

### BRIEF DESCRIPTION OF THE DRAWINGS

FIG. 1 is a block diagram showing prior art methods for expressing antibodies in plants where two genes are employed initially in two separate plants, the two plants subsequently being cross pollinated to produce progeny that may produce a desired protein in the endoplasmic reticulum.

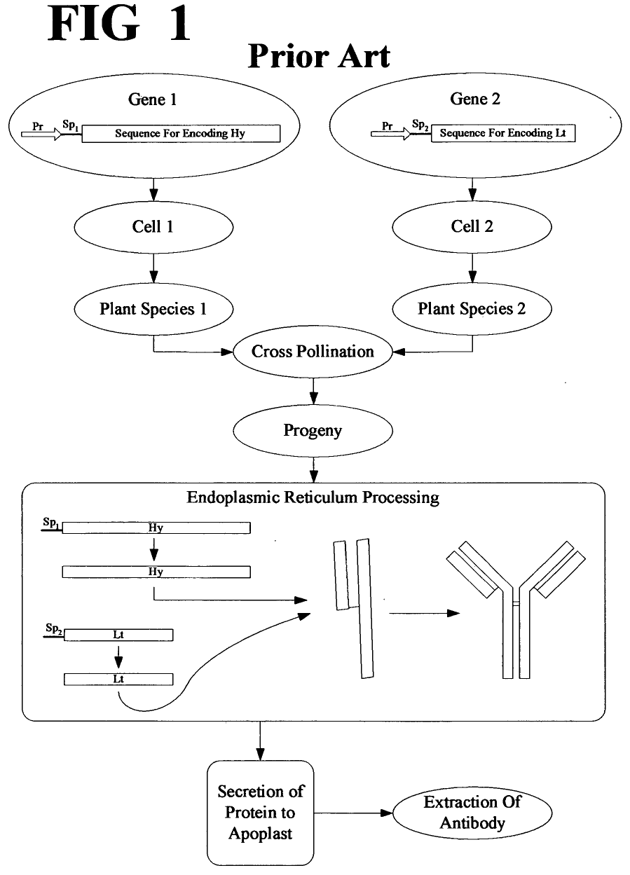

FIG. 2 is a flowchart generically showing the basic steps for producing a construct that includes in the following order a promoter sequence, signal peptide, a light chain sequence, a propeptide and a heavy chain sequence, in accordance with the present invention.

FIG. 3 is a flowchart generically showing the basic steps for producing a construct that includes in the following order a promoter sequence, signal peptide, a heavy chain sequence, a propeptide and a light chain sequence, in accordance with the present invention.

FIG. 4 is a block diagram representing a one embodiment of the present invention where a construct similar to the construct depicted in FIG. 3, for encoding a preproprotein is introduced into cells. In this embodiment, the construct includes a short heavy chain is inserted between a signal peptide (Sp) and a propeptide. After expression, the signal peptide (Sp) is removed within the endoplasmic reticulum to produce the preproprotein. Subsequent maturation within the Golgi of the cell removes the propeptide thereby producing a folded desired antibody fragment or Fab.

FIG. 5 is a block diagram representing another embodiment of the present invention where a construct encoding a preproprotein is introduced into cells. In this embodiment, the construct is similar to the construct depicted in FIG. 2 and includes a light chain inserted between the signal peptide (Sp) and the propeptide. After expression, the signal peptide (Sp) is removed within the endoplasmic reticulum to produce the preproprotein. Subsequent maturation within the Golgi of the cell removes the propeptide thereby producing a folded desired antibody fragment or Fab.

FIG. 6 is a block diagram representing yet another embodiment of the present invention where a single construct encoding a preproprotein is introduced into cells. In this embodiment, the sequence for encoding a longer heavy chain is inserted between the signal peptide (Sp) and the propeptide. After expression, the signal peptide (Sp) is removed within the endoplasmic reticulum to produce the preproprotein. Subsequent maturation within the Golgi of the cell removes the propeptide thereby producing a folded desired Fab′.

FIG. 7 is a block diagram representing a further embodiment of the present invention where a single construct encoding a preproprotein is introduced into cells where the construct includes a light chain is inserted between a signal peptide (Sp) and a propeptide with a longer heavy chain attached to the other end of the propeptide. After expression, the signal peptide (Sp) is removed within the endoplasmic reticulum to produce the preproprotein. Subsequent maturation within the Golgi of the cell removes the propeptide thereby producing a folded desired Fab′.

FIG. 8 is a block diagram representing a still another embodiment of the present invention where a single construct encoding a preproprotein is introduced into cells where the construct includes a full heavy chain is inserted between the signal peptide (Sp) and the propeptide. After expression, the signal peptide (Sp) is removed within the endoplasmic reticulum to produce the preproprotein. Subsequent maturation within the Golgi of the cell removes the propeptide thereby producing a folded desired antibody.

FIG. 9 is a block diagram representing a yet still another embodiment of the present invention where a single construct encoding a preproprotein is introduced into cells. In this embodiment, the construct includes a light chain between the signal peptide (Sp) and the propeptide with a full heavy chain attached to the other end of the propeptide. After expression, the signal peptide (Sp) is removed within the endoplasmic reticulum to produce the preproprotein. Subsequent maturation within the Golgi of the cell removes the propeptide thereby producing a folded desired antibody.

FIG. 10 is a block diagram showing various platforms that may be utilized for the production of a polypeptide using a single construct encoding preproprotein construct in accordance with the present invention, where the preproprotein includes a signal peptide (Sp), a light chain attached to the signal peptide, a proprotein attached to the light chain and a heavy chain attached to the proprotein.

FIG. 11 is a block diagram similar to FIG. 10, showing various platforms that may be utilized for the production of a polypeptide using a single gene encoding preproprotein construct in accordance with the present invention, where the preproprotein includes a signal peptide (Sp), a heavy chain attached to the signal peptide, a proprotein attached to the heavy chain and a light chain attached to the proprotein.

### DETAILED DESCRIPTION OF THE INVENTION

Definitions

Dicotyledon (dicot): A flowering plant whose embryos have two seed halves or cotyledons. Examples of dicots are: tobacco; tomato; the legumes including alfalfa; oaks; maples; roses; mints; squashes; daisies; walnuts; cacti; violets; and buttercups.

Monocotyledon (monocot): A flowering plant whose embryos have one cotyledon or seed leaf. Examples of monocots are: lilies; grasses; corn; grains, including oats, wheat and barley; orchids; irises; onions and palms.

Lower plant: Any non-flowering plant including ferns, gymnosperms, conifers, horsetails, club mosses, liver warts, hornworts, mosses, red algae, brown algae, gametophytes, sporophytes of pteridophytes, and green algae.

Eukaryotic hybrid vector: A DNA by means of which a DNA coding for a polypeptide (insert) can be introduced into a eukaryotic cell.

Extrachromosomal ribosomal DNA (rDNA): A DNA found in unicellular eukaryotes outside the chromosomes, carrying one or more genes coding for ribosomal RNA and replicating autonomously (independent of the replication of the chromosomes).

Palindromic DNA: A DNA sequence with one or more centers of symmetry.

T-DNA: A segment of transferred DNA.

rDNA: Ribosomal DNA.

rRNA: Ribosomal RNA.

Ti-plasmid: Tumor-inducing plasmid.

Ti-DNA: A segment of DNA from Ti-plasmid.

Insert: A DNA sequence foreign to the DNA clone it is being inserted into.

Structural gene: A gene coding for a polypeptide and being equipped with a suitable promoter, termination sequence and optionally other regulatory DNA sequences, and having a correct reading frame.

Signal sequence: A DNA sequence coding for a signal peptide attached to the polypeptide.

Signal peptide: A series of amino acids attached to the polypeptide which binds the polypeptide to the endoplasmic reticulum and is essential for protein secretion. This signal may also be referred to herein as a prepeptide. The term “signal peptide” may also be used to refer to the sequence of amino acids that determines whether a protein will be formed on the rough endoplasmic reticulum or on free ribosomes.

(Selective) Genetic marker: A DNA sequence coding for a phenotypic trait by means of which transformed cells can be selected from untransformed cells.

Promoter: A recognition site on a DNA or RNA sequence or group of DNA or RNA sequences that provide an expression control element for a gene and to which RNA polymerase specifically binds and initiates RNA synthesis (transcription) of that gene.

Inducible promoter: A promoter where the rate of RNA polymerase binding and initiation is modulated by external stimuli. Such stimuli include light, heat, anaerobic stress, alteration in nutrient conditions, presence or absence of a metabolite, presence of a ligand, microbial attack, wounding and the like.

Viral promoter: A promoter with a DNA or RNA sequence substantially similar to the promoter found at the 5′ end of a viral gene. A typical viral promoter is found at the 5′ end of the gene coding for the p21 protein of MMTV described by Huang et al., Cell 27: 245 (1981).

Synthetic promoter: A promoter that was chemically synthesized rather than biologically derived. Usually artificial promoters incorporate sequence changes that optimize the efficiency of RNA polymerase initiation.

Constitutive promoter: A promoter where the rate of RNA polymerase binding and initiation is approximately constant and relatively independent of external stimuli. Examples of constitutive promoters include the cauliflower mosaic virus 35S and 19S promoters described by Poszkowski et al., EMBO J. 3: 2719 (1989) and Odell et al., Nature 313: 810 (1985).

Temporally regulated promoter: A promoter where the rate of RNA polymerase binding and initiation is modulated at a specific time during development. Examples of temporally regulated promoters are given in Chua et al., Science 244: 174-181 (1989).

Spatially regulated promoter: A promoter where the rate of RNA polymerase binding and initiation is modulated in a specific structure of the organism such as the leaf, stem or root. Examples of spatially regulated promoters are given in Chua et al., Science 244: 174-181 (1989).

Spatiotemporally regulated promoter: A promoter where the rate of RNA polymerase binding and initiation is modulated in a specific structure of the organism at a specific time during development. A typical spatiotemporally regulated promoter is the EPSP synthase-35S promoter described by Chua et al., Science 244: 174-181 (1989).

Chelating agent: A chemical compound, peptide or protein capable of binding a metal. Examples of chelating agents include ethylene diamine tetra acetic acid (EDTA), ethyleneglycol-bis-(beta-aminoethyl ether) N,N,N′,N′-tetraacetic acid (EGTA), 2,3-dimercaptopropanel-1-sulfonic acid (DMPS), and 2,3-dimercaptosuccinic acid (DMSA), and the like.

Metal chelation complex: A complex containing a metal bound to a chelating agent.

Immunoglobulin product: A polypeptide, protein or multimeric protein containing at least the immunologically active portion of an immunoglobulin heavy chain and is thus capable of specifically combining with an antigen. Exemplary immunoglobulin products are an immunoglobulin heavy chain, immunoglobulin molecules, substantially intact immunoglobulin molecules, any portion of an immunoglobulin that contains the paratope, including those portions known in the art as Fab fragments, Fab′ fragment, F(ab′)₂ fragment and Fv fragment.

Immunoglobulin molecule: A multimeric protein containing the immunologically active portions of an immunoglobulin heavy chain and immunoglobulin light chain associated with each other and capable of specifically combining with antigen.

Fab fragment (Fab): A multimeric protein consisting of the portion of an immunoglobulin molecule containing the immunologically active portions of an immunoglobulin heavy chain called the Fd and an immunoglobulin light chain associated with each other and capable of specifically combining with antigen. Fab fragments are typically prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using methods well known in the art.

Fab′ fragment (Fab′): An Fab that dimerizes or a dimeric Fab.

Asexual propagation: Producing progeny by regenerating an entire plant from leaf cuttings, stem cuttings, root cuttings, single plant cells (protoplasts) and callus.

Glycosylated core portion: The pentasaccharide core common to all asparagine-linked oligosaccharides. The pentasaccharide care has the structure Manα-1-3(manα-1-6) Manβ-1-46LcNAcβ-1-4 6LcNac-(ASN amino acid). The pentasaccharide core typically has 2 outer branches linked to the pentasaccharide core.

N-acetylglucosamine containing outer branches: The additional oligosaccharides that are linked to the pentasaccharide core (glycosylated core portion) of asparagine-linked oligosaccharides. The outer branches found on both mammalian and plant glycopolypeptides contain N-acetylglucosamine in direct contrast with yeast outer branches that only contain mannose. Mammalian outer branches have sialic acid residues linked directly to the terminal portion of the outer branch.

Glycopolypeptide multimer: A globular protein containing a glycosylated polypeptide or protein chain and at least one other polypeptide or protein chain associated with each other to form a single globular protein. Both heterodimeric and homodimeric glycoproteins are multimeric proteins. Glycosylated polypeptides and proteins are n-glycans in which the C(1) of N-acetylglucosamine is linked to the amide group of asparagine.

Immunoglobulin superfamily molecule: A molecule that has a domain size and amino acid residue sequence that is significantly similar to immunoglobulin or immunoglobulin related domains. The significance of similarity is determined statistically using a computer program such as the Align program described by Dayhoff et al., Meth Enzymol. 524-545 (1983). A typical Align score of less than 3 indicates that the molecule being tested is a member of the immunoglobulin gene superfamily.

The immunoglobulin gene superfamily contains several major classes of molecules including those shown in Table A and described by Williams and Barclay, in Immunoglobulin Genes, p361, Academic Press, New York, N.Y. (1989).

TABLE A
The Known Members of The Immunoglobulin Gene Superfamily*

Immunoglobulin
Heavy chains
Light chain kappa
Light chain lambda
T cell receptor (Tcr) complex
Tcr α-chain
Tcr β-chain
Tcr gamma chain
Tcr X-chain
CD3 gamma chain
CD3 δ-chain
CD3ε-chain
Major histocompatibility complex (MHC) antigens
Class I H-chain
β₂-microglobulin
Class II α
Class II β
β₂-m associated antigens
TL H chain
Qa-2 H chain
CD1a H chain
T lymphocyte antigens
CD2
CD4
CD7
CD8 chain I
CD8 Chain IId
CD28
CTLA4
Haemopoietic/endothelium antigens
LFA-3
MRC OX-45
Brain/lymphoid antigens
Thy-1
MRC OX-2
Immunoglobulin receptors
Poly Ig R
Fc gamma 2b/gamma 1R
Fc.epsilon.RI(α-)
Neural molecules
Neural adhesion molecule (MCAM)
Myelin associated gp (MAG)
P₀ myelin protein
Tumor antigen
Carcinoembryonic antigen (CEA)
Growth factor receptors
Platelet-derived growth factor (PDGF) receptor
Colony stimulating factor-1 (CSF1) receptor
Non-cell surface molecules
α₁ B-glycoprotein
Basement membrane link protein
*See Williams and Barclay, in Immunoglobulin Genes, p361, Academic Press, NY (1989); and Sequences of Proteins of Immunological Interest, 4th ed., U.S. Dept. of Health and Human Serving (1987).

Catalytic site: The portion of a molecule that is capable of binding a reactant and improving the rate of a reaction. Catalytic sites may be present on polypeptides or proteins, enzymes, organics, organo-metal compounds, metals and the like. A catalytic site may be made up of separate portions present on one or more polypeptide chains or compounds. These separate catalytic portions associate together to form a larger portion of a catalytic site. A catalytic site may be formed by a polypeptide or protein that is bonded to a metal.

Enzymatic site: The portion of a protein molecule that contains a catalytic site. Most enzymatic sites exhibit a very high selective substrate specificity. An enzymatic site may be comprised of two or more enzymatic site portions present on different segments of the same polypeptide chain. These enzymatic site portions are associated together to form a greater portion of an enzymatic site. A portion of an enzymatic site may also be a metal.

Self-pollination: The transfer of pollen from male flower parts to female flower parts on the same plant. This process typically produces seed.

Cross-pollination: The transfer of pollen from the male flower parts of one plant to the female flower parts of another plant. This process typically produces seed from which viable progeny can be grown.

Epitope: A portion of a molecule that is specifically recognized by an immunoglobulin product. It is also referred to as the determinant or antigenic determinant.

Abzyme: An immunoglobulin molecule capable of acting as an enzyme or a catalyst.

Enzyme: A protein, polypeptide, peptide RNA molecule, or multimeric protein capable of accelerating or producing by catalytic action some change in a substrate for which it is often specific.

Light Chain (Lt): The smaller of two (MWt ca. 23000) of the two types of polypeptide chain in an immunoglobulin monomer and consists of one V and one C domain. There are two classes of light chain known as kappa and lambda.

Variable (V): Domain of the immunoglobulin monomer which contains relatively invariant framework regions and hypervariable regions. The framework regions provide a protein scaffold for the hypervariable regions that make contact with antigen.

Constant (C): Domain of the immunoglobulin monomer which is relatively constant in amino acid sequence between different immunoglobulin molecules and determines the particular effector function and the type such as alpha, gamma, delta, epsilon and mu corresponding to the classes IgA, IgG, IgD, IgE and IgM, respectively

Short Heavy Chain (Fd): The portion of the heavy chain molecule containing the immunologically active portion of the immunoglobulin heavy chain and consists of one V and one C domain.

Longer Heavy Chain (Fd′): The Fd portion of the heavy chain molecule containing the immunologically active portion of the immunoglobulin heavy chain and a dimerization domain. One type of dimerization domain is a C domain.

Heavy Chain (Hy): A class-specific polypeptide immunoglobulin component (MWt ca. 50000-70000, depending on Ig class). The various types of heavy chain are designated alpha, gamma, delta, epsilon and mu corresponding to the classes IgA, IgG, IgD, IgE and IgM, respectively.

Artificial: For purposes of this invention, artificial means an artificial arrangement of peptide or nucleotide domains, one of the domains being a propeptide or propeptide coding sequence, the arrangement having no known analog in nature. The arrangement is not found in nature, because the two domains bonded to the propeptide or propeptide coding sequence are not naturally arranged on a single open reading frame or a single resulting proprotein.

An artificial nucleotide sequence that encodes a proprotein is an arrangement of nucleotide sequence domains in an open reading frame, wherein one of the domains encodes an internal propeptide, the arrangement having no known analog in nature. An artificial proprotein sequence is an arrangement of peptide sequence domains in a proprotein wherein one of the domains is an internal propeptide, the arrangement having no known analog in nature. An example of an artificial nucleotide sequence that encodes a proprotein is an arrangement of nucleotide sequence domains in a single open reading frame, wherein one of the domains encodes an internal propeptide and the other two domains encode the heavy and light chains respectively of an antibody or Fab fragment. In nature two separate genes encode the heavy and light chains respectively of the antibody.

The artificial antibody proprotein sequence is an arrangement of peptide sequence domains. One of the domains is an internal propeptide. Flanking the internal propeptide are the light chain on one side of the propeptide and the heavy chain on the other side. This arrangement has no known analog in nature. The arrangement will result in a disulfide bonded multimeric protein upon folding and cleavage of the internal propeptide. By contrast, insulin is not an example of an artificial proprotein according to this invention. Insulin is a multimer that, in nature, is encoded on a single open reading frame. That open reading frame has three domains that encode a first peptide, a propeptide and a second peptide respectively. The result is an insulin proprotein having an internal propeptide domain. An insulin mutein is not an artificial proprotein of the present invention. However, a multimeric antibody proprotein that with one or more

Propeptide: A propeptide is a peptide that occurs between two peptides of interest in a proprotein. The propeptide is thought to assist in forming a conformational and proximational association between the two peptides of interest, which results in a stable intermediate. The two peptides of interest then form a multimeric protein.

Proprotein: A proprotein is a multimeric protein intermediate, which comprises at least three peptide sequences; a first peptide sequence of interest, an internal propeptide sequence attached to the c-terminus of the first peptide sequence of interest, and a second peptide of interest attached to the c-terminus of the propeptide sequence. The proprotein may comprise more than three peptide sequences. Any naturally occurring or non naturally occurring propeptide would conform to the present invention.

Preproprotein: A preproprotein is an arrangement of peptides having a signal peptide that precedes a proprotein in the arrangement.

Multimeric protein: A protein containing more than one polypeptide or protein where the individual polypeptides or proteins are associated with each other to form a single protein. Both heterodimeric and homodimeric proteins are multimeric proteins.

Polypeptide and peptide: A linear series of amino acid residues connected one to the other by peptide bonds between the alpha-amino and carboxy groups of adjacent residues.

Protein: A linear series of greater than about 50 amino acid residues connected one to the other as in a polypeptide.

A polypeptide or protein “domain” generally refers to a region of a polypeptide chain that is folded in such a way that confers a particular structure and/or biochemical function. (Schulz et al., supra). Domains can be defined in structural or functional terms. A functional domain can be a single structural domain, but may also include more than one structural domain. Such functions can include enzymatic catalytic activity, ligand binding, chelating of an atom or endogenous fluorescence.

“Template DNA” refers to the DNA that is amplified by “amplification primer pairs” (the population of oligonucleotide primers used in the amplification reaction). This DNA may be produced by biological (recombinant) or artificial (chemical) means. Further, mRNA may be reverse transcribed to form the template DNA that is used in the amplification reaction.

An “upstream primer” is an oligonucleotide primer, or a mixture of oligonucleotide primers, that anneal(s) to the antisense strand of the template DNA.

A “downstream primer” is an oligonucleotide primer, or a mixture of oligonucleotide primers, that anneal(s) to the sense strand of the template DNA.

“Amplifying/amplification” refers to a reaction wherein the entire template DNA, or portions thereof, are duplicated at least once, preferably many times.

“Ligating/ligation” refers to covalent coupling of two or more DNA strands (3′ end to 5′ end) using enzymatic and/or chemical methods.

A “nontemplated endonuclease recognition site” is a sequence within the nontemplated sequence that is recognized by a restriction endonuclease.

A “library” is a population of nucleic acid molecules produced using the methods described. The number of members contained in the population which differ in nucleotide sequence is determined by the number of sequences contained in the source material.

Overview of the Invention

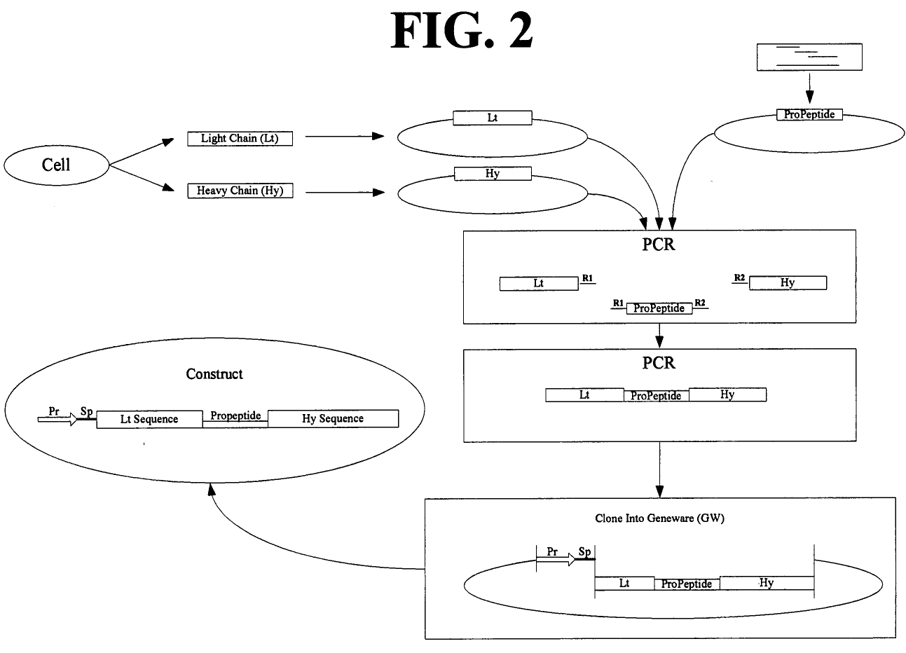

To more clearly understand the features of the present invention, an overview is provided and described with respect to FIGS. 2-11.

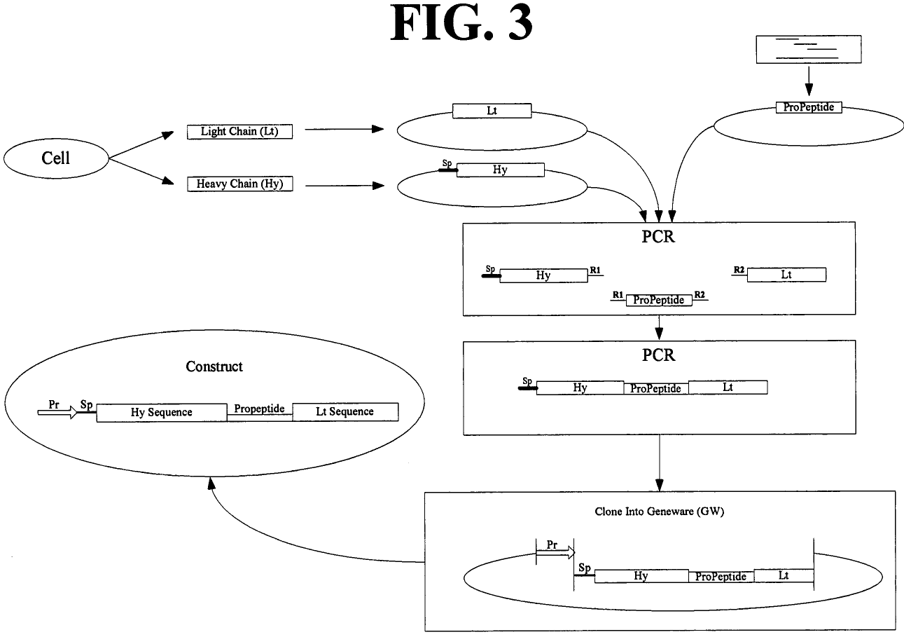

The inventors have produced numerous constructs, such as those depicted generically in FIGS. 2 and 3, for expression of desired multimeric proteins, such as antibodies and antibody fragments. Such constructs include a light chain (Lt) and a heavy chain (Hy) that have been extracted from one or more cells for a desired purpose. It should be understood that the light chain and heavy chain may be extracted from the same cell, same type of cell or completely different types of cells depending upon the desired multimeric protein subsequently expressed. Using any of a variety of known techniques, each of the light chain and heavy chain is provided with a predetermined endonuclease restriction site, such as R1 and R2 depicted in FIGS. 2 and 3. Methods for adding such restriction sites to a gene sequence are well known in the art.

A predetermined propeptide in accordance is constructed in accordance with methods described in greater detail hereinbelow (for instance, see Example A). The propeptide is further provided with R1 and R2 restrictions which are compatible ends or complementary sequences suitable for fusing with the dna fragments (light chain Lt and heavy chain Hy), as shown in FIGS. 2 and 3. As is well known, during PCR, the restriction sites enable the construction of the sequences shown in FIGS. 2 and 3 that includes the heavy chain Hy, the propeptide, and the light chain in either of the orientations depicted in FIGS. 2 and 3. Next, the Hy-propeptide-Lt sequence is cloned into, for example, a virus, such as those used in the Geneware™ system developed by Large Scale Biology Corporation, Vacaville Calif., adding thereto a signal peptide (Sp) and a promoter (Pr). After replication of the construct using Geneware™, the final construct is isolated for use in any of a variety of desired expression systems, as is described in greater detail below.

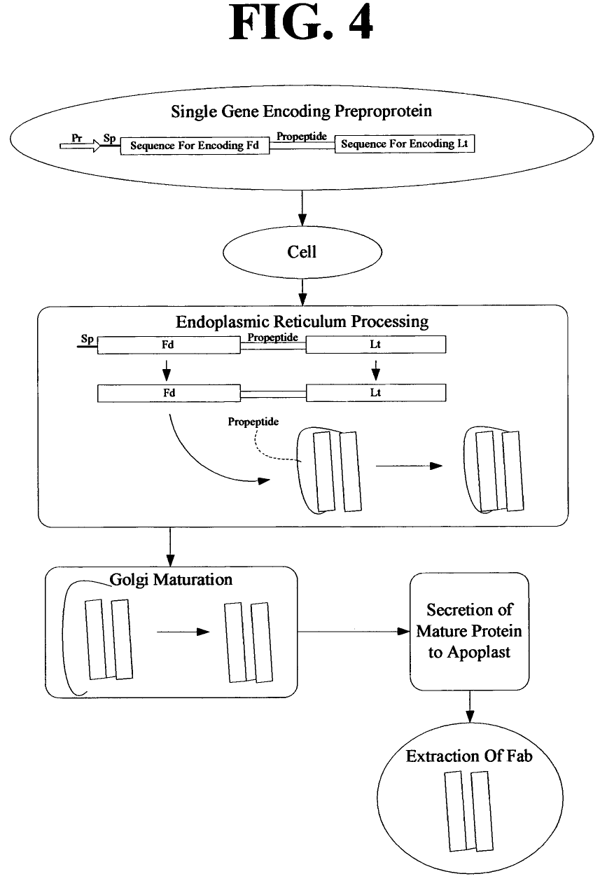

The constructs of the present invention, such as those represented generically in FIGS. 2 and 3, are inserted into cells for expression of a desired protein, proteins, antibody fragments or antibodies. These multimeric proteins may be expressed in the cell by mechanisms within the cell that are described in greater detail below with respect to FIGS. 4-9.

FIG. 4 is a block diagram representing a one embodiment of the present invention where a single gene encoding a preproprotein is introduced into a cell or cells. In this embodiment, the construct includes the promoter Pr, the signal peptide Sp, a heavy chain fragment Fd, a propeptide and a short chain Lt. In this embodiment, the short heavy chain is inserted between a signal peptide (Sp) and the propeptide. The construct is introduced into the cell, where after expression, the propeptide (Sp) is removed within the endoplasmic reticulum to produce a folded preproprotein. Subsequent maturation within the Golgi of the cell removes the propeptide thereby producing a folded desired antibody fragment or Fab, which may be extracted by any of a variety of techniques, as is described in greater detail below.

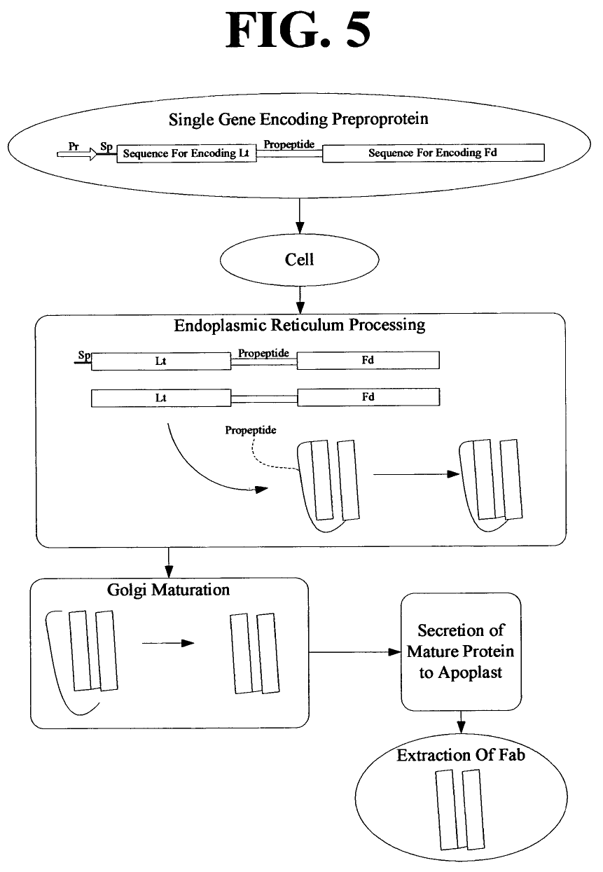

FIG. 5 is a block diagram similar to FIG. 4, except that positions of the heavy chain fragment Fd and the light chain Lt are reversed such that the construct includes the promoter Pr, the signal peptide Sp, a short chain Lt, a propeptide and a heavy chain fragment Fd. Specifically, the light chain Lt is inserted between a signal peptide (Sp) and the propeptide. The construct is introduced into the cell, where after expression, the signal peptide (Sp) is removed within the endoplasmic reticulum to produce a folded preproprotein. Subsequent maturation within the Golgi of the cell removes the propeptide thereby producing a folded desired antibody fragment or Fab, which may be extracted by any of a variety of techniques, as is described in greater detail below.

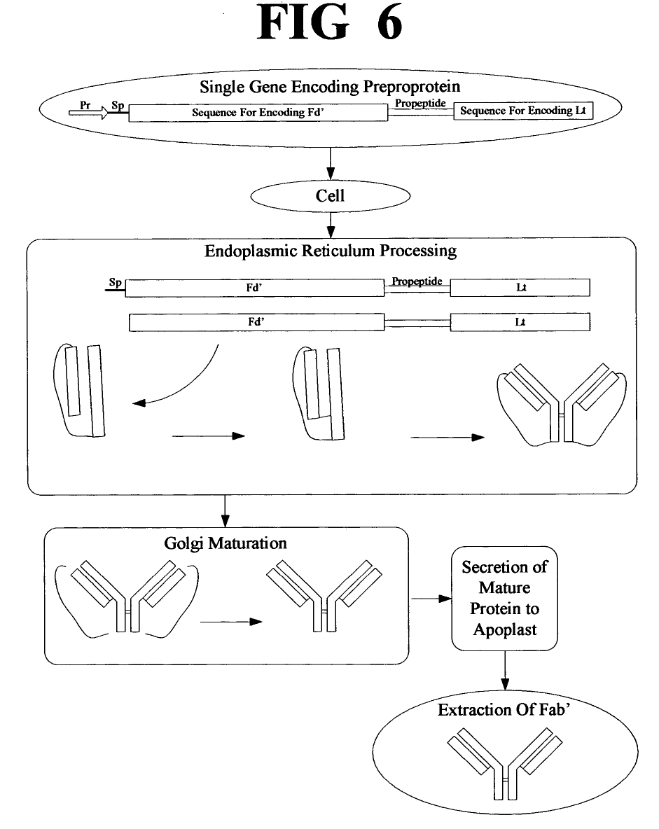

FIG. 6 is a block diagram representing another embodiment of the present invention where a single gene encoding a preproprotein is introduced into a cell or cells. In this embodiment, the construct includes the promoter Pr, the signal peptide Sp, a heavy chain fragment Fd′, a propeptide and a short chain Lt. Specifically, the heavy chain fragment Fd′ is inserted between a signal peptide (Sp) and the propeptide. The construct is introduced into the cell, where after expression, the signal peptide (Sp) is removed within the endoplasmic reticulum to produce a folded preproprotein. Subsequent maturation within the Golgi of the cell removes the propeptide thereby producing a folded desired Fab′, which may be extracted by any of a variety of techniques, as is described in greater detail below.

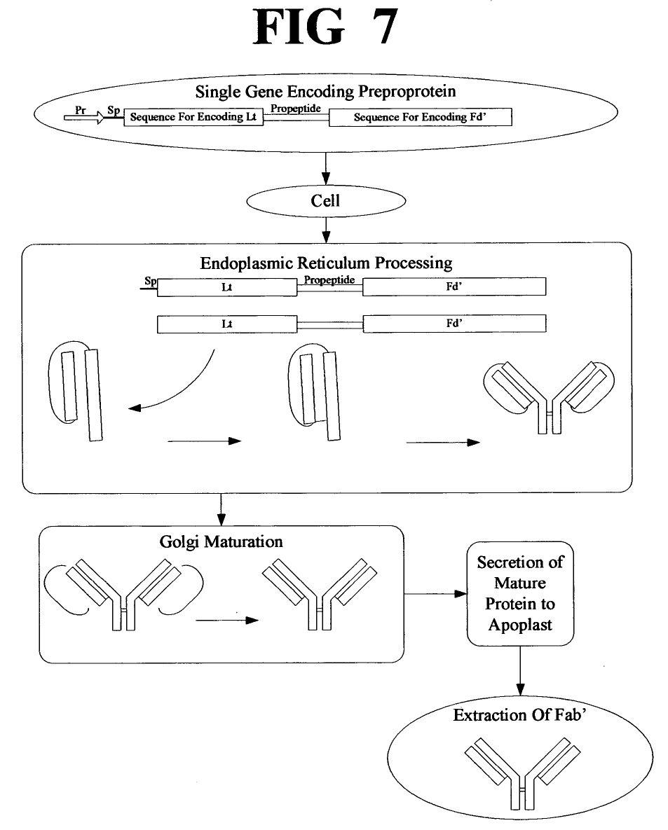

FIG. 7 is a block diagram similar to FIG. 6, except that positions of the heavy chain fragment Fd′ and the light chain Lt are reversed such that the construct includes the promoter Pr, the signal peptide Sp, a short chain Lt, a propeptide and a heavy chain fragment Fd′. Specifically, the light chain Lt is inserted between a signal peptide (Sp) and the propeptide. The construct is introduced into the cell, where after expression, the signal peptide (Sp) is removed within the endoplasmic reticulum to produce a folded preproprotein. Subsequent maturation within the Golgi of the cell removes the propeptide thereby producing a folded desired Fab′, which may be extracted by any of a variety of techniques, as is described in greater detail below.

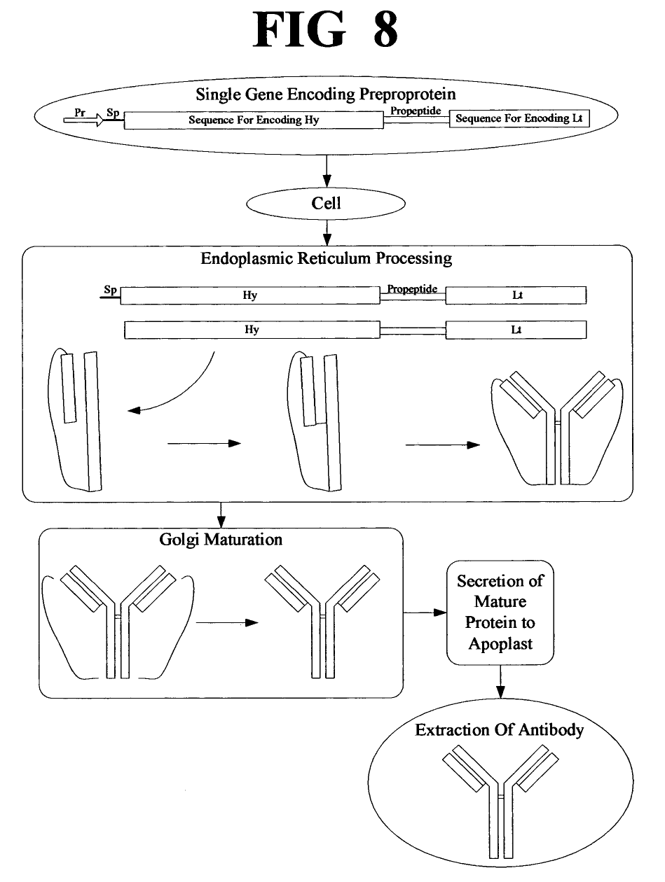

FIG. 8 is a block diagram representing yet another embodiment of the present invention where a single gene encoding a preproprotein is introduced into a cell or cells. In this embodiment, the construct includes the promoter Pr, the signal peptide Sp, a full length heavy chain Hy, a propeptide and a short chain Lt. The construct is introduced into the cell, where after expression, the signal peptide (Sp) is removed within the endoplasmic reticulum to produce a folded preproprotein. Subsequent maturation within the Golgi of the cell removes the propeptide thereby producing a folded desired antibody, which may be extracted by any of a variety of techniques, as is described in greater detail below.

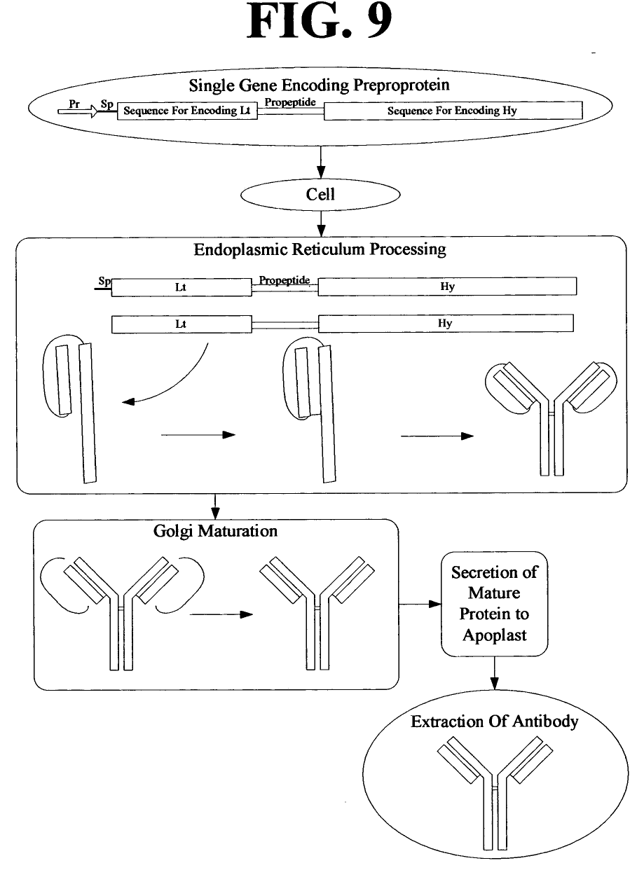

FIG. 9 is a block diagram similar to FIG. 8, except that positions of the heavy chain Hy and the light chain Lt are reversed such that the construct includes the promoter Pr, the signal peptide Sp, a short chain Lt, a propeptide and a heavy chain Hy. Specifically, the light chain Lt is inserted between a signal peptide (Sp) and the propeptide. The construct is introduced into the cell, where after expression, the signal peptide (Sp) is removed within the endoplasmic reticulum to produce a folded preproprotein. Subsequent maturation within the Golgi of the cell removes the propeptide thereby producing a folded desired antibody.

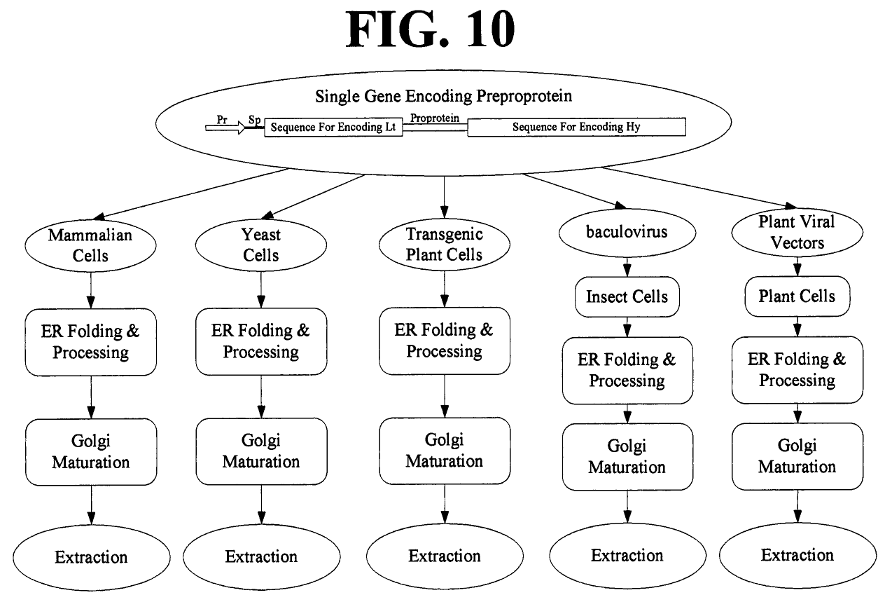

FIG. 10 is a block diagram showing various platforms that may be utilized for the production of an antibody fragment, Fab, Fab′ or a full antibody using a single gene encoding preproprotein construct in accordance the construct depicted in FIG. 2. For example, the construct of FIG. 2 may be introduced into mammalian cells, yeast cells, transgenic plant cells, baculovirus or plant viral vectors, such as those used in GenewareTM developed by Large Scale Biology Corporation.

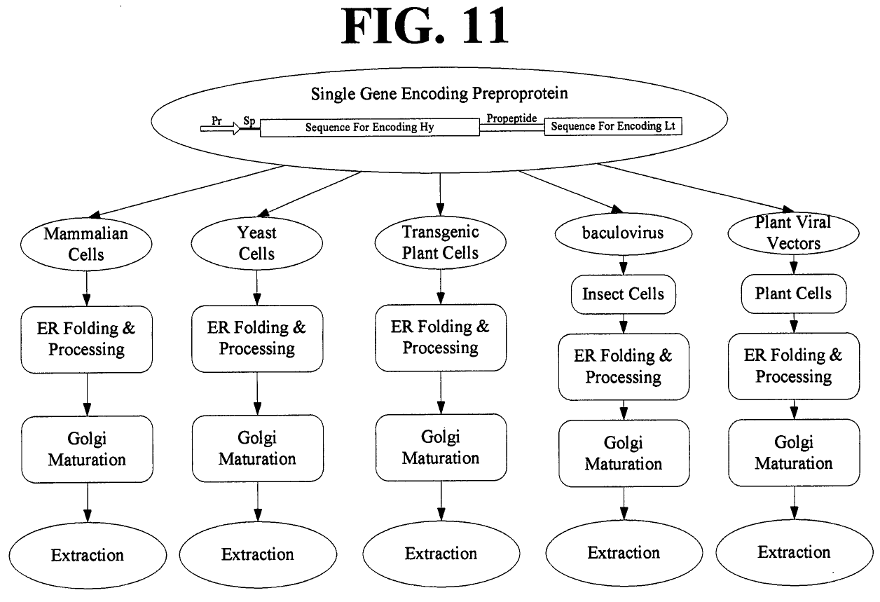

FIG. 11 is a block diagram showing various platforms that may be utilized for the production of an antibody fragment, Fab, Fab′ or a full antibody using a single gene encoding preproprotein construct in accordance the construct depicted in FIG. 3. For example, the construct of FIG. 3 may be introduced into mammalian cells, yeast cells, transgenic plant cells, baculovirus or plant viral vectors, such as those used in GenewareTM developed by Large Scale Biology Corporation.

Methods of Expressing Multimeric Proteins Using a Single Gene

The invention will first be described in its broadest overall aspects with a more detailed description following.

A class of novel, artificial proproteins has now been designed and engineered which comprise a multimeric proprotein, that is, a protein assembly capable of producing a multimeric protein from a single protein comprised of a first peptide, a second peptide and propeptide, where the first peptide and the second peptide associate to assume a biologically functional conformation essentially free of the propeptide. Examples of the first peptide and second peptide would be the light and heavy chain of an immunoglobulin molecule, the light chain and a fragment of the heavy chain immunoglobulin molecule, the alpha and beta chain of the T cell receptor, or the alpha and beta chains of hemoglobin. Examples of the propeptide would be the insulin C chain, Saccharomyces cerevisiae K1 killer toxin propeptide (gamma subunit), Kluyveromyces lactis plasmid k1 toxin propeptide and the KP6 toxin propeptide chain. This invention features an artificial, proprotein which folds to form a stable intermediate protein containing a propeptide, where the mature multimeric protein has subunits with associative properties, DNA encoding these proteins prepared by recombinant techniques, host cells harboring these DNAs, and methods for the production of these proteins and DNAs.

The design of artificial proprotein is based on the observation that multimeric proteins often have a requirement for involvement of folding chaperones to complete their complex folding and assembly requirements. The proproteins are designed to comprise a molecular chaperon in the form of a propeptide to facilitate the proper folding of multimeric proteins. The artificial proproteins are further designed to increase the availability of chaperones, increased local concentration, proper cellular localization, temporal and stochiometric expression of the protein subunits (among others) in order to increase the accumulation of the properly assembled, mature and active multimer. The propeptide influences the spatial distribution of the subunits by bringing them into close proximity, such that the relative molar concentration of each subunit is high facilitating the folding performed by BiP, PDI and other associative forces such as disulfide linkages, electrostratic and hydrophobic interactions between and within subunits.

Recombinant expression of multimeric, associative proteins is limited by the lowest subunit level and the multimer composition accumulation can be adversely influenced by inequality in subunit expression levels. The creation of a proprotein by fusing the subunit polypeptides to a stable folding and conformational propeptide which is removed by cellular mechanism results in the proper subunit interactions without being resident in the mature protein. The KP6 or other propeptide molecules act as a chaperone as described above but also may act additionally to recruit, direct and augment or catalyze the activity of other chaperones such BiP and PDI.

This invention requires recombinant production of multimeric proproteins have the ability to form a stable intermediate and be further matured to create a multimeric protein. This technology has been developed and is disclosed herein. In view of this disclosure, persons skilled in recombinant DNA technology, protein design, and protein chemistry can produce such preproproteins which will result in a biologically active mature protein.

In another embodiment, the artificial protein comprises a multimeric protein preproprotein, that is, a protein assembly capable of producing a multimeric protein from a single protein comprised of a signal peptide, a first peptide, propeptide, and a second peptide, where the first peptide and the second peptide associate to assume a biologically functional conformation essentially free of the propeptide and signal peptide. An example of the signal peptide would be the kappa light leader or the alpha amylase signal peptide.

In another embodiment of this invention, the proprotein is derived in part from a Fab fragment consisting of a portion of a immunoglobulin heavy chain and a immunoglobulin light chain. The immunoglobulin heavy chain fragment and light chains are associated with each other and assume a conformation having an antigen binding site for a predetermined or preselected antigen. The antigen binding site on a Fab fragment has a binding affinity or avidity similar to the antigen binding site on an immunoglobulin molecule.

In another embodiment, the proprotein is derived form a multimeric immunoglobulin molecule comprised of an immunoglobulin heavy chain and an immunoglobulin light chain. The immunoglobulin heavy and light chains are associated with each other and assume a conformation having an antigen binding site specific for, as evidenced by its ability to be competitively inhibited, a preselected or predetermined antigen.

In a further embodiment, the proprotein is derived from a ligand binding polypeptide (receptor) that forms a ligand binding site which specifically binds to a preselected ligand to form a complex having a sufficiently strong binding between the ligand and the ligand binding site for the complex to be isolated.

In still yet another embodiment, the proprotein is derived from a multimeric protein where that protein is an enzyme that binds to a substrate and catalyzes the formation of a product from the substrate. While the topology of the substrate binding site (ligand binding site) of the catalytic multimeric protein is probably more important for its activity than its affinity for the substrate, there is a binding requirement.

In another embodiment, novel multimeric or heterodimers would also fit in this class. Interaction of polypeptides with other polypeptides to produce stable multimeric forms not occurring in nature could be produced with this technology. This includes, naturally occurring polypeptides that do not interact as a result of production in two different organisms, organelles, or temporally or otherwise separated proteins that would interact if produced in the presence of the other. An example of such an artificial interaction would be LIN-2,7 (L27) heterodimers where each subunit is derived from different species.

The invention thus provides a family of recombinant molecules expressed form a single piece of DNA, all of which have the capacity to be processed into multiple polypeptide that have an associative property.

In a further embodiment the affinity or activity of an antibody or antibody fragment (Fab) is modified to improve desired characteristics as demonstrated in Carter, et al, (1992)Proc. Nat. Acad. Sci. vol. 89 (4285-4289). Once an antibody, whether native, chimeric or humanized with CDR exchanges, is obtained, positions in the variable heavy and light chain genes are identified as influencing the structure and function or binding of the antibody through molecular modeling comparisons of predicted structure and known crystal structures.

The identified or presumed influential positions are randomized to contain preferred amino acids for optimal structural organization as well as preferred non-immunogenic human sequences. Using any appropriate DNA shuffling method, multiple influential positions containing varied amino acids residues at any one position, are re-assorted to create a population of sequences that contain different combinations of amino acids at these influential sites.

The population of antibody sequences created by DNA shuffling are cloned as described in EXAMPLE 2 to create a population of preproprotein sequences that are cloned into viral vectors using restriction independent cohesive end cloning or another cloning method known in the art.

Infectious transcripts are generated and then encapsidated in vitro. The encapsidated transcripts are used to infect plants. Expressed proteins are subsequently harvested from the interstitial fluid or from a tissue homogenate.

The extracts are assayed for a desired activity (e.g., antigen binding) as determined by ELISA or other suitable assay. Additionally, it is preferred if the activity assay has a quantitative aspect. The samples are furthered evaluated to determine the quantity of the antibody present by ELISA or with other suitable assay.

Viral vectors containing improved antibodies can be used to inoculate larger quantities of plants to obtain purified antibody for further characterization, pre-clinical evaluation, and process development.

Concurrently, the expression system is scaled up to produce sufficiently large-scale quantities. This may involve the creation of a plant line stably transformed with the preferred proprotein or antibody encoding genes.

Methods for isolating a gene coding for a desired first polypeptide (subunit) are well known in the art. See for example, Guide To Molecular Cloning Techniques in Methods in Enzymology, Volume 152, Berger and Kimmel, eds (1987): and Current Protocols in Molecular Biology, Ausubel et al., eds., John Wiley and Sons, New York (1987) whose disclosure are herein incorporated by reference.

Genes useful in practicing this invention include genes coding for polypeptide contained in immunoglobulin products, immunoglobulin molecules, Fab fragments, enzymes, receptors, chemokines, cytokines, blood products, diagnostic, analytical and therapeutic compounds. Particularly preferred are genes coding for polypeptides that associate to form multimeric complexes.

Genes coding for a polypeptide subunit of a multimeric protein can be isolated from either the genomic DNA containing the gene expressing the polypeptide or the messenger RNA (mRNA) which codes for the polypeptide. The difficulty in using genomic DNA is in juxtaposing the sequences coding for the polypeptide where the sequences are separated by introns. The DNA fragment(s) containing the proper exons must be isolated, the introns excised, and the exons spliced together in the proper order and orientation. For the most part, this will be difficult so the alternative technique employing mRNA will be the method of choice because the sequence is contiguous (free of introns) for the entire polypeptide. Methods for isolating mRNA coding for peptides or proteins are well known in the art. See, for example, Current Protocols in Molecular Biology, Ausubel et al., John Wiley and Sons, New York (1987), Guide to Molecular Cloning Techniques, in Methods In Enzymology, Volume 152, Berger and Kimmel, eds. (1987), and Molecular Cloning: A Laboratory Manual, Maniatis et al., eds., Cold Spring Harbor Laboratory, Cold Spring Harbor, N.Y. (1982).

The polypeptide coding genes isolated above are assembled into a proprotein and typically operatively linked to an expression vector. Expression vectors compatible with the host cells are used to express the genes of the present invention. Typical expression vectors useful for expression of genes in various hosts are well known in the art and include vectors derived from with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

The expression vectors described above contain expression control elements including the promoter. The polypeptide coding genes are operatively linked to the expression vector to allow the promoter sequence to direct RNA polymerase binding and synthesis of the desired polypeptide coding gene. Useful in expressing the polypeptide coding gene are promoters which are inducible, viral, synthetic, constitutive, temporally regulated, spatially regulated, and spatiotemporally regulated. The choice of which expression vector and ultimately to which promoter a polypeptide coding gene is operatively linked depends directly, as is well known in the art, on the functional properties desired, e.g. the location and timing of protein expression, and the host cell to be transformed, these being limitations inherent in the art of constructing recombinant DNA molecules. However, an expression vector useful in practicing the present invention is at least capable of directing the replication, and preferably also the expression of the polypeptide coding gene included in the DNA segment to which it is operatively linked.

Preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May 1993)); and hygro, which confers resistance to hygromycin (Santerre et al., 1984, Gene 30:147). Methods commonly known in the art of recombinant DNA technology which can be used are described in Ausubel et al., eds., Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al., eds, Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, “The use of vectors based on gene amplification for the expression of cloned genes in mammalian cells,” in DNA Cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

Expression of the desired multimeric protein can be identified by assaying for the presence of the biologically multimeric protein using assay methods well known in the art. Such methods include Western blotting, immunoassays, binding assays, and any assay designed to detect a biologically functional multimeric protein. See, for example, the assays described in Immunology: The Science of Self-Nonself Discrimination, Klein, John Wiley and Sons, New York, N.Y. (1982).

Preferred screening assays are those where the biologically active site on the multimeric protein is detected in such a way as to produce a detectible signal. This signal may be produced directly or indirectly and such signals include, for example, the production of a complex, formation of a catalytic reaction product, the release or uptake of energy, and the like. For example, a host containing an antibody molecule produced by this method may be processed in such a way to allow that antibody to bind its antigen in a standard immunoassay such as an ELISA or a radio-immunoassay similar to the immunoassays described in Antibodies: A Laboratory Manual, Harlow and Lane, eds., Cold Spring Harbor Laboratory, Cold Spring Harbor, N.Y. (1988).

A further aspect of the present invention is a method of producing a proprotein comprised of a first and a second polypeptide and a propeptide. Generally, the method combines the elements of propagating or culturing a host of the present invention, and harvesting the host cell or cells that was cultivated to produce the desired multimeric protein.

The host of the present invention containing the desired multimeric protein precursor comprised of a first polypeptide and a second polypeptide and a propeptide is propagated or cultured using methods well known to one skilled in the art. Any of the recombinant hosts of the present invention may be cultured or propagated to isolate the desired multimeric protein they contain.

After culture, the recombinant host is harvested to recover the produced multimeric protein. This harvesting step may consist of harvesting the entire host, or isolating specific organelles or extracts such as the media or secreted fraction which facilitate further purification.

In preferred embodiments this harvesting step further comprises the steps of:

(a) harvesting the secreted fraction from host to produce a multimeric protein containing solution; and

(b) isolating said multimeric protein from said solution.

In another embodiment this harvesting step further comprises the steps of:

(a) homogenizing at least a portion of host;

(b) extracting said multimeric protein from said homogenate to produce a multimeric protein containing solution; and

(c) isolating said multimeric protein from said solution.

The multimeric protein is isolated from the solution produced above using methods that are well known to those skilled in the art of protein isolation. These methods include, but are not limited to, immuno-affinity purification and purification procedures based on the specific size, electrophoretic mobility, biological activity, and/or net charge of the multimeric protein to be isolated.

The contemplated recombinant hosts contain a multimeric protein. This multimeric protein may be an immunoglobulin product described above, an enzyme, a receptor capable of binding a specific ligand, or an abzyme.

An enzyme of the present invention is a proprotein derived at least two polypeptide chains. This proprotein is encoded by a gene introduced into the recombinant host by the method of the present invention. Useful enzymes include aspartate transcarbamylase and the like.

In another preferred embodiment the proprotein is derived from a receptor capable of binding a specific ligand. Typically this receptor is made up of a proprotein encoded by a gene introduced into the recombinant host by a method of the present invention. Examples of such receptors and their respective ligands include hemoglobin, O.sub.2 ; protein kinases, cAMP; and the like.

In another preferred embodiment of the present invention the immunoglobulin product present is an abzyme constituted by either an immunoglobulin heavy chain and its associated variable region, or by an immunoglobulin heavy chain and an immunoglobulin light chain associated together to form an immunoglobulin molecule, a Fab or a substantial portion of an immunoglobulin molecule. Illustrative abzymes include those described by Tramontano et al., Science, 234: 1566-1570 (1986): Pollack et al., Science, 234: 1570-1573 (1986): Janda et al., Science, 241: 1188-1191 (1988); and Janda et at., Science, 244: 437-440 (1989).

Typically, proproteins of the present invention contain at least two polypeptides and the propeptide; however, more than two peptides can also be present. Each of these polypeptides is separated by a propeptide such that they fold and are processed into a multimeric protein. The polypeptide subunits are associated with one another to form a multimeric protein by disulfide bridges, by hydrogen bonding, or like mechanisms.

There are numerous examples of multimeric proteins that could be made in this way. The following list comprises several multimeric proteins that are not naturally made with a propeptide. The list is intended to be exemplary. Several other multimeric proteins exist that are not made with a propeptide. All such multimeric proteins, if made using a propeptide would conform to the present invention. Examples are hemoglobin (α₂β₂), IL-12 (p35 and p40), TCR, MHC class II heterodimer (αβ), CD8 heterodimer (αβ),CD3 (εδ), CD3 (εγ), CD22(αβ), CD41(GPIIba CD61) Janus kinase(JAK), JAK and STAT (signal transducers and activators of transcription) heterodimers, IgM heavy chain with I chain, or VpreB and lambda 5 (I chain), Igβ and Igα, Integrins such as T-cell integrin LFA-1 (α_{L}β₂), CD152(CTLA-4), IL-2 receptor(heterotrimer) IL-2R(αβγc), IL-15(αβγ), Rhematopoietin receptor family (IL-3R, GM-CSFR are a few), TNF-β (LT-α and LT-β), IL12R(β1β2), IgM (H₂L₂) with transgenic J chain, IgA (H₂L₂) with transgenic J chain, MHC class I (α and β₂-microglobulin), HLA-DM(αβ), mouse H-2M(αβ), E.coli DNA polymerase III, insulin receptor(IR) (α₂β₂), IGF-1 receptor(α₂β₂), G proteins heterotrimers (αβ) such as adrenergic receptor, retinoic acid receptor (RAR) (αβ), oestrogen receptor(αβ), myocyte enhancer factors 2 (MEF2) family such as c-fos and JunD, yeast RNAPII Rpb3/Rpb11 heterodimer, calpain, importin alpha2/beta heterodimer, DNA-dependent protein kinase (DNA-PKcs, and Ku70 and Ku80), Ku70 and Ku80 heterodimer, Hepatopoietin (HPO) and HPO23 heterodimer, leukocyte function associated antigen-1 molecule (LFA-1) CD11a (alphaL) and CD18 (beta2) integrin subunit heterodimer, liver X receptor (LXR)/retinoid X receptor (RXR) heterodimer, eukaryotic structural maintenance of chromosome (SMC) proteins, human mismatch repair (MMR) heterodimers, rBAT-b(0,+)AT heterodimer, retinoid X alpha (RXRalpha) and peroxisome proliferator-activated receptor alpha (PPARalpha) heterodimer, thyroid hormone receptor (TR)/RXR heterodimer, peroxisome proliferator activated receptor/RXR, Nurr1 orphan nuclear receptor/RXR heterodimer, calcineurin, Collapsin response mediator protein-2 and tubulin heterodimer, CD94/NKG2A heterodimer, IkappaB kinase complex, human immunodeficiency virus reverse transcriptase (RT) heterodimer, CD98 complex, B cell antigen receptor with the membrane-bound immunoglobulin molecule (mIg) and the Ig-alpha/Ig-beta heterodimer, class IA phosphoinositide 3-kinase, hypoxia inducible factor 1, as well as others obvious to those skilled in the art.

It is preferred to remove the propeptide to obtain the mature multimeric protein, essentially free of foreign sequences which are potentially destabilizing or could interfere with the active site or antigen binding region and potentially be adversely immunogenic. It may be beneficial to engineer the proprotein such that small foreign regions remain after the removal of the propeptide sequence such that these additional sequences would be useful for purification or confer other biological function such as immuno-regulation. Often, a few amino acid spacer is inserted between the polypeptide domains and is designed to transition from one domain to another. In a preferred embodiment a di-glycine spacer functions to buffer the joint of the heterologous polypeptides and facilitate proper folding of juxtaposed domains and minimize and enhance the transition of one domain to another. Other amino acids may be used to further improve the folding and chaperone activity of the propeptide, further optimizing the propeptide folding.

Novel multimeric proteins which have polypeptide subunits with associative properties but are not naturally found associated would also fit in this class. Interaction of proteins with other proteins to produce stable multimeric forms not occurring in nature could be produced with this technology. Additionally, naturally occurring proteins that do not interact as a result of production in two different organisms, organelles, are temporally or otherwise separated proteins that would interact if produced in the presence of the other. An example of such an artificial interaction would be LIN-2,7 (L27) heterodimers where each subunit is derived from different species.

Cloning of Domains

A domain may be isolated by any of a number of techniques. In general, a nucleic acid sequence encoding a polypeptide (or RNA) domain of interest is cloned from an appropriate cDNA library or a genomic DNA library based on hybridization with a oligonucleotide probe that represents the domain.

For the present invention, preferred nucleic acids and proteins are mammalian, more preferably human sequences.

Alternatively, the DNA is isolated by amplification techniques using oligonucleotide primers starting with a DNA or RNA template. (See, e.g., Dieffenfach et al., PCR Primer: A Laboratory Manual (1995)). These primers can be used to amplify either a full length coding sequence or a partial sequence that could constitute a probe (ranging in length up to about several thousand nucleotides). The resultant probe sequence is then used to screen a mammalian library for the full-length nucleic acid of interest. Use of synthetic oligonucleotide primers and amplification of an RNA or DNA template is described in U.S. Pat. Nos. 4,683,195 and 4,683,202; PCR Protocols: A Guide to Methods and Applications (Innis et al., eds, 1990)). Methods such as PCR and ligase chain reaction (LCR) can be used to amplify nucleic acid sequences of domains directly from mRNA, from cDNA, or from genomic or cDNA libraries. Degenerate oligonucleotides can be designed to amplify domain homologues using the known sequences that encode the domain. Restriction endonuclease sites can be incorporated into the primers. Genes amplified by the PCR reaction can be purified on agarose gels and cloned into an appropriate vector.

In expression cloning, nucleic acids are isolated from expression libraries using as a probe an antibody (or other binding partner) specific for an epitope of the expressed polypeptide. Polyclonal or monoclonal antibodies (mAbs) can be raised by immunization with one or more peptide fragments of the domain being cloned.

Nucleic acid probes, preferably oligonucleotides are used under preferably stringent hybridization conditions to screen libraries in order to isolate polymorphic variants or alleles of the genes that encode the polypeptide domain of interest. Alternatively, antibody-based expression cloning permits cloning of polymorphic or allelic variants or interspecies homologues.

Selection of sources for the cDNA library and its production from mRNA is done using conventional methods (Gubler et al., Gene 25:263-269 (1983); Sambrook et al., Molecular Cloning, A Laboratory Manual (2^{nd} ed. 1989); Current Protocols in Molecular Biology (Ausubel et al., eds., 1994 or latest edition).

Methods for preparing genomic DNA libraries are conventional in the art. For example, DNA extracted from a tissue may be mechanically sheared or enzymatically digested to yield fragments of about 12-20 kb that are separated by gradient centrifugation and inserted into appropriate expression vectors. These vectors are packaged into phage in vitro. Recombinant phage are analyzed by plaque hybridization (Benton et al., Science 196:180-182 (1977). Colony hybridization is carried out, for example, as generally described by Grunstein et al., Proc. Natl. Acad. Sci. USA., 72:3961-3965 (1975).

Synthetic oligonucleotides can be used to construct recombinant “genes” for use as probes or for expression of the domain polypeptides.

Oligonucleotides can be chemically synthesized using solid phase phosphoramidite triester methods (Beaucage et al., Tetrahedron Letts. 22:1859-1862 (1981)) using an automated synthesizer (Van Devanter et al., Nucleic Acids Res. 12:6159-6168 (1984)). Purification of oligonucleotides is typically by native acrylamide gel electrophoresis or by anion-exchange HPLC (Pearson et al., J. Chrom. 255:137-149 (1983)).

Sequences of cloned genes and synthetic oligonucleotides can be verified by conventional methods such as the chain termination method (Wallace et al., Gene 16:21-26 (1981) using a series of overlapping oligonucleotides usually 40-120 bp in length, representing both the sense and antisense strands of the gene.

The nucleic acid encoding the desired polypeptide is typically cloned into an intermediate vector before transformation or transfection of prokaryotic or eukaryotic cells for replication and/or expression of the nucleic acid. These intermediate vectors, e.g., plasmids or shuttle vectors, are typically for use in prokaryotic cells.

Expression System for Production of Multimeric Proteins

A number of well-known heterologous expression systems in bacterial, insect, mammalian and plant were discussed above, each with its advantages and disadvantages. The present invention is particularly suited for plant expression.

A number of transformation methods permit expression of heterologous proteins in plants. Some involve the construction of a transgenic plant by integrating DNA sequences encoding the protein of interest into the plant genome. The time it takes to obtain transgenic plants may be too long for the rapid production certain embodiments such as a tumor vaccine polypeptide. An attractive solution (an alternative to such stable transformation) is transient transfection of plants with expression vectors. Both viral and non-viral vectors capable of such transient expression are available (Kumagai, M. H. et al. (1993) Proc. Nat. Acad. Sci. USA 90:427-430; Shivprasad, S. et al. (1999) Virology 255:312-323; Turpen, T. H. et al. (1995) BioTechnology 13:53-57; Pietrzak, M. et al. (1986) Nucleic Acid Re. 14:5857-5868; Hooykaas, P. J. J. and Schilperoort, R. A. (1992) Plant Mol. Biol. 19:15-38), although viral vectors are easier to introduce into host cells, spread by infection to amplify the expression and are therefore preferred.

Chimeric genes, vectors and recombinant viral nucleic acids of this invention are constructed using conventional techniques of molecular biology. A viral vector that expresses heterologous proteins in plants preferably includes (1) a native viral subgenomic promoter (Dawson, W. O. et al. (1988)Phytopathology 78:783-789 and French, R. et al. (1986) Science 231:1294-1297), (2) preferably, one or more non-native viral subgenomic promoters (Donson, J. et al. (1991) Proc. Nat. Acad. Sci. USA 88:7204-7208 and Kumagai, M. H. et al. (1993) Proc. Nat. Acad. Sci. USA 90:427-430), (3) a sequence encoding viral coat protein (native or not), and (4) nucleic acid encoding the desired heterologous protein. Vectors that include only non-native subgenomic promoters may also be used. The minimal requirement for the present vector is the combination of a replicase gene and the coding sequence that is to be expressed, driven by a native or non-native subgenomic promoter. The viral replicase is expressed from the viral genome and is required to replicate extrachromosomally. The subgenomic promoters allow the expression of the foreign or heterologous coding sequence and any other useful genes such as those encoding viral proteins that facilitate viral replication, proteins required for movement, capsid proteins, etc. The viral vectors are encapsidated by the encoded viral coat proteins, yielding a recombinant plant virus. This recombinant virus is used to infect appropriate host plants. The recombinant viral nucleic acid can thus replicate, spread systemically in the host plant and direct RNA and protein synthesis to yield the desired heterologous protein in the plant. In addition, the recombinant vector maintains the non-viral heterologous coding sequence and control elements for periods sufficient for desired expression of this coding sequence.

The recombinant viral nucleic acid is prepared from the nucleic acid of any suitable plant virus, though members of the tobamovirus family are preferred. The native viral nucleotide sequences may be modified by known techniques providing that the necessary biological functions of the viral nucleic acid (replication, transcription, etc.) are preserved. As noted, one or more subgenomic promoters may be inserted. These are capable of regulating expression of the adjacent heterologous coding sequences in infected or transfected plant host. Native viral coat protein may be encoded by this RNA, or this coat protein sequence may be deleted and replaced by a sequence encoding a coat protein of a different plant virus (“non-native” or “foreign viral”). A foreign viral coat protein gene may be placed under the control of either a native or a non-native subgenomic promoter. The foreign viral coat protein should be capable of encapsidating the recombinant viral nucleic acid to produce functional, infectious virions. In a preferred embodiment, the coat protein is foreign viral coat protein encoded by a nucleic acid sequence that is placed adjacent to either a native viral promoter or a non-native subgenomic promoter. Preferably, the nucleic acid encoding the heterologous protein, e.g., an immunogenic polypeptide to be expressed in the plant, is placed under the control of a native subgenomic promoter.

An important element of this invention, that is responsible in part for the proper folding and copious production of the heterologous protein is the presence of a signal peptide sequence that directs the newly synthesized protein to the plant secretory pathway. The sequence encoding the signal peptide is fused in frame with the DNA encoding the polypeptide to be expressed. A preferred signal peptide is the α-amylase signal peptide.

In another embodiment, a sequence encoding a movement protein is also incorporated into the viral vector because movement proteins promote rapid cell-to-cell movement of the virus in the plant, facilitating systemic infection of the entire plant.

Either RNA or DNA plant viruses are suitable for use as expression vectors. The DNA or RNA may be single- or double-stranded. Single-stranded RNA viruses preferably may have a plus strand, though a minus strand RNA virus is also intended.

The recombinant viral nucleic acid is prepared by cloning in an appropriate production cell. Conventional cloning techniques (for both DNA and RNA) are well known. For example, with a DNA virus, an origin of replication compatible with the production cell may be spliced to the viral DNA.

With an RNA virus, a full-length DNA copy of the viral genome is first prepared by conventional procedures: for example, the viral RNA is reverse transcribed to form +subgenomic pieces of DNA which are rendered double-stranded using DNA polymerases. The DNA is cloned into an appropriate vector and inserted into a production cell. The DNA pieces are mapped and combined in proper sequence to produce a full-length DNA copy of the viral genome. Subgenomic promoter sequences (DNA) with or without a coat protein gene, are inserted into nonessential sites of the viral nucleic acid as described herein. Non-essential sites are those that do not affect the biological properties of the viral nucleic acid or the assembled plant virion. cDNA complementary to the viral RNA is placed under control of a suitable promoter so that (recombinant) viral RNA is produced in the production cell. If the RNA must be capped for infectivity, this is done by conventional techniques.

Examples of suitable promoters include the lac, lacuv5, trp, tac, lp1 and ompF promoters. A preferred promoter is the phage SP6 promoter or T₇ RNA polymerase promoter.

Production cells can be prokaryotic or eukaryotic and include Escherichia coli, yeast, plant and mammalian cells.

Numerous plant viral vectors are available and well known in the art (Grierson, D. et al. (1984) Plant Molecular Biology, Blackie, London, pp.126-146; Gluzman, Y. et al. (1988) Communications in Molecular Biology: Viral Vectors, Cold Spring Harbor Laboratory, New York, pp. 172-189). The viral vector and its control elements must obviously be compatible with the plant host to be infected. Suitable viruses are

(a) those from the tobacco mosaic virus (TMV) group, such as TMV, tobacco mild green mosaic virus (TMGMV), cowpea mosaic virus (CMV), alfalfa mosaic virus (AMV), Cucumber green mottle mosaic virus—watermelon strain (CGMMV-W), oat mosaic virus (OMV),

(b) viruses from the brome mosaic virus (BMV) group, such as BMV, broad bean mottle virus and cowpea chlorotic mottle virus,

(c) other viruses such as rice necrosis virus (RNV), geminiviruses such as Tomato Golden Mosaic virus (TGMV), Cassava Latent virus (CLV) and Maize Streak virus (MSV).

A preferred host is Nicotiana benthamiana. The host plant, as the term is used here, may be a whole plant, a plant cell, a leaf, a root shoot, a flower or any other plant part. The plant or plant cell is grown using conventional methods.

A preferred viral vector for use with N. benthamiana is a modified TTO1A vector containing a hybrid fusion of TMV and tomato mosaic virus (ToMV) (Kumagai, M H. et al. (1995) Proc. Natl. Acad. Sci. USA 92:1679-1683). The inserted subgenomic promoters must be compatible with TMV nucleic acid and capable of directing transcription of properly situated (e.g., adjacent) nucleic acids sequences in the infected plant. The coat protein should permit the virus to systemically infect the plant host. TMV coat protein promotes systemic infection of N. benthamiana.

Infection of the plant with the recombinant viral vector is accomplished using a number of conventional techniques known to promote infection. These include, but are not limited to, leaf abrasion, abrasion in solution and high velocity water spray. The viral vector can be delivered by hand, mechanically or by high pressure spray of single leaves.

Purification of the Protein/Polypeptide Product

The multimeric protein produced is preferably recovered and purified using standard techniques. Suitable methods include homogenizing or grinding the plant or the producing plant parts in liquid nitrogen followed by extraction of protein. If for some reason it is not desirable to homogenize the plant material, the polypeptide can be removed by vacuum infiltration and centrifugation followed by sterile filtration. Protein yield may be estimated by any acceptable technique. Polypeptides are purified according to size, isoelectric point or other physical property. Following isolation of the total secreted proteins from the plant material, further purification steps may be performed. Immunological methods such as immunoprecipitation or, preferably, affinity chromatography, with antibodies specific for epitopes of the desired polypeptide may be used.

Various solid supports may be used in the present methods: agarose®, Sephadex®, derivatives of cellulose or other polymers. For example, staphylococcal protein A (or protein L) immobilized to Sepharose® can be used to isolate the target protein by first incubating the protein with specific antibodies in solution and contacting the mixture with the immobilized protein A which binds and retains the antibody-target protein complex.

Using any of the foregoing or other well-known methods, the polypeptide is purified from the plant material to a purity of greater than about 50%, more preferably greater than about 75%, even more preferably greater than about 95%.

Determination of Correct Folding

Critical for certain properties such as antigen recognition or ligand binding is the protein's conformation in solution. The conformation of the relevant domains of the multimeric polypeptide in solution preferably resemble that of the native protein or proteins. By producing polypeptides in plants, and targeting them to the plant's secretory pathway, the present invention insures that the polypeptide is secreted in soluble form.

A preferred reagent to be used in determining proper folding is a specific ligand, preferably an antigen, which (1) is bound by the multimeric protein when the chains are correctly folded but (2) does not bind when the chains are denatured. The antigen is employed in any of a number of immunological assays, including dot blot, western blot, immunoprecipitation, radioimmunoassay (RIA), and enzyme immunoassays (EIA) such as an enzyme-linked immunosorbent assays (ELISA). In preferred embodiments, when such antigens are available, Western blots and ELISAs are employed to verify correct folding of the relevant parts of the multimeric polypeptide produced in the plant.

Additional Analysis of the Multimeric Protein

DNA encoding the proprotein can be sequenced, yielding a deduced amino acid sequence of its encoded product. If the DNA molecule has been subcloned, it can be excised from the vector with a restriction enzyme and the resulting fragments analyzed on agarose gels to determine the size of the fragments.

A DNA molecule encoding a proprotein is first expressed. If desired, the DNA can be additionally modified to include sequences that will permit or optimize expression in an appropriate host or in an in vitro transcription/translation system. Once expressed, the multimeric polypeptide is then subjected to appropriate functional assays, e.g., measurement of enzymatic activity (of either domain). Also the quantity and physical properties of the multimeric polypeptide can be determined, e.g., by SDS-PAGE. If a domain has binding activity, or other functions as have been described above, this can also be measured by conventional means.

Other methods to improve on the propeptide activity by design and selective processes are envisioned.

Having now generally described the invention, the same will be more readily understood through reference to the following examples which are provided by way of illustration, and are not intended to be limiting of the present invention, unless specified.

The following examples are provided by way of illustration only and not by way of limitation. Those of skill will readily recognize a variety of noncritical parameters which could be changed or modified to yield essentially similar results.

### EXAMPLE 1

Cloning of the UmV KP6 Propeptide

The UmV KP6 propeptide region containing amino acids 106-138 was codon optimized for viral expression and assembled using overlapping synthetic oligonucleotides. Three overlapping oligonucleotides, one upstream, KP6-5′ (Seq ID No: 33), and two downstream, KP6-c3′ (Seq ID No: 34) and Kp6-3′ (Seq ID No: 35), were designed to have adenosine or thymidine preferentially in the third or wobble position for each triplet codon. A 100 μL PCR reaction containing 0.2 μM KP6-5′, 0.2 μM KP6-c3′, 0.2 μM Kp6-3′, 1×Cloned Pfu Buffer, 0.1 mM dATP, 0.1 mM dCTP, 0.1 mM dGTP, 0.1 mM dTTP, 1.25 Units Cloned Pfu Polymerase enzyme. The PCR reaction was amplified at 94° C. for 30 seconds, 25 cycles of 94° C. for 10 seconds, 48° C. for 15 seconds, 72° C. for 15 seconds, and 7 minutes at 72° C. The product from the above reaction was subsequently amplified with flanking primers which incorporates the coding sequence of a diglycine spacer at the 5′ end and KP6 toxin amino acids 139-141 and a diglycine spacer to the 3′ end of the synthetic KP6 propeptide sequence. A 100 μL PCR reaction containing 1 μM 5228 (Seq ID No: 36); 1 μM 5229 (Seq ID No: 37), 0.75×Cloned Pfu Buffer, 0.2 mM dATP, 0.2 mM dCTP, 0.2 mM dGTP, 0.2 mM dTTP, 1.25 Units Cloned Pfu Polymerase, 25 μL of the above PCR reaction and water used to bring the reaction to 100μL. The PCR reaction was amplified at 94° C. for 1 minute, 25 cycles of 94° C. for 30 seconds, 55° C. for 30 seconds, 72° C. for 30 seconds, and 7 minutes at 72° C. The amplification of the desired approximately 120 bp KP6 propeptide encoding sequence was confirmed by agarose gel electrophoresis. The PCR fragment from the above reaction was cloned into pCR4Blunt-TOPO (Invitrogen) following the manufacturers directions to create plasmid pLSBC1731 (Seq ID No: 75). Briefly, 1 μL of PCR product, 1 μL vector, 1 μL of salt solution and 3 μL of water were mixed, incubated at room temperature for 5 minutes. The ligation was placed on ice and 25 μL of chemically competent Top 10 cells was added to the ligation and the mix was incubated on ice for 10 minutes. The transformation reaction was heat shocked by incubating at 42° C. for 30 seconds and immediately placed on ice and 250 μL of SOC was added. The transformation was allowed to recover by incubating at 37° C., 200 rpm shaking for 20 minutes. The transformation was plated out on LB plates containing ampicillin and grown overnight at 37° C. Individual colonies were used to inoculate 1.0 mL Super Broth (SB) containing 100 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.). Briefly, the cells were pelleted by centrifugation at 3 K rpm for 15 minutes in a plate centrifuge. The supernatant was drained from the cell pellets and the cells resuspended in 250 μL P1 Buffer by vortexing. 250 μL of P2 was added to the cells, mixed by inverting and incubated for 5 minutes to lyse the cells. 350 μL of N3 was added to the cell lysates, mixed by inverting and, transferred to the Turbo Filter plate. A vacuum was applied to the Turbo Filter, which filtered the sample into the QIAprep plate. A vacuum was then applied to the QIAprep plate pulling the sample through the plate and bound the plasmid to the plate membrane. The QIAprep plate was washed using vacuum force with 0.9 mL of PB, followed by two washes with 0.9 mL of PE and vacuum dried. 100 μL EB buffer was added to the purified plasmid, incubated for 1 minute, and subsequently centrifuged for 3 minute at 6K rpm to elute the purified plasmid. The purified pLSBC1731 (Seq ID No: 75) plasmid was subjected to nucleic acid sequencing using standard methods to verify the KP6 propeptide sequence.

### EXAMPLE 2

Cloning of the Human Fab PREPROPROTEIN Library and Expression Analysis

Messenger RNA (mRNA) enriched for sequences containing long poly A tracts was isolated from total human spleen RNA (Clontech, Palo Alto, Calif.) using Dynabeads Oligo (dT)₂₅ (Dynal, Oslo, Norway). The RNA was pelleted by centrifugation at 15 K rpm, 4° C. for 15 minutes, the supernatant removed and 1 mL of 70% ethanol added. The sample was centrifuged at 15 K rpm, 4° C. for 15 minutes, the supernatant removed and the pellet resuspended in 150 μL nuclease free water (Ambion, Austin, Tex.). 5 μg of the above prepared total RNA was incubated at 65° C. for 2 minutes, immediately placed on ice for 3 minutes, and then applied to 20 μL of magnetic beads in binding buffer (20 mM Tris-HCl (pH 7.5), 1.0 M LiCl, 2 mM EDTA) where the beads were prepared by washing with 50 μL of binding buffer. The RNA and bead mixture were incubated for 5 minutes with constant rotating. The supernatant containing unbound material was removed and the beads were washed with 100 μL washing buffer (10 mM Tris-HCl (pH 7.5), 0.15 M LiCl, 1 mM EDTA) followed by the addition of 40 μL nuclease free water. Complementary DNA (cDNA) was synthesized in 60 μL reactions containing 50 mM Tris HCl (pH 8.3), 75 mM KCl, 3 mM MgCl₂, 10 mM DTT, 2 Units RNasin (Promega, Madison, Wis.), 20 Units Superscript II (Invitrogen, Carlsbad, Calif.), 0.5 mM dATP, 0.5 mM dCTP, 0.5 mM dGTP, 0.5 mM dTTP, and the oligo dT bound RNA from above. The cDNA reaction was incubated at 42° C. for 60 minutes with constant rotation. Separate PCR reactions were set up as follows to amplify the gamma VH3 heavy chain Fd (V_{H}-C_{H}1) regions or the kappa 1 light chains (V_{L}-C_{L}) including the kappa leader from the synthesized cDNA. The 100 μL PCR reactions contained 1×Taq Reaction buffer with MgCl₂, 0.2 mM dATP, 0.2 mM dCTP, 0.2 mM dGTP, 0.2 mM dTTP, 10 Units Taq Polymerase (Stratagene, La Jolla, Calif.) 1μM upstream primer, 1μM downstream primer and 1 μL prepared cDNA. To amplify the kappa 1 leader and light chain cDNAs, the reaction contained the 5230 (Seq ID No: 29) upstream and 5235 downstream primers. The 5230 upstream primer was designed to amplify approximately 13 of the 16 different kappa 1 V-gene segments including the leader sequences. The 5230 primer incorporated a Pac I site upstream of the translation start site for subsequent cloning. The 5235 downstream primer anneals to the 3′ end of the kappa C_{L} ORF, removing the termination codon, incorporates the coding sequence for a diglycine spacer fused to the 5′ end of the KP6 propeptide coding sequence. To amplify the VH3 heavy chain gamma C_{H}1 cDNAs, the reaction contained the 5236 (Seq ID No: 32) upstream and 5233 (Seq ID No: 30) downstream primers. The 5236 upstream primer was designed to amplify approximately 14 of the 18 different VH3 V-gene segments with out the leader sequence. The 5236 primer incorporates the coding sequence for a diglycine spacer fused to the 3′ end of the KP6 propeptide coding sequence. The 5233 downstream primer anneals to the 3′ end of the gamma C_{H}1 ORF, and incorporates a termination codon and a Not I site downstream of the terminator for subsequent cloning. PCR reactions were amplified at 97° C. for 1 minute, 25 cycles of 94° C. for 30 seconds, 55° C. for 30 seconds, 72° C. for 1 minute, and 7 minutes at 72° C. The amplification of the desired approximately 700 bp kappa light chains and the approximately 700 bp gamma Fd regions were confirmed by agarose gel electrophoresis.

The KP6 sequence of pLSBC1731 was PCR amplified for Fab cloning. A 100 μL PCR reaction containing 1 μM 5228, 1 μM 5229, 1×Cloned Pfu Buffer, 0.2 mM dATP, 0.2 mM dCTP, 0.2 mM dGTP, 0.2 mM dTTP, 5 Units Cloned Pfu Polymerase and 1 μL pLSBC1731 plasmid. The PCR reaction was amplified at 97° C. for 1 minute, 25 cycles of 94° C. for 30 seconds, 55° C. for 30 seconds, 72° C. for 30 seconds, and 7 minutes at 72° C. The amplification of the desired approximately 120 bp KP6 propeptide encoding sequence was confirmed by agarose gel electrophoresis. To assemble of the Fab preproprotein the KP6 PCR fragment was fused to the heavy chain Fd fragment by sequence overlap extension (SOE). A 70μL PCR reaction containing 0.01 μL pLSBC1731 PCR product from above, 1 μL PCR amplified human VH3 heavy chain Fd (V_{H}-C_{H}1) regions from above, 1×Expand High Fidelity buffer with MgCl₂, 0.29 mM dATP, 0.29 mM dCTP, 0.29 mM dGTP, 0.29 mM dTTP, 2.6 Units Expand High Fidelity enzyme. The PCR reaction was amplified at 97° C. for 30 seconds, 4 cycles of 94° C. for 30 seconds, 50° C. for 1 minute, 72° C. for 1 minute. After 4 cycles, 10 μL of 10 μM 5228 upstream primer, 10 μl of 10 μM 5609 (Seq ID No: 38) downstream primer, 3 μL of 10×Expand buffer and 7 μL of water were added to the PCR reaction which was cycled at 25 cycles of 94° C. for 30 seconds, 72° C. for 1 minute, followed by 5 minutes at 72° C. The amplification of the desired approximately 0.8 Kb KP6 and Fd encoding sequences were confirmed by agarose gel electrophoresis. The 0.8 Kb PCR amplified fragment was electrophoresed on a 1.5% agarose gel with TAE and 0.5 μg/mL ethidium bromide. The fragment was cut from the gel and purified from the agarose slice using QIAquick gel extraction kit following the manufacturers instructions. Briefly, 900 μL of QG buffer was added to the gel fragment, the mixture was incubated at 65° C. for 10 minutes with occasional agitation. The dissolved gel slice was applied to the QIAquick column and centrifuged at 14 K rpm for 1 minute. The column was washed with 750 μL PE and the purified fragment eluted in 50 μL EB. To assemble the Fab, the KP6-heavy chain Fd PCR fragment from above was fused to the 5230-5235 (Seq ID No: 31) primer amplified kappa leader-light chain from above by SOE. A 80 μL PCR reaction containing 1 μL KP6-heavy chain Fd PCR fragment, 1 μL PCR amplified kappa leader-light chain, 1×Expand High Fidelity buffer with MgCl₂, 0.25 mM dATP, 0.25 mM dCTP, 0.25 mM dGTP, 0.25 mM dTTP, 2.6 Units Expand High Fidelity enzyme. The PCR reaction was amplified at 94° C. for 2 minutes, 10 cycles of 94° C. for 30 seconds, 55° C. for 30 seconds, 72° C. for 1 minute and finally 72° C. for 5 minutes. After 10 cycles, 8 μL of 10 μM 5230 upstream primer, 8 μL of 10 μM 5609 downstream primer, and 2 μL of 10×Expand buffer were added to the PCR reaction which was cycled at 94° C. for 5 minutes, 25 cycles of 94° C. for 30 seconds, 55° C. for 30 seconds, 72° C. for 1.5 minutes, followed by 7 minutes at 72° C. The amplification of the desired approximately 1.5 Kb Fab preproprotein encoding sequences were confirmed by agarose gel electrophoresis. The PCR product was purified for subsequent cloning using the QIAquick PCR purification kit per manufacturers instructions. Briefly, the PCR reaction was applied to the QIAquick spin column and centrifuged 14K rpm for 1 minute, washed with 500 μL PB, washed with twice with 750 μL PE and spun dry. The purified PCR product was eluted with 50 μL EB. The purified 1.5 Kb PCR product was subject to restriction endonuclease digestion with Pac I and Not I to produce cohesive ends for cloning. The 200 μL restriction digest contained 50 μL of the above purified PCR product, 100 Units Pac I, 100 Units Not I, 100 μg/mL BSA, 50 mM NaCl, 10 mM Tris-HCl (pH 7.9), 10 mM MgCl₂, 1 mM DTT. The reaction was incubated at 37° C. for 2 hours and subsequently electrophoresed on a 1.5% agarose gel with TAE and 0.5 μg/mL ethidium bromide. The 1.5 Kb Pac I and Not I digested fragment was cut from the gel and purified from the agarose slice using QIAquick gel extraction kit following the manufacturers instructions. Briefly, 600 μL of QG buffer was added to the gel fragment, the mixture was incubated at 65° C. for 10 minutes with occasional agitation. The dissolved gel slice was applied to the QIAquick column and centrifuged at 14K rpm for 1 minute. The column was washed with twice with 750 μL PE, dried and the purified fragment eluted in 50 μL EB. The presence of the approximately 1.5 Kb purified fragment was verified by gel electrophoresis.

The p5PNCAP plasmid was subject to restriction endonuclease digestion with Pac I and Not I to produce cohesive ends for cloning. The 200 μL restriction digest contained 2.5 μg of p5PNCAP plasmid DNA, 50 Units Pac I, 50 Units Not I, 100 μg/mL BSA, 50 mM NaCl, 10 mM Tris-HCl (pH 7.9), 10 mM MgCl₂, 1 mM DTT. The digest was incubated at 37° C. for 3.5 hours, and electrophoresed on a 0.8% agarose gel with TAE and 0.5 μg/mL ethidium bromide to separate the approximately 9.7 Kb fragment from the 0.6 Kb fragment. The 9.7 Kb Pac I and Not I digested fragment was isolated in gel using a scalpel blade. The fragment was purified away from the agarose using QIAquick gel extraction kit following the manufacturers instructions. Briefly, 1.32 mL of QG buffer was added to the gel fragment, the mixture was incubated at 65° C. for 10 minutes with occasional agitation. 10 μL of 3 M NaAcetate and 220 μL of isopropanol was added to one half of the dissolved gel slice which was then applied to the QIAquick column and centrifuged at 14K rpm for 1 minute. The column was washed with 500 μL QB, 750 μL PE and the purified fragment eluted in 50 μL EB. The other half of the dissolved gel slice was processed in the same manner as above and the eluates combined. The presence of the approximately 9.7 Kb purified fragment was verified by gel electrophoresis.

The above prepared 1.5 Kb Pac I and Not I digested Fab preproprotein fragment was cloned into prepared vector p5PNCAP for expression in plants to create clones HuFab (Seq ID No: 87). A 30 μL ligation reaction containing 1 μL Pac I and Not I prepared p5PNCAP, 5 μL Pac I and Not I prepared Fab preproprotein fragment, 800 Units T4 DNA Ligase, 50 mM Tris-HCl (pH 7.5), 10 mM MgCl₂, 25 μg/mL BSA, 10 mM DTT, 1 mM ATP. The reaction was incubated at overnight at 16° C. Bacterial transformation was performed with a Gene Pulser electroporator (BioRad, Hercules, Calif.) following manufacturer recommendations. Briefly, 40 μL of electro-competent JM109 cells were mixed with 2 μL of ligation and transferred to a cold 0.2 cm cuvette. The mixture was pulsed at 2.5 KV, 200 ohms, 25 μFD. After pulsing, 150 μL of SOC was added and the cells allowed to recover for 20 minutes at 37° C. Cells were plated on LB plates containing 100 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.). Briefly, the cells were pelleted by centrifugation at 3K rpm for 15 minutes in a plate centrifuge. The supernatant was drained from the cell pellets and the cells resuspended in 250 μL P1 Buffer by vortexing. 250 μL of P2 was added to the cells, mixed by inverting and incubated for 5 minutes to lyse the cells. 350 μL of N3 was added to the cell lysates, mixed by inverting and transferred to the Turbo Filter plate. A vacuum was applied to the Turbo Filter, which filtered the sample into the QIAprep plate. A vacuum was then applied to the QIAprep plate pulling the sample through the plate and bound the plasmid to the plate membrane. The QIAprep plate was washed using vacuum force with 0.9 mL of PB, followed by two washes with 0.9 mL of PE and vacuum dried. 100 μL EB buffer was added to the purified plasmid, incubated for 1 minute, and subsequently centrifuged for 3 minute at 6K rpm to elute the purified plasmid. Clones were confirmed to contain the 1.5 Kb insert and the 9.7 Kb vector fragments by restriction enzyme mapping with Pac I and Not I followed by agarose gel electrophoresis. The human Fab preproproteins were sequenced using standard methods to verify the proper assembly and identify the variable and constant region sequences.

Infectious transcripts were synthesized in-vitro from each clone using the mMessage mMachine T7 kit (Ambion, Austin, Tex.) following the manufacturers directions. Briefly, a 5.5 μL reaction containing 1 μL 10×Reaction buffer, 2.5 μL 2×NTP/CAP mix, 1 μL Enzyme mix and 3.5 μL plasmid was incubated at 37° C. for 2 hours. The synthesized transcripts were encapsidated in a 40 μL reaction containing 0.1 M Na₂HPO₄—NaH₂PO₄ (pH 7.0), 0.5 mg/mL purified U1 coat protein (LSBC, Vacaville, Calif.) which was incubated overnight at room temperature. 40 μL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate a 20 day post sow Nicotiana benthamiana plant (Dawson, WO et al. (1986) Proc. Natl. Acad Sci. USA 83:1832-1836). High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of Fab protein.

At 12 days post inoculation, systemically infected upper leaves from individual plants were harvested. The secreted protein fraction, or interstitial fluid (IF) was extracted and analyzed for presence of recombinant protein. The leaf tissue was placed in a GF/B 0.8 mL Unifilter (Whatman, Clifton, N.J.), covered with 20 mM Tris-HCl (pH 7.0) and subjected to 760 mmHg vacuum for 30 seconds. The vacuum is released and re-applied three times to completely infiltrate the tissue with buffer. The residual buffer is discarded and the tissue dried by centrifugation at 400 rpm in a plate centrifuge for 10 seconds. The IF fraction is recovered into a 96-well microplate by centrifugation for 10 minutes at 3K rpm in a plate centrifuge. 30 μL of each IF sample was prepared for SDS-PAGE analysis by the addition of 5 μL 5×tris-glycine sample dye containing 10% 2-mercaptoethanol for reducing gels and no 2-mercaptoethanol for non-reducing gels and the mixture was boiled for 2 minutes. Samples were separated on a 15% Criterion gel (Bio-Rad) and the proteins were detected by Coomassie R-250 Brilliant blue staining. Protein banding in the reducing gel at approximately 25 KDa indicates the presence of the desired 25 KDa heavy chain Fd and the 25 KDa light chain. A corresponding protein at approximately 50 KDa under non-reducing conditions as seen in samples HuFab A9, HuFab D5, and HuFab H2 (Seq ID No: 88) are evidence of a assembled, disulfide linked Fab heterodimer consisting of the heavy chain Fd and the kappa light chain. The samples were subjected to western blot analysis to verify the presence of the heavy Fd and light chain polypeptides. The IF samples were diluted 1:10 in 1×tris-glycine sample dye containing 10% 2-mercaptoethanol. 10 μL of each sample was loaded on two separate Novex 10-20% tris glycine gels and subsequently transferred to Nitrocellulose membrane using the Xcell II Blot (Invitrogen, Carlsbad, Calif.) following manufacturers instructions. The membranes were blocked overnight in TBST containing 2.5% powdered skim milk and 2.5% BSA. One membrane was probed with a 1:4000 dilution of Goat anti-human kappa-HRP labeled sera and the second membrane was probed with 1:4000 dilution of Goat anti-human IgG-HRP labeled sera (Southern Biotechnology, Birmingham, Ala.) for 1 hour at room temperature. The blots were washed three times in TBST and the labeled proteins detected with the ECL+plus Western Blotting Detection System (Amersham Biosciences, Buckinghamshire, England). The anti kappa sera detected an approximately 25 KDa proteins in the HuFab A9, HuFab D5 and HuFab H2 samples and a corresponding approximately 25 KDa protein was detected with the anti gamma sera indicating that both the heavy Fd and kappa chains were expressed and secreted.

### EXAMPLE 3

Cloning of the 9E10 Heavy Chain and Light Chain Genes

Mouse hybridoma line Myc 1-9E10.2 expresses a murine monoclonal antibody (IgG1) that recognizes a human c-myc epitope of amino acid sequence EQKLISEEDL (G. I Evans et al., Molec. Cell. Biol. 5: 3610-3616, 1985). Cells were obtained from ATCC (CRL-1729) and cultured under standard conditions. 2×10⁶ cultured cells were spun and washed to remove excess culture media and lysed with 600 μL RLT buffer containing 1% 2-mercaptoethanol (Qiagen, Valencia, Calif.). Total RNA was purified using the QIAshredder and RNEASY column per manufacturers directions. Briefly, the cell lysate was applied to the QIAshredder column and spun in a centrifuge for 2 minutes at 14K rpm. The flow through was collected and diluted with an equal volume of 70% ethanol. The mixture was transferred to a RNeasy column and centrifuged for 15 seconds at 10K rpm until all sample was processed through the column. The RNA bound to the column was washed with 700 μL RW1 followed by a wash with 500 μL RPE and subsequently dried. The purified RNA was eluted in 50 μL RNASE free water by centrifugation for 1 minute at 10K rpm. First strand cDNA was synthesized from 0.8 μg total RNA using a SMART 3′ RACE kit (BD Biosciences Clontech, Palo Alto, Calif.) with 1 μL 3′ CDS primer in 5 μL. The RNA primer mix was heated to 70° C. for 2 minutes and placed on ice for an additional 2 minutes. To the RNA and primer mix was brought to 10 μL containing 1×First strand buffer, 2 mM DTT,1 mM dATP, 1 mM dCTP, 1 mM dGTP, 1 mM dTTP and 1 μL Powerscript Reverse Transcriptase. The reaction was incubated at 42° C. for 90 minutes and then 100 μL of Tricine-EDTA Buffer (10 mM Tricine-KOH, pH 8.5, 1 mM EDTA) was added and the reaction heated to 72° C. for 7 minutes. The 9E10 kappa light chain was PCR amplified in a 50 L reaction containing 1×Advantage 2 PCR Buffer, 0.2 mM dATP, 0.2 mM dCTP, 0.2 mM dGTP, 0.2 mM dTTP, 1×Advantage 2 Polymerase Mix, 2.5 μL of prepared cDNA, 1×UPM, and 0.2 μM 9E10k15′ (Seq ID No: 50). The 9E10k15′ primer was designed to anneal to the murine kappa light leader sequence from germline sequence V-21C9.5KB′CL. The reaction was cycled 5 times at 94° C. for 5 seconds, 72° C. for 3 minutes followed by 5 times at 94° C. for 5 seconds, 70° C. for 10 seconds, 72° C. for 3 minutes and 25 cycles at 94° C. for 5 seconds, 67° C. for 10 seconds, 72° C. for 3 minutes. The amplification of the desired approximately 900 bp fragment was confirmed by agarose gel electrophoresis. The 9E10 heavy chain was PCR amplified in a 50 L reaction containing 1×Advantage 2 PCR Buffer, 0.2 mM dATP, 0.2 mM dCTP, 0.2 mM dGTP, 0.2 mM dTTP, 1×Advantage 2 Polymerase Mix, 2.5 μL of prepared cDNA, 1×UPM, and 0.4 μM 9E10gfw5′ (Seq ID No: 51). The 9E10gfw5′ primer was designed to anneal to the murine heavy chain variable FR1 sequence identified from germline sequence Vh7183(Vh69.1). The reaction was cycled 5 times at 94° C. for 5 seconds, 70° C. for 3 minutes followed by 5 times at 94° C. for 5 seconds, 68° C. for 10 seconds, 72° C. for 3 minutes and 25 cycles at 94° C. for 5 seconds, 64° C. for 10 seconds, 72° C. for 3 minutes. The amplification of the desired approximately 1.6 Kb fragment was confirmed by agarose gel electrophoresis.

The prepared PCR fragments from above were cloned into pCR4-TOPO (Invitrogen) following the manufacturers directions to create plasmid p9E10Hy-TOPO (Seq ID No: 77) and p9E10Lt-TOPO (Seq ID No: 79). Briefly, 2 μL of PCR product, 1 μL vector, 1 μL of salt solution and 1 μL of water were mixed, incubated at room temperature for 5 minutes. The ligations were placed on ice and 25 μL of chemically competent Top 10 cells was added to each ligation and the mixes were incubated on ice for 10 minutes. The transformation reactions were heat shocked by incubating at 42° C. for 30 seconds and immediately placed on ice and 250 μL of SOC was added. The transformations were allowed to recover by incubating at 37° C., 200 rpm shaking for 20 minutes. The transformations were plated out on LB plates containing ampicillin and grown overnight at 37° C. Individual colonies were used to inoculate 1.0 mL Super Broth (SB) containing 100 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.). Briefly, the cells were pelleted by centrifugation at 3 K rpm for 15 minutes in a plate centrifuge. The supernatant was drained from the cell pellets and the cells resuspended in 250 μL P1 Buffer by vortexing. 250 μL of P2 was added to the cells, mixed by inverting and incubated for 5 minutes to lyse the cells. 350 μL of N3 was added to the cell lysates, mixed by inverting and transferred to the Turbo Filter plate. A vacuum was applied to the Turbo Filter, which filtered the sample into the QIAprep plate. A vacuum was then applied to the QIAprep plate pulling the sample through the plate and bound the plasmid to the plate membrane. The QIAprep plate was washed using vacuum force with 0.9 mL of PB, followed by two washes with 0.9 mL of PE and vacuum dried. 100 μL EB buffer was added to the purified plasmid, incubated for 1 minute, and subsequently centrifuged for 3 minute at 6K rpm to elute the purified plasmid. The presence of the approximately 1.2 Kb insert for p9E10Hy-TOPO (Seq ID No: 77) and 700 bp for p9E10Lt-Topo (Seq ID No: 79) was verified with EcoRI restriction digest and agarose gel electrophoresis. The purified p9E10Hy-TOPO and p9E10Lt-TOPO plasmids were subjected to nucleic acid sequencing using standard methods to verify the 9E10 heavy chain and kappa chain sequences.

### EXAMPLE 4

Cloning of the 9E10 Fab Proprotein and expression analysis

The KP6 sequence of pLSBC1731 was PCR amplified for Fab cloning. A 100 μL PCR reaction containing 1 μM 5228, 1 μM 5229, 1×Expand High Fidelity Buffer, 0.2 mM dATP, 0.2 mM dCTP, 0.2 mM dGTP, 0.2 mM dTTP, 3.5 Units Expand High Fidelity and 1 μL pLSBC1731 plasmid. The PCR reaction was amplified at 97° C. for 1 minute, 25 cycles of 94° C. for 30 seconds, 55° C. for 30 seconds, 72° C. for 30 seconds, and 7 minutes at 72° C. The amplification of the desired approximately 120 bp KP6 propeptide encoding sequence was confirmed by agarose gel electrophoresis. The 9E10 kappa light chain was amplified with primers 6056 (Seq ID No: 41) and 2228 from p9E10Lt-TOPO and the 9E10 heavy Chain Fd (V_{H}C_{H}1) was amplified with 2225 (Seq ID No: 39) and 6055 (Seq ID No: 40) from p9E10Hy-TOPO for Fab proprotein cloning. Each primer set incorporated additional regions encoding the termini of the KP6 propeptide coding sequence pLSBC1731 at either the 5′ or 3′ end, as well as a restriction site for cloning into the appropriate expression vector. (either Sph1 at the 5′ end of the heavy chain fragment or AvrII at the 3′ end of the light chain fragment). A 100 μL PCR reaction containing 1 μM upstream, 1 μM downstream, 1×Expand High Fidelity Buffer with MgCl₂, 0.2 mM dATP, 0.2 mM dCTP, 0.2 mM dGTP, 0.2 mM dTTP, 3.5 Units Expand High Fidelity and 1 μL plasmid. The PCR reaction was amplified at 97° C. for 1 minute, 25 cycles of 94° C. for 30 seconds, 55° C. for 30 seconds, 72° C. for 30 seconds, and 7 minutes at 72° C. The amplification of the desired approximately 700 bp kappa chain encoding sequence was confirmed by agarose gel electrophoresis. The light chain fragment was fused to the KP6 PCR fragment by sequence overlap extension (SOE). A 80 μL PCR reaction containing 0.5 μL pLSBC1731 PCR product from above, 0.5 μL PCR amplified 9E10 kappa light chain (V_{L}C_{L}) regions from above, 1×Expand High Fidelity buffer with MgCl₂, 0.2 mM dATP, 0.2 mM dCTP, 0.2 mM dGTP, 0.2 mM dTTP, 3.5 Units Expand High Fidelity enzyme. The PCR reaction was amplified at 94° C. for 1 minute, 25 cycles of 94° C. for 30 seconds, 55° C. for 30 seconds, 72° C. for 1 minute and 5 minutes at 72° C. After the 25 cycles, 9 μL of 10 μM 5228 upstream primer, 9 μl of 10 μM 2228 downstream primer, were added to the PCR reaction which was cycled at 25 cycles of 94° C. for 30 seconds, 55° C. for 30 seconds, 72° C. for 1 minute and 5 minutes at 72° C. The amplification of the desired approximately 0.8 Kb KP6 and light chain encoding sequences were confirmed by agarose gel electrophoresis. To assemble of the 9E10 Fab proprotein, the KP6-light chain PCR fragment from above was fused to the amplified 9E10 heavy chain Fd from above by SOE. A 80 μL PCR reaction containing 0.5 μL KP6-light chain PCR fragment, 0.5 μL PCR amplified heavy chain Fd, 1×Expand High Fidelity buffer with MgCl₂, 0.2 mM dATP, 0.2 mM dCTP, 0.2 mM dGTP, 0.2 mM dTTP, 3.5 Units Expand High Fidelity enzyme. The PCR reaction was amplified at 97° C. for 2 minutes, 10 cycles of 94° C. for 30 seconds, 55° C. for 30 seconds, 72° C. for 1 minute and finally 72° C. for 5 minutes. After 10 cycles, 9 μL of 10 μM 2225 upstream primer, 9 μL of 10 ,M 2228 downstream primer were added to the PCR reaction which was cycled at 97° C. for 2 minutes, 10 cycles of 94° C. for 30 seconds, 55° C. for 30 seconds, 72° C. for 1.5 minutes, followed by 5 minutes at 72° C. The amplification of the desired approximately 1.5 Kb 9E10 Fab proprotein encoding sequences were confirmed by agarose gel electrophoresis. The PCR product was purified for subsequent cloning using the QIAquick PCR purification kit per manufacturers instructions. Briefly, the PCR reaction was applied to the QIAquick spin column and centrifuged 14K rpm for 1 minute, washed with 500 μL PB, washed with twice with 750 μL PE and spun dry. The purified PCR product was eluted with 50 μL EB. The purified 1.5 Kb PCR product was subject to restriction endonuclease digestion with Sph I and Avr II to produce cohesive ends for cloning. The 50 μL restriction digest contained 25 μL of the above purified PCR product, 8 Units Sph I, 8 Units Avr II, 100 μg/mL BSA, 50 mM NaCl, 10 mM Tris-HCl (pH 7.9), 10 mM MgCl₂, 1 mM DTT. The reaction was incubated at 37° C. for 2 hours and subsequently electrophoresed on a 1% agarose gel with TAE and 0.5 μg/mL ethidium bromide. The 1.5 Kb Sph I and Avr II digested fragment was cut from the gel and purified from the agarose slice using QIAquick gel extraction kit following the manufacturers instructions. Briefly, 600 μL of QG buffer was added to the gel fragment, the mixture was incubated at 65° C. for 10 minutes with occasional agitation. The dissolved gel slice was applied to the QIAquick column and centrifuged at 14K rpm for 1 minute. The column was washed with twice with 750 μL PE, dried and the purified fragment eluted in 50 μL EB. The presence of the approximately 1.5 Kb purified fragment was verified by gel electrophoresis.

The 1.5 Kb Sph I and Avr II 9E10 Fab proprotein was cloned into the SphI and Avr II prepared p1324-MBP plasmid to create pLSBC1736 (Seq ID No: 85). A 50 μL ligation reaction containing 10 μL prepared 9E10 Fab proprotein, 0.4 μg p1324-MBP, 1200 Units T4 DNA Ligase, 50 mM Tris-HCl (pH 7.5), 10 mM MgCl₂, 25 μg/mL BSA, 10 mM DTT, 1 mM. ATP was incubated at 14° C. overnight. The ligation was precipitated with 3 volumes ethanol and 0.3 volumes 10 M NH₄Acetate, spun and washed with 70% ethanol. The pellets were resuspended in 20 μL 10 mM Tris-HCL (pH 8.0). Bacterial transformations were performed with a Gene Pulser electroporator (BioRad, Hercules, Calif.) following manufacturer recommendations. Briefly, 40 μL of electro-competent JM109 cells were mixed with 4 μL of ligation and transferred to a cold 0.2 cm cuvette. The mixture was pulsed at 2.5 KV, 200 ohms, 25 μFD. After pulsing, 120 μL of SOC was added and the cells allowed to recover for 20 minutes at 37° C. Cells were plated on LB plates containing 100 μg/mL ampicillin and grown overnight at 37° C. Individual colonies were picked and used to inoculate 1 mL Super Broth (SB) containing 400 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif. as previously described and eluted in 100 μL EB Buffer. pLSBC1736 (Seq ID No: 85) clones were confirmed to contain the 1.5 Kb insert and the 9.7 Kb vector fragments by restriction enzyme mapping with Sph I and Avr II followed by agarose gel electrophoresis. The 9E10 Fab proprotein was sequenced using standard methods to verify the sequence.

Infectious transcripts were synthesized in-vitro from the pLSBC1736 clone using the mMessage mMachine T7 kit (Ambion, Austin, Tex. following the manufacturers directions. Briefly, a 5.5 μL reaction containing 1 μL 10×Reaction buffer, 2.5 μL 2×NTP/CAP mix, 1 μL Enzyme mix and 3.5 μL plasmid was incubated at 37° C. for 2 hours. The synthesized transcripts were encapsidated in a 40 μL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 20 μL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate a 19 day post sow Nicotiana benthamiana plant (Dawson, WO et al. (1986) Proc. Natl. Acad Sci. USA 83:1832-1836). High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of Fab protein.

Interstitial fluid from infected leaves of each plant was harvested 8 days post inoculation and screened by ELISA. Systemically infected upper leaves from individual plants were harvested. The secreted protein fraction, or interstitial fluid (IF) was extracted and analyzed for presence of recombinant protein. The leaf tissue was placed in a GF/B 0.8 mL Unifilter (Whatman, Clifton, N.J.), covered with 20 mM Tris-HCl (pH 7.0) and subjected to 760 mmHg vacuum for 30 seconds. The vacuum is released and re-applied three times to completely infiltrate the tissue with buffer. The residual buffer is discarded and the tissue dried by centrifugation at 400 rpm in a plate centrifuge for 30 seconds. The IF fraction is recovered into a 96-well microplate by centrifugation for 10 minutes at 3K rpm in a plate centrifuge.

20 μL of each IF sample was prepared for SDS-PAGE analysis by the addition of 5 μL 5×tris-glycine sample dye containing 10% 2-mercaptoethanol for reducing gels and no 2-mercaptoethanol for non-reducing gels and the mixture was boiled for 2 minutes. Samples were separated on a 10-20% gradient Criterion gel (Bio-Rad) and the proteins were detected by Coomassie R-250 Brilliant blue staining. Protein banding in the reducing gel at approximately 25 KDa indicates the presence of the desired 25 KDa heavy chain Fd and the 25 KDa light chain. A corresponding protein at approximately 50 KDa under non-reducing conditions was seen as evidence of an assembled, disulfide linked Fab heterodimer consisting of the heavy chain Fd and the kappa light chain.

To perform western analysis, 20 μL of reduced and nonreduced sample were loaded on 10-20% Criterion Tris glycine gel and transferred to Nitrocellulose membrane. The membranes were blocked overnight in TBST containing 2.5% powdered skim milk and 2.5% BSA. One membrane was probed with a 1:3000 dilution of Goat anti-mouse kappa-HRP labeled sera and the second membrane was probed with 1:3000 dilution of Goat anti-mouse IgG-HRP labeled sera (Southern Biotechnology, Birmingham, Ala.) for 1 hour at room temperature. The blots were washed three times in TBST and the labeled proteins detected with the ECL+plus Western Blotting Detection System (Amersham Biosciences, Buckinghamshire, England). The anti kappa sera detected an approximately 25 KDa proteins on the reduced sample and an approximately 50 KD band on the non-reduced indicating the presence of interchain disulfide bridges and an assembled 9E10 Fab. The anti gamma sera detected an approximately 25 KDa proteins on the reduced sample and a approximately 50 KD band on the non-reduced indicating the presence of interchain disulfide bridges and an assembled 9E10 Fab.

The ability of the recombinant 9E10 Fab protein from pLSBC1736 to recognize the antigen c-myc was verified by Western analysis where myc-tagged GFP (Invitrogen, Carlsbad, Calif.) was transferred to nitrocellulose paper and probed with crude IF material purified from plants infected with pLSBC1736. Samples containing 250 ng of myc-tagged GFP, or 30 ng of GFP in 1×SDS/PAGE buffer were boiled and run on a 10-20% Criterion Tris glycine gel and transferred to Nitrocellulose membrane. The membrane was blocked overnight in TBST containing 2.5% powdered skim milk and 2.5% BSA. The membrane was probed with a 1:3000 dilution of Goat anti-mouse kappa-HRP labeled sera (Southern Biotechnology, Birmingham, Ala.) for 1 hour at room temperature. The blots were washed three times in TBST and the labelled proteins detected with the ECL+plus Western Blotting Detection System (Amersham Biosciences, Buckinghamshire, England). A band was visualized in the lane containing myc tagged GFP corresponding to the expected size of 53 KDa, and there were no detected proteins in the untagged GFP control lane. There were no bands visualized in lanes which were probed with IF obtained from healthy plants. The specific recognition of the myc-tagged GFP protein with IF from pLSBC1736 infected Nicotiana banthamiana plants containing the 9E10 Fab demonstrates the proper activity of the disulfide linked heteromultimeric protein.

### EXAMPLE 5

Cloning and Expression of 9E10 MAB

A 9E10 monoclonal antibody artificial proprotein was assembled by fusing the 9E10 kappa light chain to the KP6 propeptide region of pLSBC1731, which was fused to the 9E10 gamma heavy chain. This fusion will result in a first domain light chain, the second domain propeptide and the third domain the complete heavy chain sequence. The 9E10 kappa light chain was PCR amplified from plasmid p9E10Lt-TOPO with upstream primer 2230 (Seq ID No: 4) and downstream primer 6057. The 9E10 gamma heavy chain was PCR amplified from plasmid p9E10Hy-TOPO and with upstream primer 6058 (Seq ID No: 14) and downstream primer 2227. Separate 100 μL PCR reactions containing 1 μM upstream, 1 μM downstream, 1×Expand High Fidelity Buffer with MgCl₂, 0.2 mM dATP, 0.2 mM dCTP, 0.2 mM dGTP, 0.2 mM dTTP, 3.5 Units Expand High Fidelity and 1μL plasmid template were amplified at 94° C. for 1 minute, 30 cycles of 94° C. for 30 seconds, 55° C. for 30 seconds, 72° C. for 1 minute, and a final step of 7 minutes at 72° C.

The amplification of the desired approximately 120 bp KP6 propeptide encoding sequence, 700 bp 9E10 kappa light chain encoding sequence and 1.3 Kb 9E10 gamma heavy chain encoding sequence were confirmed by agarose gel electrophoresis. To assemble of the 9E10 MAb proprotein, the amplified 9E10 kappa light chain, the pLSBC1731 KP6 PCR fragment, and the amplified 9E10 gamma heavy chain were fused by sequence overlap extension (SOE).

A 25 μL PCR reaction containing 0.1 μL pLSBC1731 PCR fragment, 0.1 μL PCR amplified 9E10 gamma heavy chain, 0.1 μL PCR amplified 9E10 kappa light chain, 1×Expand High Fidelity buffer with MgCl₂, 0.2 mM dATP, 0.2 mM dCTP, 0.2 mM dGTP, 0.2 mM dTTP, 3.5 Units Expand High Fidelity enzyme was amplified at 97° C. for 1 minute, 15 cycles of 94° C. for 30 seconds, 55° C. for 2 minutes, 72° C. for 90 seconds and a final step of 72° C. for 5 minutes. The PCR reaction was purified using the MinElute PCR purification kit (Qiagen) following the manufacturers instructions. Briefly, 5 volumes of PB buffer was added to the reaction, mixed, applied to the column and centrifuged at 14K rpm for 1 minute. The column was washed with 750 μL Buffer PE and the purified fragment eluted in 10 μL EB. A 50 μL reaction containing 5 μL purified PCR product, 50 mM potassium acetate, 20 mM Tris-Acetate pH 7.9, 1 mM DTT, 10 mM magnesium acetate, 20 Units SphI and 8 Units Avr II was incubated at 37° C. for 1 hour and electrophoresed on a 1.0% agarose gel with TAE and 0.5 μg/mL ethidium bromide to separate the approximately 2.3 Kb 9E10 MAb proprotein encoding sequence. The 2.3 Kb MAb proprotein encoding sequence was purified using the QIAquick gel extraction kit (Qiagen) following manufacturers recommendations and eluted with 50 μL EB Buffer.

The 2.3 Kb SphI and Avr II digested fragment of 9E10 MAb proprotein encoding fragment was ligated into the SphI and Avr II prepared pLSBC1324 plasmid to create pLSBC1799 (Seq ID No: 115). A 30 μL ligation reaction containing 23 ,μL prepared SphI and Avr II 9E10 MAb prepared PCR fragment, 0.4 μg SphI and Avr II pLSBC1324 fragment, 1200 Units T4 DNA Ligase, 50 mM Tris-HCl (pH 7.5), 10 mM MgCl₂, 25 μg/mL BSA, 10 mM, DTT, 1 mM ATP was incubated at 14° C. overnight. The ligation reaction was ethanol precipitated and the pellet was resuspended in 10 μL water and 2 μL used to transform electrocompetent JM109 as previously described. Cells were plated on LB plates containing 50 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and plasmid eluted with 100 μL EB buffer. Clones were confirmed to contain the 2.3 Kb insert and the 9.7 Kb vector fragments by restriction enzyme mapping with SphI and Avr II followed by agarose gel electrophoresis. The 9E10 MAb proprotein was sequenced using standard methods to verify the sequence.

### EXAMPLE 6

Cloning of the S1C5 Heavy Chain and Light Chain Genes

The murine monoclonal antibody S1C5 recognizes the idiotope of the surface immunoglobulin of the murine B cell tumor 38C13 (Maloney et al., Hybridoma. 4:191-209, 1985). Cells were cultured under standard techniques. The heavy chain and kappa light chain genes were isolated by PCR amplification of cDNA produced from hybridoma mRNA. Briefly, 1×10⁶ cultured cells were spun and washed to remove excess culture media and lysed with 600 μL RLT buffer containing 1% 2-mercaptoethanol (Qiagen, Valencia, Calif.). Total RNA was purified using the QIAshredder and RNEASY column per manufacturers directions. Briefly, the cell lysate was applied to the QIAshredder column and spun in a centrifuge for 2 minutes at 15K rpm. The flow through was collected and diluted with an equal volume of 70% ethanol. The mixture was transferred to a RNeasy column and centrifuged for 15 seconds at 10K rpm until all sample was processed through the column. The RNA bound to the column was washed with 700 μL RW1 followed by a wash with 500 μL RPE and subsequently dried. The purified RNA was eluted in 50 μL RNASE free water by centrifugation for 1 minute at 10K rpm. 5 μg of the above prepared total RNA was incubated at 70° C. for 2 minutes and then applied to 20 μL of magnetic beads in binding buffer (20 mM Tris-HCl (pH 7.5), 1.0 M LiCl, 2 mM EDTA) where the beads were prepared by washing with 50 μL binding buffer. The RNA and bead mixture were incubated at room temperature for 5 minutes with constant rotating. The supernatant containing unbound material was removed and the beads were washed with 100 μL washing buffer (10 mM Tris-HCl (pH 7.5), 0.15 M LiCl, 1 mM EDTA) followed by the addition of 40 μL nuclease free water. Complementary DNA (cDNA) was synthesized in 100 μL reactions containing 60 mM Tris HCl (pH 8.3), 90 mM KCl, 4 mM MgCl₂, 12 mM DTT, 240 Units RNasin (Promega, Madison, Wis.), 2400 Units Superscript II (Invitrogen, Carlsbad, Calif.), 0.6 mM dATP, 0.6 mM dCTP, 0.6 mM dGTP, 0.6 mM dTTP, and the oligo dT bound RNA from above. The reaction was incubated at 42° C. for 90 minutes with constant rotation. The supernatant was removed from the magnetic beads. The beads were then washed with 50 ul 50 mM potassium acetate, 20 mM Tris-Acetate pH 7.9, 1 mM DTT, 10 mM magnesium acetate and resuspended in 220 μL 50 mM potassium acetate, 20 mM Tris-Acetate pH 7.9, 1 mM DTT, 10 mM magnesium acetate, 2.5 mM CoCl₂, 0.2 mM dGTP and 44 Units terminal transferase (New England BioLabs). The reaction mixture was incubated for 40 minutes at 37 ° C.

The S1C5 kappa light chain was PCR amplified with upstream primer C-anchor (Seq ID No: 1), which anneals to the poly-G leader and downstream primer 2228 (Seq ID No: 5), which anneals to the 3′ end of the kappa light constant chain and incorporates an Avr II site downstream of the termination codon for subsequent cloning. A 100 μL PCR reactions containing 1 μM upstream, 1 μM downstream, 1×Taq Polymerase Buffer, 0.2 mM dATP, 0.2 mM dCTP, 0.2 mM dGTP, 0.2 mM dTrP, 25 Units Taq DNA Polymerase (Stratagene) and 5 μL prepared cDNA. The PCR reactions were amplified at 97° C. for 1 minute, 30 cycles of 94° C. for 30 seconds, 50° C. for 1 minute, 72° C. for 1 minute, and a final 7 minute incubation at 72° C. The PCR amplified product were electrophoresed on a 1% agarose gel with TAE and 0.5 μg/mL ethidium bromide. The 0.7 Kb band was excised and purified using the QIAquick gel extraction kit as previously described and eluted with 50 μL elution buffer. The amplified S1C5 kappa light chain fragment was cloned into pCR4-TOPO (Invitrogen) following the manufacturers directions to create plasmid pLSBC1757. Briefly, 1 μL of PCR product, 1 μL vector, 1 μL of salt solution and 3 μL of water were mixed, incubated at room temperature for 5 minutes. The ligation was placed on ice and 25 μL of chemically competent Top 10 cells was added to the ligation and the mix was incubated on ice for 10 minutes. The transformation reaction was heat shocked by incubating at 42° C. for 30 seconds and immediately placed on ice and 250 μL of SOC was added. The transformation was allowed to recover by incubating at 37° C., 200 rpm shaking for 20 minutes. The transformation was plated out on LB plates containing ampicillin and grown overnight at 37° C. Individual colonies were used to inoculate 1.0 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN) as previously described. Clones were digested with EcoR1 and screened for the presence of a 0.7 Kb insert band. The purified pLSBC1757 plasmid was subjected to nucleic acid sequencing using standard methods.

The S1C5 heavy chain was PCR amplified with degenerate upstream primers 5′ MH1 and 5′ MH2 described by Wang et. al., J. of Imm. Methods, 233: 167-177 (2000), and downstream primer 2227 (Seq ID No: 3), which anneals to the 3′ end of the gamma constant chain and incorporates an Avr II site downstream of the termination codon for subsequent cloning. A 100 μL PCR reaction containing 0.5 μM 5′ MH1, 0.5 μM 5′ MH2, 1 μM 2227, 1×Taq DNA Polymerase Buffer, 0.2 mM dATP, 0.2 mM dCTP, 0.2 mM dGTP, 0.2 mM dTTP, 25 Units Taq DNA Polymerase (Stratagene) and 5 μL prepared cDNA. The PCR reactions were amplified at 94° C. for 3 minutes, 30 cycles of 94° C. for 1 minute, 45° C. for 1 minute, 72° C. for 2 minutes, and a final 10 minute incubation at 72° C. The PCR amplified product was electrophoresed on a 1% agarose gel with TAE and 0.5 μg/mL ethidium bromide. The 1.3 Kb band was excised and purified using the QIAquick gel extraction kit as previously described and eluted with 50 μL elution buffer. The amplified S1C5 heavy chain fragment was cloned into pCR2.1-TOPO (Invitrogen) following the manufacturers directions to create plasmid pLSBC2523 (Seq ID No: 117). Briefly, 1 μL of PCR product, 1 μL vector, 1 μL of salt solution and 3 μL of water were mixed, incubated at room temperature for 5 minutes. The ligation was placed on ice and 25 μL of chemically competent Top 10 cells was added to the ligation and the mix was incubated on ice for 10 minutes. The transformation reaction was heat shocked by incubating at 42° C. for 30 seconds and immediately placed on ice and 250 μL of SOC was added. The transformation was allowed to recover by incubating at 37° C., 200 rpm shaking for 20 minutes. The transformation was plated out on LB plates containing ampicillin and grown overnight at 37° C. Individual colonies were used to inoculate 1.0 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN) as previously described. Clones were digested with EcoR1 and screened for the presence of a 1.3 Kb insert band. The purified pLSBC2523 plasmid was subjected to nucleic acid sequencing using standard methods.

Construction pLSBC1786

The KP6 sequence of pLSBC1731 was PCR amplified for Fab cloning. A 100 μL PCR reaction containing 1 μM 5228, 1 μM 5229, 1×Expand High Fidelity Buffer, 0.2 mM dATP, 0.2 mM dCTP, 0.2 mM dGTP, 0.2 mM dTTP, 3.5 Units Expand High Fidelity and 1 μL pLSBC1731 plasmid. The PCR reaction was amplified at 97° C. for 1 minute, 25 cycles of 94° C. for 30 seconds, 55° C. for 30 seconds, 72° C. for 30 seconds, and 7 minutes at 72° C. The amplification of the desired approximately 120 bp KP6 propeptide encoding sequence was confirmed by agarose gel electrophoresis.

The S1C5 kappa light chain was amplified from plasmid pLSBC1757 (Seq ID No: 119). The 7659 (Seq ID No: 7) upstream primer anneals to the FR1 region of the S1C5 V_{L} and contains a Ngo MIV site compatible for cloning into vector pLSBC1767, and 6057 (Seq ID No: 6) downstream primer anneals to the 3′ end of the kappa C_{L} ORF, removes the termination codon and fuses the kappa C_{L} ORF in frame to the 5′ end of the KP6 propeptide coding sequence. The S1C5 heavy chain Fd (V_{H}C_{H}1) region was amplified with primers 7660 (Seq ID No: 8) and 7662 (Seq ID No: 9) from plasmid pLSBC2523 for Fab proprotein cloning. The 7660 upstream primer anneals to the 5′ end of the S1C5 V_{H} region and fuses it in frame to the 3′ end of the KP6 propeptide coding sequence and the 7662 downstream primer anneals to the 3′ end of the C_{H}1 domain including a translational termination codon followed by an Avr II site for subsequent cloning. Separate 100 μL PCR reactions containing 1 μM upstream primer, 1 μM downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity and 1 μL plasmid template. The PCR reaction was amplified at 94° C. for 1 minute, 30 cycles of 94° C. for 30 seconds, 55° C. for 30 seconds, 72° C. for 1 minute, and a final step of 7 minutes at 72° C. The amplification of the desired approximately 700 bp S1C5 kappa light chain and 700 bp S1C5 Fd region was confirmed by agarose gel electrophoresis. To assemble of the S1C5 Fab proprotein, the amplified S1C5 kappa light chain, the pLSBC1731 KP6 PCR fragment, and the amplified S1C5 Fd fragment were fused by sequence overlap extension (SOE). A 25 μL PCR reaction containing 0.1 μL pLSBC1731 PCR product from above, 0.1 μL PCR amplified S1C5 Fd (V_{H}C_{Hl}) region, 0.1 μL PCR amplified S1C5 kappa light region, 1×Expand High Fidelity buffer with MgCl₂, 0.2 mM dATP, 0.2 mM dCTP, 0.2 mM dGTP, 0.2 mM dTTP, 3.5 Units Expand High Fidelity enzyme. The PCR reaction was amplified at 97° C. for 1 minute, 15 cycles of 94° C. for 30 seconds, 55° C. for 2 minutes, 72° C. for 90 seconds, and a final step of 72° C. for 5 minutes. The PCR amplified product was electrophoresed on a 1% agarose gel with TAE and 0.5 82 g/mL ethidium bromide. The 1.4 Kb band was excised and purified using the QIAquick gel extraction kit as previously described and eluted with 50 μL elution buffer. The amplified S1C5 Fab proprotein encoding sequence was cloned into pCR4-TOPO (Invitrogen) following the manufacturers directions to create plasmid pLSBC1786. Briefly, 4 μL of PCR product, 1 μL vector, 1 μL of salt solution and 2 μL of water were mixed, and incubated at room temperature for 5 minutes. The ligation was used to transform chemically competent Top 10 cells as described previously described. The transformation was plated out on LB plates containing ampicillin and grown overnight at 37° C. Individual colonies were used to inoculate 1.0 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN) as previously described. Clones were digested with Avr II and Ngo MIV and screened for the presence of a 1.4 Kb insert band. The purified pLSBC1786 plasmid was subjected to nucleic acid sequencing using standard methods.

Construction of pLSBC1792 (Seq ID No: 121)

Plasmid pLSBC1786 was subjected to restriction endonuclease digestion with NgoMIV. A 50 μL reaction containing 5 μL plasmid DNA, 50 mM potassium acetate, 20 mM Tris-Acetate pH 7.9, 1 mM DTT, 10 mM magnesium acetate, 20 Units NgoMIV was incubated at 37° C. for 2.5 hours and electrophoresed on a 1.0% agarose gel with TAE and 0.5 μg/mL ethidium bromide to separate the approximately 3.6 Kb S1C5 Fab proprotein encoding sequence. The 3.6 Kb Fab proprotein encoding sequence was purified using the QIAquick gel extraction kit (Qiagen) following manufacturers recommendations and eluted with 50 μL EB Buffer. The purified fragment was subjected to restriction endonuclease digestion with Avr II. A 60 μL reaction containing 50 μL purified fragment, 50 mM NaCl, 100 mM Tris-HCl pH 7.9, 10 mM MgCl₂, 1 mM DTT, 12 Units Avr II was incubated at 37° C. for 35 minutes and electrophoresed on a 1.0% agarose gel with TAE and 0.5 μg/mL ethidium bromide to separate the approximately 1.5 Kb S1C5 Fab proprotein encoding sequence. The 1.5 Kb Fab proprotein encoding sequence was purified using the QIAquick gel extraction kit (Qiagen) following manufacturers recommendations and eluted with 50 μL EB Buffer. The presence of the approximately 1.5 Kb NgoMIV and Avr II purified fragment of pLSBC1786 was verified by gel electrophoresis.

The 1.5 Kb NgoM VI and Avr II digested fragment of pLSBC1786 was ligated into the NgoMIV and Avr II prepared pLSBC1767 plasmid to create pLSBC1792 (Seq ID No: 121). A 50 μL ligation reaction containing 10 μL prepared NgoM VI and Avr II pLSBC1786 fragment, 0.4 μg NgoM VI and Avr II pLSBC1767 fragment, 1200 Units T4 DNA Ligase, 50 mM Tris-HCl (pH 7.5), 10 mM MgCl₂, 25 μg/mL BSA, 10 mM DTT, 1 mM ATP was incubated at 14° C. overnight. Bacterial transformations with DH5α competent cells (Invitrogen) were performed according to manufacturer recommendations. Cells were plated on LB plates containing 100 μg/mL ampicillin and grown overnight at 37° C. Individual colonies were picked and used to inoculate 1 mL Super Broth (SB) containing 400 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and eluted in 100 μL EB Buffer. pLSBC1792 clones were confirmed to contain a 2.7 Kb fragment by restriction enzyme mapping with Kpn I. The S1C5 Fab proprotein was sequenced using standard methods to verify the sequence.

Construction of pLSBC1798

A S1C5 monoclonal antibody artificial proprotein was assembled by fusing the pLSBC1757 S1C5 kappa light chain to the KP6 propeptide region of pLSBC1731, which was fused to the S1C5 gamma heavy chain of pLSBC2523. This fusion will result in a first domain light chain, the second domain propeptide and the third domain the complete heavy chain sequence to create pLSBC1798. The S1C5 kappa light chain was PCR amplified from plasmid pLSBC1757 with upstream primer 7659 and downstream primer 6057 (Seq ID No: 6). The S1C5 gamma heavy chain was PCR amplified from plasmid pLSBC2523 with upstream primer 7660 and downstream primer 2227. Separate 100 μL PCR reactions containing 1 μM upstream, 1 μM downstream, 1×Expand High Fidelity Buffer with MgCl₂, 0.2 mM dATP, 0.2 mM dCTP, 0.2 mM dGTP, 0.2 mM dTTP, 3.5 Units Expand High Fidelity and 1 μL plasmid template were amplified at 94° C. for 1 minute, 30 cycles of 94° C. for 30 seconds, 55° C. for 30 seconds, 72° C. for 1 minute, and a final step of 7 minutes at 72° C.

The amplification of the desired approximately 120 bp KP6 propeptide encoding sequence, 700 bp S1C5 kappa light chain encoding sequence and 1.3 Kb S1C5 gamma heavy chain encoding sequence were confirmed by agarose gel electrophoresis. To assemble of the S1C5 MAb proprotein, the amplified S1C5 kappa light chain, the 1731 KP6 PCR fragment, and the amplified S1C5 gamma heavy chain were fused by sequence overlap extension (SOE).

A 25 μL PCR reaction containing 0.1 μL pLSBC1731 PCR fragment, 0.1 μL PCR amplified S1C5 gamma heavy chain, 0.1 μL PCR amplified S1C5 kappa light chain, 1×Expand High Fidelity buffer with MgCl₂, 0.2 mM dATP, 0.2 mM dCTP, 0.2 mM dGTP, 0.2 mM dTTP, 3.5 Units Expand High Fidelity enzyme was amplified at 97° C. for 1 minute, 15 cycles of 94° C. for 30 seconds, 55° C. for 2 minutes, 72° C. for 90 seconds and a final step of 72° C. for 5 minutes. The PCR reaction was purified using the MinElute PCR purification kit (Qiagen) following the manufacturers instructions. Briefly, 5 volumes of PB buffer was added to the reaction, mixed, applied to the column and centrifuged at 14K rpm for 1 minute. The column was washed with 750 μL Buffer PE and the purified fragment eluted in 10 μL EB. A 50 μL reaction containing 5 μL purified PCR product, 50 mM potassium acetate, 20 mM Tris-Acetate pH 7.9, 1 mM DTT, 10 mM magnesium acetate, 20 Units NgoMIV and 8 Units Avr II was incubated at 37° C. for 1 hour and electrophoresed on a 1.0% agarose gel with TAE and 0.5 μg/mL ethidium bromide to separate the approximately 2.3 Kb S1C5 MAb proprotein encoding sequence. The 2.3 Kb MAb proprotein encoding sequence was purified using the QIAquick gel extraction kit (Qiagen) following manufacturers recommendations and eluted with 50 μL EB Buffer.

The 2.3 Kb NgoMVI and Avr II digested fragment of S1C5 MAb proprotein encoding fragment was ligated into the NgoMIV and Avr II prepared pLSBC1767 plasmid to create pLSBC1798. A 30 μL ligation reaction containing 23 μL prepared NgoM VI and Avr II S1C5 MAb prepared PCR fragment, 0.4 μg NgoM VI and Avr II pLSBC1767 fragment, 1200 Units T4 DNA Ligase, 50 mM Tris-HCl (pH 7.5), 10 mM MgCl₂, 25 μg/mL BSA, 10 mM DTT, 1 mM ATP was incubated at 14° C. overnight. The ligation reaction was ethanol precipitated and the pellet was resuspended in 10 μL water and 2 μL used to transform electrocompetent JM109 as previously described. Cells were plated on LB plates containing 50 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and plasmid eluted with 100 μL EB buffer. Clones of pLSBC1798 were confirmed to contain the 2.3 Kb insert and the 9.7 Kb vector fragments by restriction enzyme mapping with NgoMIV and Avr II followed by agarose gel electrophoresis. The S1C5 MAb proprotein was sequenced using standard methods to verify the sequence.

### EXAMPLE 7

PURIFICATION OF 9E10 AND S1C5 MABS

Infectious transcripts were synthesized in-vitro from pLSBC1799 (9E10) and pLSBC1798 (S1C5) clones using the mMessage mMachine T7 kit (Ambion, Austin, Tex.) following the manufacturers directions. Briefly, a 324 μL reaction for each plasmid containing 32 μL 10×Reaction buffer, 162 μL 2×NTP/CAP mix, 32 μL Enzyme mix and 5 μg plasmid was incubated at 37° C. for 2 hours. The synthesized transcripts were encapsidated in a 7 mL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 18 mL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate 25 day post sow Nicotiana benthamiana expressing the TMV 30K movement protein driven by the CaMV 35S promoter and containing the NOS terminator as a transgene was made by standard transformation techniques. High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of MAb protein.

Interstitial fluid from infected leaves of each plant was harvested 8 days post inoculation. Systemically infected upper leaves from each of the infected plants was harvested. The secreted protein fraction, or interstitial fluid (IF) was extracted and analyzed for presence of recombinant protein. The leaf tissue was covered with 50 mM Tris-HCl (pH 7.3), 50 mM NaCl, 2 mM EDTA and subjected to 760 mmHg vacuum for 2 minutes. The vacuum is released and re-applied three times to completely infiltrate the tissue with buffer. The IF fraction was recovered by centrifugation for 20 minutes at 4K rpm. The recovered IF was adjusted to 1 mM PMSF and clarified by centrifugation at 6K rpm for 10 minutes. The supernatant was adjusted to pH 7.5 and 150 mM NaCl, and loaded onto Protein A HiTrap (Amersham Pharmacia) column equilibrated with 150 mM Tris-HCl (pH 7.3), 50 mM NaCl. Bound MAb was eluted with 100 mM Glycine-HCl, pH 3.0 and MAb containing fractions were concentrated approximately 10-fold in Microcon-10 (Amicon) concentrators and diafiltered with phosphate buffered saline (PBS).

### EXAMPLE 8

PURIFICATION OF 9E10 AND S1C5 FAB

Infectious transcripts were synthesized in-vitro from pLSBC1736 (9E10) and pLSBC1792 (S1C5) clones using the mMessage mMachine T7 kit (Ambion, Austin, Tex.) following the manufacturers directions. Briefly, a 100 μL reaction for each plasmid containing 10 μL 10×Reaction buffer, 50 μL 2×NTP/CAP mix, 10 μL Enzyme mix and 1.4 μg plasmid was incubated at 37° C. for 2 hours. The synthesized transcripts were encapsidated in a 2 mL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 5 mL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from each individual clone was used to inoculate 26 day post sow Nicotiana benthamiana. High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of MAb protein.

For pLSBC1736, interstitial fluid from infected leaves of each plant was harvested 12 days post inoculation. Systemically infected upper leaves from each of the infected plants was harvested. The secreted protein fraction, or interstitial fluid (IF) was extracted and analyzed for presence of recombinant protein. The leaf tissue was covered with 50 mM Tris-HCl (pH 7.3), 50 mM NaCl, 2 mM EDTA and subjected to 760 mmHg vacuum for 2 minutes. The vacuum is released and re-applied three times to completely infiltrate the tissue with buffer. The IF fraction was recovered by centrifugation for 20 minutes at 4K rpm. The recovered IF was adjusted to 1 mM PMSF and clarified by centrifugation at 6K rpm for 10 minutes. The supernatant was adjusted to pH 5.2 and then concentrated using a 10 kDa membrane and diafiltered prior to loading on a SP Sepharose FF (Amersham Pharmacia) column, equilibrated with 25 mM Imidazole Buffer, pH 6.0. Bound Fab protein was eluted using a linear gradient of 250-500 mM NaCl in 25 mM Imidazole Buffer, pH 6.0. Eluted fractions were pooled and dialyzed with 10 mM KPO₄ Buffer, pH 6.0 and loaded onto Hydroxyapatite Type I resin (BioRad). Bound protein was eluted using a linear gradient of 10-200 mM KPO₄ Buffer, pH 6.0 and flow through fractions containing purified Fab were pooled together, concentrated and diafiltered into Phosphate Buffered Saline (PBS), pH 7.4.

For pLSBC1792, interstitial fluid from infected leaves of each plant was harvested 12 days post inoculation. Systemically infected upper leaves from each of the infected plants was harvested. The secreted protein fraction, or interstitial fluid (IF) was extracted and analyzed for presence of recombinant protein. The leaf tissue was covered with 50 mM Tris-HCl (pH 7.3), 50 mM NaCl, 2 mM EDTA and subjected to 760 mmHg vacuum for 2 minutes. The vacuum is released and re-applied three times to completely infiltrate the tissue with buffer. The IF fraction was recovered by centrifugation for 20 minutes at 4K rpm. The recovered IF was adjusted to 1 mM PMSF and clarified by centrifugation at 6K rpm for 10 minutes. The supernatant was adjusted to pH 5.2 and then concentrated using a 10 kDa membrane and diafiltered prior to loading on a SP Sepharose FF (Amersham Pharmacia) column, equilibrated with 25 mM Imidazole Buffer, pH 6.0. Bound Fab protein was eluted using a linear gradient of 250-500 mM NaCl in 25 mM Imidazole Buffer, pH 6.0. Eluted fractions were adjusted to 25% ammonium sulfate and loaded onto Phenyl Sepharose HP (Amersham Pharmacia) and Fab protein was eluted using a linear gradient of 20% - 0% (NH₄)₂SO₄ in 25 mM Imidazole Buffer, pH 6.0. Eluted fractions were pooled and dialyzed with 10 mM KPO₄ Buffer, pH 6.0 and loaded onto Hydroxyapatite Type I resin (BioRad). Bound protein was eluted using a linear gradient of 10-200 mM KPO₄ Buffer, pH 6.0 and flow through fractions containing purified Fab were pooled together, concentrated and diafiltered into Phosphate Buffered Saline (PBS), pH 7.4.

### EXAMPLE 9

ANALYSIS OF PURIFIED 9E10 MAB AND FAB

Purified pLSBC1799 MAb samples were prepared for SDS-PAGE analysis by the addition of 5×tris-glycine sample dye containing 10% 2-mercaptoethanol, for reducing gels, and then boiled for 2 minutes. Samples were separated on a 10-20% gradient gel (Novex) and the proteins were detected by Coomassie R-250 Brilliant blue staining. Protein banding in the reducing gel at approximately 50 KDa and 25 KDa indicates the presence of the desired 50 KDa heavy chain and the 25 KDa light chain.

Purified MAb samples were prepared for SDS-PAGE analysis by the addition of 5×tris-glycine sample dye without 2-mercaptoethanol, for non-reducing gels, and then boiled for 2 minutes. Samples were separated on a 6% gradient gel (Novex) and the proteins were detected by Coomassie R-250 Brilliant blue staining. Protein banding in the reducing gel at approximately 150 KDa band under non-reducing conditions indicating the presence of assembled 9E10 MAb protein containing interchain disulfide bridges.

The samples were subjected to western blot analysis to verify the presence of the assembled, disulfide linked heavy chain and light chain polypeptides. Purified MAb samples were prepared for SDS-PAGE analysis by the addition of 5×Tris-glycine sample dye containing 10% 2-mercaptoethanol, for reducing gels, and then boiled for 2 minutes. Samples were loaded on two separate Novex 6% tris glycine gels and subsequently transferred to Nitrocellulose membrane using the Xcell II Blot (Invitrogen, Carlsbad, Calif.) following manufacturers instructions. The membranes were blocked overnight in TBST containing 2.5% powdered skim milk and 2.5% BSA. The first membrane was probed with a 1:4000 dilution of Goat anti-mouse kappa-HRP labeled sera and the second membrane was probed with 1:4000 dilution of Goat anti-mouse IgG-HRP labeled sera (Southern Biotechnology, Birmingham, Ala.) for 1 hour at room temperature. The blots were washed three times in TBST and the labeled proteins detected with the ECL+plus Western Blotting Detection System (Amersham Biosciences, Buckinghamshire, England). The anti kappa sera detected an approximately 150 KD band under non-reducing conditions indicating the presence of assembled 9E10 MAb protein containing interchain disulfide bridges. The anti gamma sera detected an approximately 150 KD band under non-reducing conditions indicating the presence of assembled 9E10 MAb protein containing interchain disulfide bridges.

To verify the ability of the pLSBC1799 produced MAb and pLSBC1736 Fab to recognize the c-myc peptide, purified, plant produced MAb and Fab were used to detect myc-tagged protein by ELISA. Control 9E10 MAb was purified from mouse hybridoma cell line Myc 1-9E10.2 (ATCC (CRL-1729)). Cells were cultured under standard conditions and antibody purified from 90 mL of media using the IgG Protein A Purification Kit (Pierce) following manufacturers directions. Maxisorp ELISA plates (Nunc) were coated overnight at 4° C. with 5 ug/ml antigen in 50 mM Sodium carbonate buffer (pH 9.6). The antigen was the fusion protein from pLSBC2268 (Seq ID No: 95), which contains the c-myc epitope fused to the amino terminus of TMV-U1 coat protein. The plates were blocked with 2.5% BSA in 1×TBST buffer for 1 hour at room temperature. Duplicate samples were tested for the MAb dilutions and Fab dilutions, which were added to the plates and incubated for an hour at room temperature. Plates were washed with TBST, and bound antibody detected with goat anti-mouse kappa HRP (Southern Biotech). Samples were detected with Turbo-TMB ELISA, 1-STEP (Pierce) and the reaction was stopped with the addition of 1N H₂SO₄ following manufacturers instructions. Plates were read at 450 nm by an absorbance plate reader (Molecular Devices) and the data was analyzed with SoftMax software (Molecular Dynamics). Sample data have background subtracted. The ELISA assay demonstrates the LSBC1736 Fab and LSBC1799 MAb recognize and bind to the c-myc antigen, and this activity is comparable to the hybridoma produced control MAb.

| | LSBC1799 MAb | LSBC1799 MAb |
|---|---|---|
| ng | A450 | A450 |
| 88.00 | 0.416 | 0.391 |
| 44.00 | 0.379 | 0.36 |
| 22.00 | 0.322 | 0.32 |
| 11.00 | 0.266 | 0.251 |
| 5.50 | 0.18 | 0.184 |
| 2.75 | 0.118 | 0.125 |
| 1.38 | 0.073 | 0.074 |
| 0.69 | 0.042 | 0.043 |
| 0.34 | 0.022 | 0.025 |
| 0.17 | 0.005 | 0.014 |
| 0.09 | −0.001 | 0.009 |
| 0.04 | 0.001 | −0.001 |

| | LSBC1736 Fab | LSBC1736 Fab |
|---|---|---|
| Ng | A450 | A450 |
| 1100.00 | 0.398 | 0.407 |
| 550.00 | 0.395 | 0.394 |
| 275.00 | 0.395 | 0.404 |
| 137.50 | 0.382 | 0.383 |
| 68.75 | 0.374 | 0.382 |
| 34.38 | 0.35 | 0.358 |
| 17.19 | 0.329 | 0.337 |
| 8.59 | 0.273 | 0.283 |
| 4.30 | 0.216 | 0.214 |
| 2.15 | 0.151 | 0.148 |
| 1.07 | 0.093 | 0.096 |
| 0.54 | 0.054 | 0.056 |

| | 9E10 Control MAb | 9E10 Control MAb |
|---|---|---|
| Ng | A450 | A450 |
| 85.00 | 0.417 | 0.405 |
| 42.50 | 0.367 | 0.347 |
| 21.25 | 0.315 | 0.316 |
| 10.63 | 0.254 | 0.24 |
| 5.31 | 0.177 | 0.191 |
| 2.66 | 0.11 | 0.126 |
| 1.33 | 0.073 | 0.079 |
| 0.66 | 0.042 | 0.048 |
| 0.33 | 0.025 | 0.028 |
| 0.17 | 0.015 | 0.019 |
| 0.08 | 0.01 | 0.013 |
| 0.04 | 0.008 | 0.01 |

### EXAMPLE 10

ANALYSIS OF PURIFIED S1C5 MAB AND FAB

Purified pLSBC1798 MAb samples were prepared for SDS-PAGE analysis by the addition of 5×tris-glycine sample dye containing 10% 2-mercaptoethanol, for reducing gels, and then boiled for 2 minutes. Samples were separated on a 10-20% gradient gel (Novex) and the proteins were detected by Coomassie R-250 Brilliant blue staining. Protein banding in the reducing gel at approximately 50 KDa and 25 KDa indicates the presence of the desired 50 KDa heavy chain and the 25 KDa light chain.

The samples were subjected to western blot analysis to verify the presence of the heavy chain and light chain polypeptides. Purified MAb samples were prepared for SDS-PAGE analysis by the addition of 5×tris-glycine sample dye containing 10% 2-mercaptoethanol, for reducing gels, and then boiled for 2 minutes. Samples were loaded on two separate Novex 10-20% tris glycine gels and subsequently transferred to Nitrocellulose membrane using the Xcell II Blot (Invitrogen, Carlsbad, Calif.) following manufacturers instructions. The membranes were blocked overnight in TBST containing 2.5% powdered skim milk and 2.5% BSA. The first membrane was probed with a 1:4000 dilution of Goat anti-mouse kappa-HRP labeled sera and the second membrane was probed with 1:4000 dilution of Goat anti-mouse IgG-HRP labeled sera (Southern Biotechnology, Birmingham, Ala.) for 1 hour at room temperature. The blots were washed three times in TBST and the labeled proteins detected with the ECL+plus Western Blotting Detection System (Amersham Biosciences, Buckinghamshire, England). The anti kappa sera detected an approximately 25 KDa proteins and a corresponding approximately 50 KDa protein was detected with the anti gamma sera indicating that both the kappa light and gamma heavy chains were expressed, processed and secreted.

Purified MAb samples were prepared for SDS-PAGE analysis by the addition of 5×tris-glycine sample dye without 2-mercaptoethanol, for non-reducing gels, and then boiled for 2 minutes. Samples were separated on a 6% gradient gel (Novex) and the proteins were detected by Coomassie R-250 Brilliant blue staining. Protein banding in the reducing gel at approximately 150 KDa band under non-reducing conditions indicating the presence of assembled S1C5 MAb protein containing interchain disulfide bridges.

The samples were subjected to western blot analysis to verify the presence of the assembled, disulfide linked heavy chain and light chain polypeptides. Purified MAb samples were prepared for SDS-PAGE analysis by the addition of 5×tris-glycine sample dye containing 10% 2-mercaptoethanol, for reducing gels, and then boiled for 2 minutes. Samples were loaded on two separate Novex 6% tris glycine gels and subsequently transferred to Nitrocellulose membrane using the Xcell II Blot (Invitrogen, Carlsbad, Calif.) following manufacturers instructions. The membranes were blocked overnight in TBST containing 2.5% powdered skim milk and 2.5% BSA. The first membrane was probed with a 1:4000 dilution of Goat anti-mouse kappa-HRP labeled sera and the second membrane was probed with 1:4000 dilution of Goat anti-mouse IgG-HRP labeled sera (Southern Biotechnology, Birmingham, Ala.) for 1 hour at room temperature. The blots were washed three times in TBST and the labeled proteins detected with the ECL+plus Western Blotting Detection System (Amersham Biosciences, Buckinghamshire, England). The anti kappa sera detected an approximately 150 KD band under non-reducing conditions indicating the presence of assembled S1C5 MAb protein containing interchain disulfide bridges. The anti gamma sera detected an approximately 150 KD band under non-reducing conditions indicating the presence of assembled S1C5 MAb protein containing interchain disulfide bridges.

To verify the ability of the pLSBC1798 produced MAb and pLSBC1792 produced Fab to recognize the 38C13 antigen (McCormick et al. (1999) Proc. Natl. Acad. Sci. USA. 96:703-708), purified, plant produced MAb and Fab were used to detect 38C13 scFv protein by ELISA. Control S1C5 MAb was from mouse ascites fluid produced using standard techniques and control S1C5 Fab was produced from mouse ascites MAb using the ImmunoPure Fab Kit (Pierce). Maxisorp ELISA plates (Nunc) were coated overnight at 4° C. with 5 ug/ml 38C13 scFv in 50 mM Sodium carbonate buffer (pH 9.6). The plates were blocked with 2.5% BSA in IX TBST buffer for 1 hour at room temperature. Duplicate samples were tested for the MAb dilutions and Fab dilutions, which were added to the plates and incubated for an hour at room temperature. Plates were washed with TBST, and bound antibody detected with goat anti-mouse kappa HRP (Southern Biotech). Samples were detected with Turbo-TMB ELISA, 1-STEP (Pierce) and the reaction was stopped with the addition of 1N H₂SO₄ following manufacturers instructions. Plates were read at 450 nm by an absorbance plate reader (Molecular Devices) and the data was analyzed with SoftMax software (Molecular Dynamics). Sample data have background subtracted. The ELISA assay demonstrates the LSBC1792 Fab and LSBC1798 MAb recognize and bind to the 38C13 antigen, and this activity is comparable to the ascites produced control Fab and MAb.

| | | | S1C5 | S1C5 |
|---|---|---|---|---|
| Ng | LSBC1792 Fab | LSBC1792 Fab | Control Fab | Control Fab |
| 50.000 | 0.524 | 0.504 | 0.481 | 0.516 |
| 25.000 | 0.521 | 0.519 | 0.498 | 0.51 |
| 12.500 | 0.487 | 0.485 | 0.46 | 0.465 |
| 6.250 | 0.468 | 0.449 | 0.346 | 0.363 |
| 3.125 | 0.366 | 0.35 | 0.266 | 0.273 |
| 1.563 | 0.263 | 0.247 | 0.168 | 0.171 |
| 0.781 | 0.168 | 0.154 | 0.095 | 0.094 |
| 0.391 | 0.09 | 0.094 | 0.047 | 0.05 |
| 0.195 | 0.049 | 0.041 | 0.03 | 0.023 |
| 0.098 | 0.025 | 0.019 | 0.009 | 0.018 |
| 0.049 | 0.01 | 0.011 | 0.008 | 0.003 |

| | LSBC1798 | LSBC 1798 | S1C5 | S1C5 |
|---|---|---|---|---|
| ng | MAb | MAb | Control MAb | Control MAb |
| 150.000 | 0.521 | 0.52 | 0.517 | 0.538 |
| 75.000 | 0.514 | 0.517 | 0.532 | 0.515 |
| 37.500 | 0.477 | 0.482 | 0.443 | 0.497 |
| 18.750 | 0.39 | 0.397 | 0.476 | 0.429 |
| 9.375 | 0.295 | 0.307 | 0.39 | 0.395 |
| 4.688 | 0.198 | 0.194 | 0.283 | 0.284 |
| 2.344 | 0.106 | 0.113 | 0.18 | 0.179 |
| 1.172 | 0.061 | 0.061 | 0.105 | 0.096 |
| 0.586 | 0.03 | 0.029 | 0.053 | 0.047 |
| 0.293 | 0.014 | 0.015 | 0.025 | 0.023 |
| 0.146 | 0.007 | 0.004 | 0.012 | 0.01 |

### EXAMPLE 11

Cloning of the 4d5 Heavy Chain fd and Light Chain Genes

The murine monoclonal antibody mumAb4D5 is directed against the extracellular domain of HER-2/neu gene product p185^{HER2} and it specifically inhibits the growth of cells of the breast cancer cell line SK-BR-3 (ATCC HTB 30) in 6 day culture. Such treatment sensitizes these cells to chemotherapeutic agents (U.S. Pat. 5,677,171). The process of Example 4 is repeated using the Ig heavy Fd region and the light chain of the mumAb4D5. The variable gene sequences of the immunoglobulin coding sequences are described in Carter et al., PNAS 89:4285-4289 (1992).

Mouse hybridoma line A-HER2 expressing murine monoclonal antibody (IgG1) described in Fendly et al., Cancer Res.50: 1550-1558(1990) which recognizes the extracellular domain of human HER-2 receptor was obtained from ATCC. Cells were cultured following the instructions supplied with the cell line. The heavy chain Fd region and kappa light chain genes were isolated by PCR amplification of mRNA from the hybridoma. Briefly, 1×10⁷ cultured cells were spun and washed to remove excess culture media and lysed with 600 μL RLT buffer containing 1% 2-mercaptoethanol (Qiagen, Valencia, Calif.). Total RNA was purified using the QIAshredder and RNEASY column per manufacturers directions. Briefly, the cell lysate was applied to the QIAshredder column and spun in a centrifuge for 2 minutes at 14K rpm. The flow through was collected and diluted with an equal volume of 70% ethanol. The mixture was transferred to a RNeasy column and centrifuged for 15 seconds at 10K rpm until all sample was processed through the column. The RNA bound to the column was washed with 700 μL RW1 followed by a wash with 500 μL RPE and subsequently dried. The purified RNA was eluted in 50 μL RNASE free water by centrifugation for 1 minute at 10K rpm. 4 μg of the above prepared total RNA was incubated at 65° C. for 2 minutes, immediately placed on ice for 3 minutes, and then applied to 20 μL of magnetic beads in binding buffer (20 mM Tris-HCl (pH 7.5), 1.0 M LiCl, 2 mM EDTA) where the beads were prepared by washing with 50 μL of binding buffer. The RNA and bead mixture were incubated for 5 minutes with constant rotating. The supernatant containing unbound material was removed and the beads were washed with 100 μL washing buffer (10 mM Tris-HCl (pH 7.5), 0.15 M LiCl, 1 mM EDTA) followed by the addition of 40 μL nuclease free water. cDNA was synthesized in 60 μL reactions containing 50 mM Tris HCl (pH 8.3), 75 mM KCl, 3 mM MgCl₂, 10 mM DTT, 2 Units RNasin (Promega, Madison, Wis.), 20 Units Superscript II (Invitrogen, Carlsbad, Calif.), 0.5 mM dATP, 0.5 mM dCTP, 0.5 mM dGTP, 0.5 mM dTTP, and the oligo dT bound RNA from above. The cDNA reaction was incubated at 42° C. for 60 minutes with constant rotation.

Heavy chain Fd genes were PCR amplified using a gene specific upstream primer which anneals to the 5′ end of the framework 1 region (FR1) of heavy chain gene 4D5 HySph5′ (Seq ID No: 42) and a C_{H}1 specific 3′ downstream primer 4D5 Hy Avr3′ (Seq ID No: 52). The kappa light chain gene was PCR amplified in a separate reaction with a gene specific upstream primer which anneals to the 5′ end of the framework 1 region (FR1) of kappa light chain gene 4D5 LtSph5′ (Seq ID No: 43) and a C_{L} specific 3′ downstream primer 4D5 Lt Avr3′ (Seq ID No: 53). 50 μL PCR reactions contained 1×Expand High Fidelity buffer with MgCl₂, 0.2 mM dATP, 0.2 mM dCTP, 0.2 mM dGTP, 0.2 mM dTTP, 3.5 Units Expand High Fidelity Polymerase, 0.4 μM upstream primer, 0.4 μM downstream primer and 2 μL prepared cDNA. PCR reactions were amplified at 97° C. for 1 minute, 30 cycles of 94° C. for 30 seconds, 48° C. for 30 seconds, 72° C. for 30 seconds, and 5 minutes at 72° C. The amplification of the desired approximately 700 bp kappa light chain and the approximately 700 bp gamma Fd region were confirmed by agarose gel electrophoresis. The above PCR reactions were precipitated with 3 volumes ethanol and 0.3 volumes 10M NH₄Acetate, spun and washed with 70% ethanol. The pellets were resuspended in 20 μL 10 mM Tris-HCL (pH 8.0).

The prepared PCR fragments from above were cloned into pCR4-TOPO (Invitrogen) following the manufacturers directions to create plasmid p4D5Hy-TOPO (Seq ID No: 81) and p4D5Lt-TOPO (Seq ID No: 83). Briefly, 2 μL of PCR product, 1 μL vector, 1 μL of salt solution and 1 μL of water were mixed, incubated at room temperature for 5 minutes. The ligations were placed on ice and 25 μL of chemically competent Top 10 cells was added to each ligation and the mixes were incubated on ice for 10 minutes. The transformation reactions were heat shocked by incubating at 42° C. for 30 seconds and immediately placed on ice and 250 μL of SOC was added. The transformations were allowed to recover by incubating at 37° C., 200 rpm shaking for 20 minutes. The transformations were plated out on LB plates containing ampicillin and grown overnight at 37° C. Individual colonies were used to inoculate 1.0 mL Super Broth (SB) containing 100 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.). Briefly, the cells were pelleted by centrifugation at 3 K rpm for 15 minutes in a plate centrifuge. The supernatant was drained from the cell pellets and the cells resuspended in 250 μL P1 Buffer by vortexing. 250 μL of P2 was added to the cells, mixed by inverting and incubated for 5 minutes to lyse the cells. 350 μL of N3 was added to the cell lysates, mixed by inverting and transferred to the Turbo Filter plate. A vacuum was applied to the Turbo Filter which filtered the sample into the QIAprep plate. A vacuum was then applied to the QIAprep plate pulling the sample through the plate and bound the plasmid to the plate membrane. The QIAprep plate was washed using vacuum force with 0.9 mL of PB, followed by two washes with 0.9 mL of PE and vacuum dried. 100 μL EB buffer was added to the purified plasmid, incubated for 1 minute, and subsequently centrifuged for 3 minute at 6K rpm to elute the purified plasmid. The presence of the approximately 700 bp insert for each plasmid was verified with Sph I and Avr II restriction digest and agarose gel electrophoresis. The purified p4D5Hy-TOPO (Seq ID No: 81) and p4D5Lt-TOPO (Seq ID No: 83) plasmids were subjected to nucleic acid sequencing with the M13 forward and reverse primers using standard methods to verify the mum4D5 Fd and kappa chain sequences.

### EXAMPLE 12

Cloning of the 4D5 Fab Heavy and Light Chain Proprotein and Expression Analysis

The KP6 sequence of pLSBC1731 was PCR amplified for Fab cloning. A 25 μL PCR reaction containing 0.8 μM 5228, 0.8 μM 5229, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase and 0.03 μL pLSBC1731 plasmid. The PCR reaction was amplified at 97° C. for 1 minute, 15 cycles of 94° C. for 30 seconds, 55° C. for 30 seconds, 72° C. for 30 seconds, and 5 minutes at 72° C. The amplification of the desired approximately 120 bp KP6 propeptide encoding sequence was confirmed by agarose gel electrophoresis. The 4D5 Fd region (V_{H}C_{H}1) was PCR amplified from plasmid p4D5HyFd with upstream primer 4D5 HySph5′ which contains a Sph I site compatible for cloning into vector p1324-MBP which contains the alpha-amylase signal peptide and downstream primer 4D5HyKp63′ (Seq ID No: 44) which contains sequence coding for the 3′ end of the C_{H}1 fused to the 5′ end of the KP6 propeptide sequence amplified from pLSBC1731. p1324-MBP, a modified 30B vector (Shivprasad, S. et al. (1999) Virology 255:312-323), containing a hybrid fusion of TMV and TMGMV-U5 as well as the rice α amylase signal peptide with Sph I and Avr II insert cloning site. In this vector, a TMV coat protein subgenomic promoter is located upstream of the insertion site of the 4D5 Fab proprotein sequence. Following infection, this TMV coat protein subgenomic promoter directs initiation of the 4D5 Fab proprotein RNA synthesis in plant cells at the transcription start point (“tsp”). The rice α amylase signal peptide (O'Neill, S D et al. (1990) Mol. Gen. Genet. 221:235-244), fused in-frame to the 4D5 Fab proprotein sequence, encodes a 31 residue polypeptide which targets proteins to the secretory pathway (Firek, S. et al. (1994) Transgenic Res. 3:326-33 1), and is subsequently cleaved off between the C-terminal Gly of the signal peptide and the N-terminal Met of the expressed 4D5 Fab proprotein. The sequence encoding 4D5 Fab proprotein has been introduced between the 30K movement protein and the TMGMV-U5 coat protein (Tcp) genes. A T7 phage RNA Polymerase promoter has been introduced upstream of the viral cDNA, allowing for transcription of infective genomic plus-strand RNA. The Sph I site joins the signal peptide to the FR1 of the 4D5 variable region of the Fd and directs the secretion of the artificial proprotein to the ER. The 4D5 light chain (V_{L}C_{L}) was PCR amplified from the plasmid p4D5Lt with downstream primer 4D5Lavstp3′ (Seq ID No: 49) which contains a translation termination codon at the 3′ end of the C_{L} coding sequence followed by an Avr II site compatible for cloning into vector p1324-MBP and upstream primer 4D5LtKp65′ (Seq ID No: 45) which contains sequence coding for the 3′ end of the KP6 propeptide sequence amplified from pLSBC1731 fused to the 5′ end of the FR1 region of the V_{L} coding sequence. The 4D5 Fd and light chain regions were PCR amplified in separate 25 μL PCR reactions containing 0.8 μM upstream primer, 0.8 μM downstream, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase and 0.03 μL plasmid template. The PCR reaction was amplified at 97° C. for 1 minute, 15 cycles of 94° C. for 30 seconds, 55° C. for 30 seconds, 72° C. for 30 seconds, and 5 minutes at 72° C. The amplification of the desired approximately 700 bp Fd and light chain fragments were verified by agarose gel electrophoresis. To assemble of the 4D5 Fab proprotein the KP6 PCR fragment was fused to the amplified Fd fragment and the amplified light chain fragment by sequence overlap extension (SOE). To assemble the 4D5 proprotein, a 25 μL PCR reaction containing 0.03μL pLSBC1731 PCR product from above, 0.03μL p4D5Lt PCR product from above, 0.03μL p4D5HyFd PCR product from above, 0.8 μM 4D5 HySph5′ upstream primer, 0.8 μM 4D5Lavstp3′ downstream primer, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase. The PCR reaction was amplified at 97° C. for 1 minute, 15 cycles of 94° C. for 30 seconds, 55° C. for 2 minutes, 72° C. for 30 seconds, and 5 minutes at 72° C. The amplification of the desired approximately 1.4 Kb 4D5 Fab proprotein was verified by agarose gel electrophoresis.

A phenol chloroform extraction series was performed on the PCR amplified product to remove the thermostable polymerase prior to restriction digestion. 5 μL of the prepared fragment was digested with Sph I and Avr II in a 25 uL reaction containing 2.5 Units Sph I, 2 Units Avr II, 50 mM NaCl, 10 mM Tris-HCl (pH 7.9), 10 mM MgCl₂, 1 mM DTT. The digest was incubated at 37° C. for 2 hours, and electrophoresed on a 1.0% agarose gel to separate the approximately 1.4 Kb fragment. The nucleic acids were stained with GelStar (Cambrex Bio Science) and the approximately 1.4 Kb fragment was isolated. The fragment was purified away from the agarose using QIAquick gel extraction kit following the manufacturers instructions. The recovery of the Sph I/Avr II digested fragment was verified by gel electrophoresis. The 1.4 Kb Sph I and Avr II 4D5 Fab proprotein was cloned into the SphI and Avr II prepared p1324-MBP plasmid to create pLSBC1740 (Seq ID No: 71). A 50 μL ligation reaction containing 10 μL prepared 4D5 Fab proprotein, 0.4 μg p1324-MBP, 800 Units T4 DNA Ligase, 50 mM Tris-HCl (pH 7.5), 10 MM MgCl₂, 25 μg/mL BSA, 10 mM DTT, 1 mM ATP was incubated at 14° C. overnight. The ligation was precipitated with 3 volumes ethanol and 0.3 volumes 10M NH₄Acetate, spun and washed with 70% ethanol. The pellets were resuspended in 6 μL 10 mM Tris-HCL (pH 8.0). Bacterial transformation was performed with a Gene Pulser electroporator (BioRad, Hercules, Calif.) following manufacturer recommendations. Briefly, 40 μL of electro-competent JM109 cells were mixed with 2 μL of ligation and transferred to a cold 0.2 cm cuvette. The mixture was pulsed at 2.5 KV, 200 ohms, 25 μFD. After pulsing, 200 μL of SOC was added and the cells allowed to recover for 20 minutes at 37° C. Cells were plated on LB plates containing 50 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and eluted with 100 μL EB buffer. Clones were confirmed to contain the 1.4 Kb insert and the 9.7 Kb vector fragments by restriction enzyme mapping with Sph I and Avr II followed by agarose gel electrophoresis. The 4D5 Fab proprotein was sequenced using standard methods to verify the sequence.

Infectious transcripts were synthesized in-vitro from the pLSBC1740 (Seq ID No: 71) clone using the mMessage mMachine T7 kit (Ambion, Austin, Tex.) following the manufacturers directions. Briefly, a 5.5 μL reaction containing 1 μL 10×Reaction buffer, 2.5 μL 2×NTP/CAP mix, 1 μL Enzyme mix and 3.5 μL plasmid was incubated at 37° C. for 2 hours. The synthesized transcripts were encapsidated in a 40 μL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 40 μL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate a 19 day post sow Nicotiana benthamiana plant (Dawson, WO et al. (1986) Proc. Natl. Acad Sci. USA 83:1832-1836). High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of Fab protein.

Interstitial fluid from infected leaves of each plant was harvested 9 days post inoculation and screened by ELISA. Systemically infected upper leaves from individual plants were harvested. The secreted protein fraction, or interstitial fluid (IF) was extracted and analyzed for presence of recombinant protein. The leaf tissue was placed in a GF/B 0.8 mL Unifilter (Whatman, Clifton, N.J.), covered with 20 mM Tris-HCl (pH 7.0) and subjected to 760 mmHg vacuum for 30 seconds. The vacuum is released and re-applied three times to completely infiltrate the tissue with buffer. The residual buffer is discarded and the tissue dried by centrifugation at 400 rpm in a plate centrifuge for 30 seconds. The IF fraction is recovered into a 96-well microplate by centrifugation for 10 minutes at 3K rpm in a plate centrifuge. Each sample was analyzed by ELISA in triplicate. 6 μL of IF is adjusted to 50 mM Na₂CO₃ pH 9.6 in 100 μL and applied to a 96 well plate (Maxisorb, Nunc) and incubated overnight at 4° C. Plates were blocked with 200 μL of 1% BSA in PBS for 30 minutes at 37° C. followed by washing four times with 150 mM NaCl, 0.05% Trition X-100. Plates were incubated with 100 μL of a 1:4000 dilution of goat anti-mouse kappa serum conjugated with horseradish peroxidase (Southern Biotechnology) in PBS and incubated at room temperature for 1 hour. Plates were washed 4 times with PBST and incubated for 20 minutes at room temperature with 100 μL of Turbo-TMB ELISA, 1-STEP (Pierce). The reaction was stopped with the addition of 50 μL 1N H₂SO₄ and read at 450 nm by an absorbance plate reader (Molecular Devices) and the data was analyzed with SoftMax software(Molecular Dynamics). Samples with a reading greater than 0.13 were further analyzed.

The pLSBC1740 clone was digested with Pac I and Kpn I to isolate the 2.7 Kb alpha-amylase signal peptide, 4D5 Fab proprotein including the viral 3′ end. A 50 μL reaction containing 2 μL of plasmid, 10 Units Pac I, 10 Units Kpn I, 10 mM Bis-Tris Propane-HCl (pH 7.0), 10 mM MgCl₂, 1 mM DTT. The digest was incubated at 37° C. for 2 hours, and electrophoresed on a 1.0% agarose gel to separate the approximately 2.7 Kb fragment. The nucleic acids were stained with GelStar (Cambrex Bio Science) and the approximately 2.7 Kb fragment was isolated. The fragment was purified away from the agarose using QIAquick gel extraction kit following the manufacturers instructions. The recovery of the Pac I/Kpn I digested fragment was verified by gel electrophoresis. The 2.7 Kb Pac I and Kpn I fragment of pLSBC1740 was cloned into the Pac I and Kpn I prepared p1177MP5 plasmid 8.0 Kb fragment to create pLSBC1766 (Seq ID No: 89). A 50 μL ligation reaction containing 10 μL Pac I/Kpn I fragment of pLSBC1740, 0.4 μg p1177MP5, 800 Units T4 DNA Ligase, 50 mM Tris-HCl (pH 7.5), 10 mM MgCl₂, 25 μg/mL BSA, 10 mM DTT, 1 mM ATP was incubated at 14° C. overnight. The ligation was ethanol precipitated as previously described. The pellets were resuspended in 10 mM Tris-HCL (pH 8.0). Bacterial transformation was performed with a Gene Pulser electroporator (BioRad, Hercules, Calif.) following manufacturer recommendations with 40 μL of electro-competent JM109 cells as previously described. Cells were plated on LB plates containing 50 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and plasmid eluted with 100 μL EB buffer. The presence of a 1.4 Kb insert was verified by restriction mapping with Sph I and Avr II followed by agarose gel electrophoresis.

Infectious transcripts were synthesized in-vitro from 300 ng template plasmid in an 11 1μL reaction using the mMessage mMachine T7 kit (Ambion, Austin, Tex.) and the transcripts were encapsidated with purified U1 coat protein as above. Transcripts were used to inoculate and systemically infect 20 day old Nicotiana benthamiana plants and the IF protein fraction was isolated at 8 and 11 days post inoculation by vacuum infiltration and centrifugation as previously described. 20 μL of each IF sample was prepared for SDS-PAGE analysis by the addition of 5 μL 5×tris-glycine sample dye containing 10% 2-mercaptoethanol for reducing gels and no 2-mercaptoethanol for non-reducing gels and the mixture was boiled for 2 minutes. Samples were separated on a 10-20% gradient Criterion gel (Bio-Rad) and the proteins were detected by Coomassie R-250 Brilliant blue staining. Protein banding in the reducing gel at approximately 25 KDa indicates the presence of the desired 25 KDa heavy chain Fd and the 25 KDa light chain. A corresponding protein at approximately 50 KDa under non-reducing conditions as seen as evidence of a assembled, disulfide linked Fab heterodimer consisting of the heavy chain Fd and the kappa light chain.

### EXAMPLE 13

Cloning of the 4D5 Fab Light and Heavy Chain Proprotein and Expression Analysis

The KP6 sequence of pLSBC1731 was PCR amplified for Fab cloning. A 25 μL PCR reaction containing 0.8 μM 5228, 0.8 μM 5229, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase and 0.03 μL pLSBC1731 plasmid. The PCR reaction was amplified at 97° C. for 1 minute, 15 cycles of 94° C. for 30 seconds, 55° C. for 30 seconds, 72° C. for 30 seconds, and 5 minutes at 72° C. The amplification of the desired approximately 120 bp KP6 propeptide encoding sequence was confirmed by agarose gel electrophoresis. The 4D5 light chain (V_{L}C_{L}) was PCR amplified from plasmid p4D5Lt with upstream primer 4D5LtSphI5′ which contains a Sph I site compatible for cloning into vector p1324-MBP which contains the alpha-amylase signal peptide and downstream primer 4D5LtKp63′ (Seq ID No: 46) which contains sequence coding for the 3′ end of the C_{L} fused to the 5′ end of the KP6 propeptide sequence amplified from pLSBC1731. The Sph I site joins the signal peptide to the FR1 of the 4D5 variable region of the light chain and directs the secretion of the artificial proprotein to the ER. The 4D5 Fd heavy chain (V_{H}C_{H}1) was PCR amplified from the plasmid p4D5HyFd with downstream primer 4D5Havstp3′ which contains a translation termination codon at the 3′ end of the C_{H}1 coding sequence followed by an Avr II site compatible for cloning into vector p1324-MBP and upstream primer 4D5HyKp65′ (Seq ID No: 47) which contains sequence coding for the 3′ end of the KP6 propeptide sequence amplified from pLSBC1731 fused to the 5′ end of the FR1 region of the V_{H} coding sequence. The 4D5 Fd and light chain regions were PCR amplified in separate 25 μL PCR reactions containing 0.8 μM upstream primer, 0.8 μM downstream, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase and 0.03 μL plasmid template. The PCR reaction was amplified at 97° C. for 1 minute, 15 cycles of 94° C. for 30 seconds, 55° C. for 30 seconds, 72° C. for 30 seconds, and 5 minutes at 72° C. The amplification of the desired approximately 700 bp Fd and light chain fragments were verified by agarose gel electrophoresis. To assemble of the 4D5 Fab proprotein the KP6 PCR fragment was fused to the amplified Fd fragment and the amplified light chain fragment by sequence overlap extension (SOE). To assemble the 4D5 proprotein, a 25 μL PCR reaction containing 0.03μL pLSBC1731 PCR product from above, 0.03μL p4D5Lt PCR product from above, 0.03μL p4D5HyFd PCR product from above, 0.8 μM 4D5LtSphI5′ upstream primer, 0.8 μM 4D5Havstp3′ (Seq ID No: 48) downstream primer, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase. The PCR reaction was amplified at 97° C. for 1 minute, 15 cycles of 94° C. for 30 seconds, 55° C. for 2 minutes, 72° C. for 30 seconds, and 5 minutes at 72° C. The amplification of the desired approximately 1.4 Kb 4D5 Fab proprotein was verified by agarose gel electrophoresis.

A phenol chloroform extraction series was performed on the PCR amplified product to remove the thermostable polymerase prior to restriction digestion. 5 μL of the prepared fragment was digested with Sph I and Avr II in a 25 uL reaction containing 2.5 Units Sph I, 2 Units Avr II, 50 mM NaCl, 10 mM Tris-HCl (pH 7.9), 10 mM MgCl₂, 1 mM DTT. The digest was incubated at 37° C. for 2 hours, and electrophoresed on a 1.0% agarose gel to separate the approximately 1.4 Kb fragment. The nucleic acids were stained with GelStar (Cambrex Bio Science) and the approximately 1.4 Kb fragment was isolated. The fragment was purified away from the agarose using QIAquick gel extraction kit following the manufacturers instructions. The recovery of the Sph I/Avr II digested fragment was verified by gel electrophoresis. The 1.4 Kb Sph I and Avr II 4D5 Fab proprotein was cloned into the SphI and Avr II prepared p1324-MBP plasmid to create pLSBC1741 (Seq ID No: 73). A 50 μL ligation reaction containing 10 μL prepared 4D5 Fab proprotein, 0.4 μg p1324-MBP, 800 Units T4 DNA Ligase, 50 mM Tris-HCl (pH 7.5), 10 mM MgCl₂, 25 μg/iL BSA, 10 mM DTT, 1 mM ATP was incubated at 14° C. overnight. The ligation was ethanol precipitated as previously described. The pellets were resuspended in 6 μL 10 mM Tris-HCL (pH 8.0). Bacterial transformation was performed with a Gene Pulser electroporator (BioRad, Hercules, Calif.) following manufacturer recommendations with 40 lL of electro-competent JM109 cells as previously described. Cells were plated on LB plates containing 50 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and plasmid eluted with 100 μL EB buffer. Clones were confirmed to contain the 1.4 Kb insert and the 9.7 Kb vector fragments by restriction enzyme mapping with Sph I and Avr II followed by agarose gel electrophoresis. The 4D5 Fab proprotein was sequenced using standard methods to verify the sequence.

Infectious transcripts were synthesized in-vitro from pLSBC1741 clones using the mMessage mMachine T7 kit (Ambion, Austin, Tex.) following the manufacturers directions. Briefly, a 5.5 μL reaction containing 1 μL 10×Reaction buffer, 2.5 μL 2×NTP/CAP mix, 1 μL Enzyme mix and 3.5 μL plasmid was incubated at 37° C. for 2 hours. The synthesized transcripts were encapsidated in a 40 μL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 40 μL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate a 19 day post sow Nicotiana benthamiana plant (Dawson, WO et al. (1986) Proc. Natl. Acad Sci. USA 83:1832-1836). High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of Fab protein.

Interstitial fluid from infected leaves of each plant was harvested 9 days post inoculation and screened by ELISA. Systemically infected upper leaves from individual plants were harvested. The secreted protein fraction, or interstitial fluid (IF) was extracted and analyzed for presence of recombinant protein. The leaf tissue was placed in a GF/B 0.8 mL Unifilter (Whatman, Clifton, N.J.), covered with 20 mM Tris-HCl (pH 7.0) and subjected to 760 mmHg vacuum for 30 seconds. The vacuum is released and re-applied three times to completely infiltrate the tissue with buffer. The residual buffer is discarded and the tissue dried by centrifugation at 400 rpm in a plate centrifuge for 30 seconds. The IF fraction was recovered into a 96-well microplate by centrifugation for 10 minutes at 3K rpm in a plate centrifuge. Each sample was analyzed by ELISA in triplicate. 6 μL of IF is adjusted to 50 mM Na₂CO₃ pH 9.6 in 100 μL and applied to a 96 well plate (Maxisorb, Nunc) and incubated overnight at 4° C. Plates were blocked with 200 μL of 1% BSA in PBS for 30 minutes at 37° C. followed by washing four times with 150 mM NaCl, 0.05% Trition X-100. Plates were incubated with 100 μL of a 1:4000 dilution of goat anti-mouse kappa serum conjugated with horseradish peroxidase(Southern Biotechnology) in PBS and incubated for at room temperature for 1 hour. Plates were washed 4 times with PBST and incubated for 20 minutes at room temperature with 100 μL of Turbo-TMB ELISA, 1-STEP (Pierce). The reaction was stopped with the addition of 50 μL 1N H₂SO₄ and read at 450 nm by an absorbance plate reader (Molecular Devices) and the data was analyzed with SoftMax software(Molecular Dynamics). Samples with a reading greater than 0.13 were further analyzed.

The pLSBC1741 clone was digested with Pac I and Kpn I to isolate the 2.7 Kb alpha-amylase signal peptide, 4D5 Fab proprotein including the viral 3′ end. A 50 μL reaction containing 2μL of plasmid, 10 Units Pac I, 10 Units Kpn I, 10 mM Bis-Tris Propane-HCl (pH 7.0), 10 mM MgCl₂, 1 mM DTT. The digest was incubated at 37° C. for 2 hours, and electrophoresed on a 1.0% agarose gel to separate the approximately 2.7 Kb fragment. The nucleic acids were stained with GelStar (Cambrex Bio Science) and the approximately 2.7 Kb fragment was isolated. The fragment was purified away from the agarose using QIAquick gel extraction kit following the manufacturers instructions. The recovery of the Pac I and Kpn I digested fragment was verified by gel electrophoresis. The 2.7 Kb Pac I and Kpn I fragment of pLSBC1741 was cloned into the Pac I and Kpn I prepared p1177MP5 plasmid 8.0 Kb fragment to create pLSBC1767 (Seq ID No: 91). A 50 μL ligation reaction containing 10 μL prepared of the pLSBC1741, 0.4 μg p1177MP5, 800 Units T4 DNA Ligase, 50 mM Tris-HCl (pH 7.5), 10 mM MgCl₂, 25 μg/mL BSA, 10 mM DTT, 1 mM ATP was incubated at 14° C. overnight. The ligation was ethanol precipitated and used to transform electrocompetent JM109 as previously described. Cells were plated on LB plates containing 50 μg/mL ampicillin and grown overnight at 37° C. Ampicillin resistant colonies were cultured in blocks and plasmid purified using the QIAprep 96 Turbo Miniprep kits (QIAGEN, Valencia, Calif.) as above. The presence of a 1.4 Kb insert was verified by restriction mapping with Sph I and Avr II followed by agarose gel electrophoresis.

Infectious transcripts were synthesized in-vitro from 300 ng template plasmid in an 11 μL reaction using the mMessage mMachine T7 kit (Ambion, Austin, Tex.) and the transcripts were encapsidated with purified U1 coat protein as above. Transcripts were used to inoculate and systemically infect 20 day old Nicotiana benthamiana plants and the IF protein fraction was isolated at 8 and 11 days post inoculation by vacuum infiltration and centrifugation as previously described. 20 μL of each IF sample was prepared for SDS-PAGE analysis by the addition of 5 μL 5×tris-glycine sample dye containing 10% 2-mercaptoethanol for reducing gels and no 2-mercaptoethanol for non-reducing gels and the mixture was boiled for 2 minutes. Samples were separated on a 10-20% gradient Criterion gel (Bio-Rad) and the proteins were detected by Coomassie R-250 Brilliant blue staining. Protein banding in the reducing gel at approximately 25 KDa indicates the presence of the desired 25 KDa heavy chain Fd and the 25 KDa light chain. A corresponding protein at approximately 50 KDa under non-reducing conditions as seen as evidence of a assembled, disulfide linked Fab heterodimer consisting of the heavy chain Fd and the kappa light chain.

### EXAMPLE 14

Cloning of the 4D5 Monoclonal Antibody Proprotein and Expression Analysis

A 4D5 monoclonal antibody artificial proprotein was assembled by fusing the pLSBC1767 4D5 Fab proprotein to the murine gamma 1 immunoglobulin constant domains C_{H}2 and C_{H}3. This fusion will result in a first domain light chain, the second domain propeptide and the third domain the complete heavy chain sequence. The cloned murine IgG1 heavy chain sequence was derived from the previously described p9E10Hy-TOPO clone. The murine IgG1 constant domains genes are conserved within heavy chain genes of the same isotype, therefore the 9E10 C_{H}2 and C_{H}3 are expected to be the same for the 4D5 and 9E10 antibodies as they are both murine IgG1. Primers were designed to amplify the pLSBC1767 fragment using a 5696s (Seq ID No: 54) upstream primer which anneals to vector sequence and the 4D5fAb3′ (Seq ID No: 55) downstream primer which anneals to the C_{H}1 region of pLSBC1767 and removes the translation termination signal. The 4D5fAb3′ downstream primer is designed to anneal to the 3′ end of the pLSBC1767 C_{H}1 region such that treatment with the 3′ to 5′ exonuclease activity of T4 DNA polymerase will result in a “GG” 5′ extension where “G” is guanine. To amplify the C_{H}2 and C_{H}3 sequences of the p9E10Hy-TOPO clone, a 9E10Fc5′ (Seq ID No: 56) upstream primer was designed which anneals to the 5′ end of the C_{H}2 domain such that treatment with the 3′ to 5′ exonuclease activity of T4DNA polymerase will result in a “CC” 5′ extension where “C” is cytosine. The 9E10Havr3′ downstream primer anneals to the 3′ end of the C_{H}3 domain including a translational termination codon followed by an Avr II site for subsequent cloning. Separate 25 μL PCR reaction were set up to amplify the 4D5 Fab and the 9E10 C_{H}2C_{H}3 domain which contained 0.8 μM 5′ primer, 0.8 μM 3′ primer, 1×Expand High Fidelity Buffer with MgCl₂, 0.16 mM dATP, 0.16 mM dCTP, 0.16 mM dGTP, 0.16 mM dTTP, 1.8 Units Expand High Fidelity Polymerase and 0.03 μL plasmid template. The PCR reaction was amplified at 95° C. for 2 minutes, 15 cycles of 95° C. for 30 seconds, 55° C. for 30 seconds, 72° C. for 1 minute, and 7 minutes at 72° C. The amplification of the desired approximately 1.6 Kb 4D5 sequence and the approximately 500 bp 9E10 C_{H}2C_{H}3 were confirmed by agarose gel electrophoresis. The PCR amplified 1.6 Kb 4D5 sequence and the 500 bp 9E10 C_{H}2C_{H}3 were digested with Dpn I. 5 Units Dpn I was added to each PCR reaction and incubated at 37° C. for 1 hour followed by 80° C. for 20 minutes. A phenol chloroform extraction series was performed on the PCR amplified product to remove the thermostable polymerase and the fragment were ethanol precipitated as described earlier and resuspended in 20 μL 10 mM Tris-HCl pH 8. The purified PCR amplified fragments were ligated together in a 30 μL ligation reaction containing 6 μL 4D5 Fab PCR fragment, 2 μL 9E10 C_{H}2C_{H}3 PCR fragment, 50 mM Tris-HCl (pH 7.5), 10 mM MgCl₂, 25 μg/mL BSA, 10 mM DTT, 0.2 mM dTTP, 0.2 mM dATP, 1 MM ATP, 0.6 Units T4 DNA Polymerase, 1.2 Units T4 DNA Ligase, 1.2 Units T4 Polynucleotide Kinase. The reaction was incubated at 23° C. for 1 hour and then heat killed at 75° C. for 15 minutes. The reaction was phenol chloroform extracted to remove the enzymes and the fragment were ethanol precipitated as described earlier and resuspended in 25 μL of 50 mM potassium acetate, 20 mM Tris-Acetate pH 7.9, 1 mM DTT, 10 mM magnesium acetate, 10 Units NgoMIV and 4 Units Avr II. The restriction digestion will create compatible ends for cloning the 4D5 MAb proprotein into pLSBC1767. The reaction was incubated at 37° C. for 2 hours and the 2.1 Kb fragment was gel isolated using the QIAquick Gel Extraction kit as described earlier. The recovery of the NgoMIV and Avr II digested fragment was verified by gel electrophoresis. The approximately 9.7 Kb NgoMIV and Avr II digested p LSBC1767 fragment was prepared similar to above and the 9.7 Kb fragment was verified by agarose gel electrophoresis. The 2.1 Kb NgoMIV and Avr II 4D5 MAb proprotein was cloned into the NgoMIV and Avr II prepared pLSBC1767 plasmid to create pLSBC1773 (Seq ID No: 93). A 50 μL ligation reaction containing 10 μL prepared 4D5 Mab proprotein, 15 μL pLSBC1767 vector, 800 Units T4 DNA Ligase, 50 mM Tris-HCl (pH 7.5), 10 mM MgCl₂, 25 μg/mL BSA, 10 mM DTT, 1 mM ATP was incubated at 14° C. overnight. The ligation was ethanol precipitated and used to transform electrocompetent JM109 as previously described. Cells were plated on LB plates containing 50 μg/mL ampicillin and grown overnight at 37° C. Individual colonies were picked and used to inoculate 1 mL Super Broth (SB) containing 500 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and plasmid eluted with 100 μL EB buffer. Clones were confirmed to contain the 2.1 Kb insert and the 9.7 Kb vector fragments by restriction enzyme mapping with NgoMIV and Avr II followed by agarose gel electrophoresis. The 4D5 MAb proprotein was sequenced using standard methods to verify the sequence.

Infectious transcripts were synthesized in-vitro from pLSBC1741 clones using the mMessage mMachine T7 kit (Ambion, Austin, Tex.) following the manufacturers directions. Briefly, a 5.5 μL reaction containing 1 μL 10×Reaction buffer, 2.5 ptL 2×NTP/CAP mix, 1 μL Enzyme mix and 3.5 μL plasmid was incubated at 37° C. for 2 hours. The synthesized transcripts were encapsidated in a 40 μL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 40 μL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate a 26 to 27 day post sow Nicotiana benthamiana expressing the TMV 30K movement protein driven by the CaMV 35S promoter and containing the NOS terminator as a transgene was made by standard transformation techniques. High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of MAb protein.

Interstitial fluid from infected leaves of each plant was harvested 6 days post inoculation and screened by western blot analysis. Systemically infected upper leaves from individual plants were harvested. The secreted protein fraction, or interstitial fluid (IF) was extracted and analyzed for presence of recombinant protein. The leaf tissue was placed in a GF/B 0.8 mL Unifilter (Whatman, Clifton, N.J.), covered with 20 mM Tris-HCl (pH 7.0) and subjected to 760 mmHg vacuum for 30 seconds. The vacuum is released and re-applied three times to completely infiltrate the tissue with buffer. The residual buffer is discarded and the tissue dried by centrifugation at 400 rpm in a plate centrifuge for 10 seconds. The IF fraction is recovered into a 96-well microplate by centrifugation for 10 minutes at 3K rpm in a plate centrifuge. The samples were subjected to western blot analysis to verify the presence of the 4D5 heavy chain and light chain polypeptides and run under reducing and nonreducing conditions to determine the presence of expected interchain disulfide bonding. 20 μL IF sample was adjusted to 1×tris-glycine sample dye with and without 10% 2-mercaptoethanol. 20 μL of each sample was loaded on two separate 10-20% Novex Tris glycine gel and subsequently transferred to Nitrocellulose membrane. The membranes were blocked overnight in PBST containing 2.5% powdered skim milk and 2.5% BSA. One membrane was probed with a 1:3000 dilution of Goat anti-mouse kappa-HRP labeled sera and the second membrane was probed with 1:3000 dilution of Goat anti-mouse IgG-HRP labeled sera (Southern Biotechnology, Birmingham, Ala.) for 1 hour at room temperature. The blots were washed three times in PBST and the labelled proteins detected with the ECL+plus Western Blotting Detection System (Amersham Biosciences, Buckinghamshire, England). The anti kappa sera detected an approximately 25 KDa proteins on the reduced sample and a approximately 150 KD band on the non-reduced indicating the presence of interchain disulfide bridges and an assemble 4D5 monoclonal antibody. The anti gamma sera detected an approximately 50 KDa proteins on the reduced sample and a approximately 150 KDa band on the non-reduced indicating the presence of interchain disulfide bridges and an assemble 4D5 monoclonal antibody. The presence of a disulfide linked 4D5 MAb heterodimer consisting of the gamma heavy chain and the kappa light chain was shown.

### EXAMPLE 15

Cloning of the Chimeric Mouse-human 9e10 FAB

Messenger RNA (mRNA) enriched for sequences containing long poly A tracts was isolated from total human spleen RNA (Clontech, Palo Alto, Calif.) using Dynabeads Oligo (dT)₂₅ (Dynal, Oslo, Norway). The RNA was pelleted by centrifugation at 15 K rpm, 4° C. for 15 minutes, the supernatant removed and 1 mL of 70% ethanol added. The sample was centrifuged at 15 K rpm, 4° C. for 15 minutes, the supernatant removed and the pellet resuspended in nuclease free water (Ambion, Austin, Tex.) at a concentration of 1 mg/mL. 4 μg of the above prepared total RNA was adjusted to 20 μL with nuclease free water and incubated at 65° C. for 2 minutes and immediately applied to 20 μL of magnetic beads in binding buffer (20 mM Tris-HCl (pH 7.5), 1.0 M LiCl, 2 mM EDTA). The RNA and bead mixture were incubated for 5 minutes with constant rotating. The supernatant containing unbound material was removed and the beads were washed with 100 μL washing buffer (10 mM Tris-HCl (pH 7.5), 0.15 M LiCl, 1 mM EDTA). Complementary DNA (cDNA) was synthesized in a 40 μL reaction containing 50 mM Tris HCl (pH 8.3), 75 mM KCl, 3 mM MgCl₂, 10 mM DTT, 2 Units RNasin (Promega, Madison, Wis.), 20 Units Superscript II (Invitrogen, Carlsbad, Calif.), 0.5 mM dATP, 0.5 mM dCTP, 0.5 mM dGTP, 0.5 mM dTTP, and the oligo dT bound RNA from above. The cDNA reaction was incubated at 42° C. for 60 minutes with constant rotation. The human heavy chain gamma constant region (C_{H}1 C_{H}2C_{H}3) was PCR amplified with upstream primer hCH15′sr (Seq ID No: 19), which anneals to the 5′ end of the gamma constant chain such that treatment with the 3′ to 5′ exonuclease activity of T4DNA polymerase will result in a “GC” 5′ extension where “G” is guanine and “C” is cytosine, and downstream primer hCH3avr3′ (Seq ID No: 20) which anneals to the 3′ end of the gamma constant chain and incorporates an Avr II site downstream of the termination codon for subsequent cloning. A 50 μL PCR reaction containing 0.4 μM hCH15′sr, 0.4 μM hCH3avr3′, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 2 μL prepared cDNA. The PCR reactions were amplified at 97° C. for 1 minute, 30 cycles of 94° C. for 30 seconds, 48° C. for 30 seconds, 72° C. for 45 seconds, and a 5 minute incubation at 72° C. The amplification of the desired approximately 1.0 Kb fragment was verified by agarose gel electrophoresis. The amplified human heavy chain constant domain was cloned into pCR4-TOPO (Invitrogen) following the manufacturers directions to create plasmid phCHTOPO (Seq ID No: 57). Briefly, 1 μL of PCR product, 1 μL vector, 1 μL of salt solution and 2 μL of water were mixed, incubated at room temperature for 5 minutes. The ligation was placed on ice and 25 μL of chemically competent Top 10 cells was added to the ligation and the mix was incubated on ice for 10 minutes. The transformation reaction was heat shocked by incubating at 42° C. for 30 seconds and immediately placed on ice and 250 μL of SOC was added. The transformation was allowed to recover by incubating at 37° C., 200 rpm shaking for 20 minutes. The transformation was plated out on LB plates containing ampicillin and grown overnight at 37° C. Individual colonies were used to inoculate 4.0 mL Luria Broth (LB) containing 100 μg/mL ampicillin in 14 mL culture tubes and grown overnight at 37° C. and 300 rpm. Plasmid was purified from turbid cultures using the QIAspin Miniprep kits (QIAGEN, Valencia, Calif.). Briefly, the cells were pelleted by centrifugation at 3 K rpm for 15 minutes in a plate centrifuge. The supernatant was drained from the cell pellets and the cells resuspended in 250 μL P1 Buffer by vortexing. 250 μL of P2 was added to the cells, mixed by inverting and incubated for 5 minutes to lyse the cells. 350 μL of N3 was added to the cell lysates, mixed by inverting and spun in centrifuge for 10 minutes at 15 K rpm. The supernatant was transferred to QIAspin column and spun in a centrifuge for 1 minute at 14 K rpm. The columns were washed with 0.75 mL of PB, followed by two washes with 0.75 mL of PE and dried. 100 μL EB buffer was added to the purified plasmid, incubated for 1 minute, and subsequently centrifuged for 1 minute at 15K rpm to elute the purified plasmid. The purified phCHTOPO plasmid was subjected to nucleic acid sequencing using standard methods to verify the human gamma IgGI heavy chain constant sequence.

The KP6 propeptide encoding sequence was PCR amplified from plasmid pLSBC1731 with upstream primer KP6v15′sr, which was designed to anneal to the 5′ end of the KP6 propeptide encoding sequence such that treatment with the 3′ to 5′ exonuclease activity of T4DNA polymerase will result in a “GCG” 5′ extension where “G” is guanine and “C” is cytosine, and downstream primer KP6v13′sr (Seq ID No: 24), which was designed to anneal to the 3′ end of the KP6 propeptide encoding sequence such that treatment with the 3′ to 5′ exonuclease activity of T4 DNA polymerase will result in a “CC” 5′ extension where “C” is cytosine. Alternately, the KP6 propeptide encoding sequence was PCR amplified from plasmid pLSBC1731 with upstream primer KP6v15′sr and downstream primer KP6v23′sr (Seq ID No: 15), which was designed to anneal to the 3′ end of the KP6 propeptide encoding sequence such that treatment with the 3′ to 5′ exonuclease activity of T4 DNA polymerase will result in a “CC” 5′ extension where “C” is cytosine. The human kappa light chain constant domain (C_{L}) sequence was PCR amplified from plasmid huscFabm1A6 (Seq ID No: 59) with upstream primer HuCL5′sr (Seq ID No: 21), which anneals to the 5′ end of the (C_{L}) domain such that treatment with the 3′ to 5′ exonuclease activity of T4DNA polymerase will result in a “CG” 5′ extension where “G” is guanine and “C” is cytosine, and downstream primer HuCL3′sr (Seq ID No: 22), which is designed to anneal to the 3′ end of the (C_{L}) domain such that treatment with the 3′ to 5′ exonuclease activity of T4 DNA polymerase will result in a “CGC” 5′ extension where “G” is guanine and “C” is cytosine. Alternately, the human kappa light chain constant domain (C_{L}) sequence was PCR amplified from plasmid—huscFabm1A6 (Seq ID No: 59) with upstream primer HuCL5′sr and downstream primer HuCLv23′sr (Seq ID No: 16), which is designed to anneal to the 3′ end of the (C_{L}) domain such that treatment with the 3′ to 5′ exonuclease activity of T4 DNA polymerase will result in a “CGC” 5′ extension where “G” is guanine and “C” is cytosine. Separate 50 μL PCR reactions containing 0.4 μM upstream primer, 0.4 downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 0.01 μL template plasmid. The PCR reactions were amplified at 97° C. for 1 minute, 25 cycles of 94° C. for 30 seconds, 55° C. for 15 seconds, 72° C. for 20 seconds, and 2 minutes at 72° C. The amplification of the desired approximately 120 bp KP6 propeptide encoding sequences and the 300 bp human kappa C_{L} sequences were confirmed by agarose gel electrophoresis.

The 9E10 light chain variable domain (V_{L}) was PCR amplified from plasmid pLSBC1736 with upstream primer 9E10Lngo5′ (Seq ID No: 10) which contains a Ngo MIV site compatible for cloning into vector pLSBC1767, which contains the alpha-amylase signal peptide, and downstream primer 9E10L3′sr (Seq ID No: 11), which is designed to anneal to the 3′ end of the (V_{L}) domain such that treatment with the 3′ to 5′ exonuclease activity of T4 DNA polymerase will result in a “GC” 5′ extension where “G” is guanine and “C” is cytosine. The Ngo MIV site joins the signal peptide to the FR1 of the 9E10 variable region of the light chain and directs the secretion of the artificial proprotein to the ER. The 9E10 heavy chain variable domain (V_{H}) was PCR amplified from the plasmid pLSBC1736 with upstream primer 9E10H5′srs (Seq ID No: 12), which anneals to the 5′ end of the C sequence such that treatment with the 3′ to 5′ exonuclease activity of T4 DNA polymerase will result in a “GG” 5′ extension where “G” is guanine, and downstream primer 9E10H3′sr (Seq ID No: 13) which anneals to the 3′ end of the V_{H} coding sequence such that treatment with the 3′ to 5′ exonuclease activity of T4DNA polymerase will result in a “CG” 5′ extension where “G” is guanine and “C” is cytosine.

The human heavy chain gamma constant region (C_{H}1C_{H}2C_{H}3) was PCR amplified from plasmid phCHTOPO with upstream primer hCHl5′sr and downstream primer hCH3avr3′. Separate 50 μL PCR reactions were set up to amplify the 9E10 V_{L}, 9E10 V_{H}c, and the phCHTOPO gamma constant domain containing 0.4 μM upstream primer, 0.4 μμM downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 0.01 μL template plasmid. The PCR reactions were amplified at 97° C. for 1 minute, 25 cycles of 94° C. for 30 seconds, 55° C. for 15 seconds, 72° C. for 20 seconds, and 2 minutes at 72 C. The amplification of the desired approximately 350 bp 9E10 V_{L} sequence, 380 bp 9E10 V_{H} sequence and 1.0 Kb human gamma constant sequence were confirmed by agarose gel electrophoresis.

The amplified KP6 propeptide encoding sequences, human kappa C_{L} sequences, 9E10 V_{L} sequence, 9E10 V_{H} sequence and the human gamma constant sequence were purified using the Strataprep PCR Purification Kit (Stratagene, La Jolla, Calif.) following manufacturers recommendations. Briefly, an equal volume of DNA-binding solution was added to the PCR product, mixed and transferred to the spin column. The column was centrifuged for 30 seconds at 14 K rpm. The column was washed two times with 750 μL of wash buffer and centrifuged for 30 seconds to dry. 50 μL elution buffer was added to the column and the PCR fragment eluted with by centrifugation at 14 K rpm for 30 seconds.

The purified PCR amplified fragments were ligated together in separate 20 μL ligation reactions. The first reaction contained 0.3 μL 9E10 V_{L} PCR fragment, 1 μL HuCL3′sr primed human kappa C_{L} PCR fragment, 1 μL KP6v13′sr primed KP6 propeptide PCR fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase and 1.2 Units T4 Polynucleotide Kinase. The second reaction contained 0.3 μL 9E10 V_{L} PCR fragment, 1 μL HuCLv23′sr primed human kappa C_{L} PCR fragment, 1 μL KP6v23′sr primed KP6 propeptide PCR fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase and 1.2 Units T4 Polynucleotide Kinase. The reactions were incubated at room temperature for 1 hour. The first reaction was PCR amplified with upstream primer 9E10Lngo5′ and downstream primer KP6vl3′sr and the second reaction was PCR amplified with upstream primer 9E10Lngo5′ and downstream primer KP6v23′sr in separate 50 μL PCR reaction 0.4 μM upstream primer, 0.4 μM downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 1 μL template plasmid. The PCR reactions were amplified at 97° C. for 1 minute, 25 cycles of 94° C. for 30 seconds, 55° C. for 15 seconds, 72° C. for 20 seconds, and a final step of 2 minutes at 72° C. The reactions were electrophoresed on a 1% agarose gel with TAE and 0.5 μg/mL ethidium bromide. The 800 bp PCR amplified 9E10 V_{L}-human C_{L}-KP6 fragments was cut from the gel and purified from the agarose slice using the MinElute gel extraction kit following the manufacturers instructions. Briefly, 3 volumes of QG buffer was added to each of the gel fragments, the mixture was incubated at 50° C. for 10 minutes with occasional agitation. A volume of isopropanol equal to the gel slice volume was added to the dissolved gel slice, mixed, applied to the column and centrifuged at 14K rpm for 1 minute. The column was washed with 500 μL Buffer QB followed by a wash with 750 μL PE and the purified fragment eluted in 10 μL EB. A separate 20 μL ligation reaction containing 1 μL 9E10 V_{H} PCR fragment, 1 μL human gamma heavy chain constant PCR fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase, 1.2 Units T4 Polynucleotide Kinase was incubated at room temperature for 1 hour. The ligation was electrophoresed on a 1% agarose gel with TAE and 0.5 μg/mL ethidium bromide. The 1.4 Kb ligated 9E10 V_{H}-human gamma constant fragment was cut from the gel and purified from the agarose slice using the MinElute gel extraction kit following the manufacturers instructions as describe previously and the purified fragment eluted in 10 μL EB.

The purified 9E10 V_{L}-human C_{L}-KP6 9E10Lngo5′-KP6v13′sr amplified fragment and the 9E10 V_{H}-human gamma constant fragment were ligated together in a 20 μL ligation reaction containing 7 μL 9E10 V_{L}-human C_{L}-KP6 9E10Lngo5′-KP6v13′sr amplified fragment, 6 μL 9E10 V_{H}-human gamma constant fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase, 1.2 Units T4 Polynucleotide Kinase. In a separate reaction, the purified 9E10 V_{L}-human C_{L}-KP6 9E10Lngo5′-KP6v23′sr amplified fragment and the 9E10 V_{H}-human gamma constant fragment were ligated together in a 20 μL reaction containing 7 μL 9E10 V_{L}-human C_{L}-KP6 9E10Lngo5′ and KP6v23′sr amplified fragment, 6 μL 9E10 V_{H}-human gamma constant fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase, 1.2 Units T4 Polynucleotide Kinase. The reactions were incubated at room temperature for 1 hour. The reactions were PCR amplified in separate 50 μL reactions with upstream primer 9E10Lngo5′ and downstream primer hCH3avr3′ containing 0.4 μM upstream primer, 0.4 μM downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 1 μL template plasmid. The PCR reactions were amplified at 97° C. for 1 minute, 15 cycles of 94° C. for 30 seconds, 55° C. for 15 seconds, 72° C. for 60 seconds, and a final step of 2 minutes at 72° C. The amplification of the desired approximately 2.1 Kb ligation products was confirmed by agarose gel electrophoresis. The PCR amplified products were purified using the Strataprep PCR Purification Kit (Stratgene) following manufacturers recommendations as described previously and eluted in 30 μL water. The PCR amplified product from the ligation of the 9E10 V_{L}-human C_{L}-KP6 9E10Lngo5′-KP6v13′sr amplified fragment and the 9E10 V_{H}-human gamma constant fragment was cloned into pCR4-TOPO (Invitrogen) following the manufacturers directions to create plasmid p9E10chimericv1-1 (Seq ID No: 61). In a separate reaction, the PCR amplified product from the ligation of the 9E10 V_{L}-human C_{L}-KP6 9E10Lngo5′-KP6v23′sr amplified fragment and the 9E10 V_{H}-human gamma constant fragment was cloned into pCR4-TOPO (Invitrogen) following the manufacturers directions to create plasmid p9E10chimericv2-1 (Seq ID No: 63). Briefly, 0.5 μL of PCR product, 1 μL vector, 1 μL of salt solution and 2.5 μL of water were mixed, incubated at room temperature for 5 minutes. The ligations were placed on ice and 25 μL of chemically competent Top 10 cells was added to the ligations and the mix was incubated on ice for 10 minutes. The transformation reaction was heat shocked by incubating at 42° C. for 30 seconds and immediately placed on ice and 250 μL of SOC was added. The transformation was allowed to recover by incubating at 37° C., 200 rpm shaking for 20 minutes. The transformation was plated out on LB plates containing ampicillin and grown overnight at 37° C. Individual colonies were used to inoculate 4.0 mL Luria Broth (LB) containing 100 μg/mL ampicillin in 14 mL culture tubes and grown overnight at 37° C. and 300 rpm. Plasmid was purified from turbid cultures using the QIAspin Miniprep kits (QIAGEN) as previously described and eluted in 50 μL EB buffer. The purified p9E10chimericv1-1 and p9E10chimericv2-1 plasmids was subjected to nucleic acid sequencing using standard methods.

The chimeric 9E10 V_{L}-human C_{L}-KP⁶- 9E10 V_{H} encoding sequences were PCR amplified from plasmid p9E10chimericv1-1 and p9E10chimericv2-1 in separate reactions with upstream primer 9E10Lngo5′ and downstream primer 9E1OH3′sr. The human heavy chain gamma constant region (C_{H}1C_{H}2C_{H}3) was PCR amplified from plasmid phCHTOPO with upstream primer hCH15′sr and downstream primer hCH3avr3′. Separate 50 μL PCR reactions containing 0.4 μM upstream primer, 0.4 μM downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 0.5 μL template plasmid. The PCR reactions were amplified at 97° C. for 1 minute, 15 cycles of 94° C. for 30 seconds, 55° C. for 15 seconds, 72° C. for 40 seconds, and a final step of 2 minutes at 72° C. The amplification of the desired approximately 1.1 Kb 9E10 V_{L}-human C_{L}-KP6-9E10 V_{H} encoding sequences and 1.0 Kb human gamma constant sequence were confirmed by agarose gel electrophoresis. The PCR amplified products were electrophoresed on a 1% agarose gel with TAE and 0.5 μg/mL ethidium bromide. The 1.1 Kb 9E10 V_{L}-human C_{L}-KP6-9E10 V_{H} encoding sequences and 1.0 Kb human gamma constant sequence were cut from the gel and purified from the agarose slice using the MinElute gel extraction kit following the manufacturers instructions as describe previously and the purified fragment eluted in 10 μL EB. The purified 1.1 Kb 9E10 V_{L}-human C_{L}-KP⁶- 9E10 V_{H} encoding sequences amplified from plasmid p9E10chimericv1-1 and 1.0 Kb human gamma constant sequence fragment were ligated together in a 20 μL ligation reaction containing 1.5 μL each fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase, 1.2 Units T4 Polynucleotide Kinase and incubated at room temperature for 2 hours. In a separate reaction, the purified 1.1 Kb 9E10 V_{L}-human C_{L}-KP6- 9E10 VH encoding sequences amplified from plasmid p9E10chimericv2-1 and 1.0 Kb human gamma constant sequence fragment were ligated together in a 20 μL ligation reaction containing 1.5 μL each fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase, 1.2 Units T4 Polynucleotide Kinase and incubated at room temperature for 2 hours. The reaction was incubated for 15 minutes at 75° C. to inactivate enzymes. The 20 μL ligation reactions were adjusted to 50 mM potassium acetate, 20 mM Tris-Acetate pH 7.9, 1 mM DTT, 10 mM magnesium acetate, and subsequently digested for 2 hours at 37° C. with 10 Units NgoMIV, 4 Units Avr II and 10 Units Dpn I. The restriction digestions will create compatible ends for cloning the 9E10 chimeric MAb proproteins into pLSBC1767. The reactions were gel isolated using the MinElute Gel Extraction kit as described earlier. The recovery of the NgoMIV and Avr II digested fragments was verified by gel electrophoresis. The approximately 2.1 Kb NgoMIV and Avr II digested fragment from pLSBC1767 was prepared similar to above and the 2.1 Kb fragment was verified by agarose gel electrophoresis.

The 2.1. Kb NgoMIV and Avr II 9E10 chimeric MAb proprotein derived from p9E10chimericv1-1 was cloned into the NgoMIV and Avr II prepared pLSBC1767 plasmid to create pLSBC2500. The 2.1 Kb NgoMIV and Avr II 9E10 chimeric MAb proprotein derived from p9E10chimericv2-1 was cloned into the NgoMIV and Avr II prepared pLSBC1767 plasmid to create pLSBC2502. Separate 30 μL ligation reactions containing 6 μL prepared insert, 2 μL pLSBC1767 vector, 800 Units T4 DNA Ligase, 50 mM Tris-HCl (pH 7.5), 10 mM MgCl₂, 25 μg/mL BSA, 10 mM DTT, 1 mM ATP were incubated at 14° C. overnight. Bacterial transformations into electro-competent JM109 cells was performed with a Gene Pulser electroporator (BioRad) as described previously. Plasmids were purified from turbid cultures using the QIAprep Spin Miniprep Kit (QIAGEN) as described previously and eluted with 50 μL Buffer EB. Clones were confirmed to contain the 2.1 Kb insert and the 9.7 Kb vector fragments by restriction enzyme mapping with NgoMIV and Avr II followed by agarose gel electrophoresis. The 9E10 chimeric MAb proprotein in pLSBC2500 and pLSBC2502 were sequenced using standard methods to verify the sequence.

Construction of pLSBC2505

The 9E10 chimeric Fab proprotein encoding sequence was PCR amplified from plasmid p9E10chimericv2-1 with upstream primer 9E10Lngo5′ and downstream primer hCHC2avr3′ (Seq ID No: 25), which anneals to the 3′ end of the C_{H}1 coding sequence and incorporates a termination codon followed by an Avr II site compatible for cloning into vector pLSBC1767. The 9E10 chimeric Fab proprotein encoding sequence from p9E10chimericv2-1 was PCR amplified in a 50 μL reactions containing 0.8 μM upstream primer, 0.8 μM downstream primer, 1×Expand High Fidelity Buffer with MgCl₂, 0.2 mM dATP, 0.2 mM dCTP, 0.2 mM dGTP, 0.2 mM dTTP, 3.5 Units Expand High Fidelity Polymerase and 0.05 μL template plasmid. The PCR reactions were amplified at 97° C. for 1 minute, 25 cycles of 94° C. for 30 seconds, 55° C. for 15 seconds, 72° C. for 30 seconds, and a final step of 2 minutes at 72° C. The amplification of the desired approximately 1.4 Kb 9E10 chimeric Fab proprotein encoding sequence was confirmed by agarose gel electrophoresis. The PCR amplified products were electrophoresed on a 1% agarose gel with TAE and 0.5 μg/mL ethidium bromide and the amplified 1.4 Kb 9E10 chimeric Fab proprotein encoding sequence was purified using the Strataprep PCR Purification Kit (Stratagene) following manufacturers recommendations as previously described. The prepared PCR amplified product was each digested in with NgoM IV and Avr II. A 20 uL reactions containing 3 μL prepared PCR fragment, 10 Units NgoM IV, 4 Units Avr II, 5mM potassium acetate, 20 mM Tris-acetate, 10 mM magnesium acetate, 1 mM DTT were incubated at 37° C. for 2 hours. The digested product was electrophoresed on a 1% agarose gel with TAE and 0.5 μg/mL ethidium bromide. The 1.4 Kb 9E10 chimeric Fab encoding sequence was cut from the gel and purified from the agarose slice using the MinElute gel extraction kit following the manufacturers instructions as describe previously and the purified fragment eluted in 10 μL EB.

The 1.4 Kb NgoMIV and Avr II PCR amplified 9E10 chimeric Fab proprotein amplified with 9E10Lngo5′ and hCHC2avr3′ primers derived from p9E10chimericv2-1 was cloned into the NgoMIV and Avr II prepared pLSBC1767 plasmid to create pLSBC2505. A 30 μL ligation reactions containing 4 μL prepared insert, 2 μl pLSBC1767 vector, 800 Units T4 DNA Ligase, 50 mM Tris-HCl (pH 7.5), 10 mM MgCl₂, 25 μg/mL BSA, 10 mM DTT, 1 mM ATP were incubated at 14° C. overnight. Bacterial transformations into electro-competent JM109 cells was performed with a Gene Pulser electroporator (BioRad) as described previously. Plasmids were purified from turbid cultures using the QIAprep Spin Miniprep Kit (QIAGEN) as described previously and eluted with 50 μL Buffer EB. Clones were confirmed to contain the 1.4 Kb insert and the 9.7 Kb vector fragments by restriction enzyme mapping with NgoMIV and Avr II followed by agarose gel electrophoresis. The 9E10 chimeric Fab proprotein in pLSBC2505 was sequenced using standard methods to verify the sequence.

### EXAMPLE 16

Cloning of FABS Containing Propeptide Sequence Variants and Expression Analysis

Construction of pLSBC2511 (Seq ID No: 65) and pLSBC2512 (Seq ID No: 67)

The 9E10 chimeric Fab proprotein encoding sequence was PCR amplified from plasmid pLSBC2500 with upstream primer 9E10Lngo5′ and downstream primer ch1Ctavr3′ (Seq ID No: 18), which anneals to the 3′ end of the C_{H}1 coding sequence and incorporates a termination codon followed by an Avr II site compatible for cloning into vector pLSBC1766 (Seq ID No: 89). Alternatively, the 9E10 chimeric Fab proprotein encoding sequence was PCR amplified from plasmid pLSBC2505 with upstream primer 9E10Lngo5′ and downstream primer ch1Ctavr3′. The 9E10 chimeric Fab proprotein encoding sequences from pLSBC2500 and pLSBC2505 were PCR amplified in separate reactions containing 0.8 μM upstream primer, 0.8 μM downstream primer, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase and 0.03 μL template plasmid. The PCR reactions were amplified at 97° C. for 1 minute, 15 cycles of 94° C. for 30 seconds, 55° C. for 30 seconds, 72° C. for 30 seconds, and a final step of 5 minutes at 72° C. The amplification of the desired approximately 1.4 Kb 9E10 chimeric Fab proprotein encoding sequences of pLSBC2500 and pLSBC2505 were confirmed by agarose gel electrophoresis. A phenol-chloroform extraction series and ethanol precipitation was performed on the PCR amplified products as previously described. The prepared pLSBC2500 and pLSBC2505 PCR amplified products were each digested in with NgoM IV and Avr II. Separate 25 uL reactions containing 10 μL prepared PCR fragment, 10 Units NgoM IV, 4 Units Avr II, 50mM potassium acetate, 20 mM Tris-acetate, 10 mM magnesium acetate, 1 mM DTT were incubated at 37° C. for 2 hours, and electrophoresed on a 1.0% agarose gel. The gel was stained with GelStar (Cambrex Bio Science) following the manufacturers directions. The approximately 1.4 Kb fragments were isolated from the agarose using QIAquick gel extraction kit following the manufacturers instructions. The recovery of the NgoM IV/Avr II digested fragments were verified by gel electrophoresis.

The 1.4 kb NgoM IV/Avr II prepared 9E10 chimeric Fab proprotein from pLSBC2500 was cloned into the NgoM IV/Avr II prepared pLSBC1766 plasmid to create pLSBC2511 (Seq ID No: 65). The 1.4 kb NgoM IV/Avr II prepared 9E10 chimeric Fab proprotein from pLSBC2505 was cloned into the NgoM IV/Avr II prepared pLSBC1766 plasmid to create pLSBC2512 (Seq ID No: 67). Separate 50 μL ligation reactions containing 10 μL NgoM IV/Avr II prepared 9E10 chimeric Fab proprotein insert, 0.4 μg NgoM IV/Avr II prepared pLSBC1766, 800 Units T4 DNA Ligase, 50 mM Tris-HCl (pH 7.5), 10 mM MgCl₂, 25 μg/mL BSA, 10 mM DTT, 1 mM ATP were incubated at 14° C. overnight. The ligation reactions were ethanol precipitated with 4 volumes ethanol and 0.67 volumes 5M NH₄Acetate, pelleted by centrifugation and washed with 70% ethanol. The washed pellets were resuspended in 6 μL 10 mM Tris-HCL (pH 8.0).

Construction of pLSBC2514 (Seq ID No: 69)

The KP6 propeptide encoding sequence was PCR amplified from plasmid pLSBC2500 with upstream primer KP6v15′sr (Seq ID No: 23) and downstream primer natKp6Ct 3′ (Seq ID No: 28) which was designed to anneal to the 3′ end of the KP6 propeptide encoding sequence such that treatment with the 3′ to 5′ exonuclease activity of T4DNA polymerase will result in a “GCC” 5′ extension where “G” is guanine and “C” is cytosine. The 9E10 chimeric light chain was PCR amplified from plasmid pLSBC2500 with upstream primer 9E10Lngo5′ and downstream primer NatKp6Nt3′ (Seq ID No: 26) which was designed to anneal to the 5′ end of the KP6 propeptide encoding sequence such that treatment with the 3′ to 5′ exonuclease activity of T4DNA polymerase will result in a “CGC” 5′ extension where “G” is guanine and “C” is cytosine. The NgoM IV site joins the signal peptide to the FR1 of the 9E10 variable light region and directs the secretion of the artificial proprotein to the ER. The 9E10 chimeric Fd heavy chain (V_{H}C_{H}1) was PCR amplified from the plasmid pLSBC2500 with downstream primer ch1Ctavr3′ and upstream primer NatKp6Ct5′ (Seq ID No: 27) which was designed to anneal to the 5′ end of the KP6 propeptide encoding sequence such that treatment with the 3′ to 5′ exonuclease activity of T4DNA polymerase will result in a “CGG” 5′ extension where “G” is guanine and “C” is cytosine. The KP6 propeptide encoding sequence, 9E10 chimeric heavy chain Fd sequence and 9E10 chimeric kappa light chain sequences were PCR amplified in separate 25 μL PCR reactions containing 0.8 μM upstream primer, 0.8 μM downstream, 1×Expand High Fidelity Buffer with MgCl₂, 0.2 mM dATP, 0.2 mM dCTP, 0.2 mM dGTP, 0.2 mM dTTP, 1.8 Units Expand High Fidelity Polymerase and 0.03 μL plasmid template. The PCR reactions were amplified at 95° C. for 2 minute, 15 cycles of 95° C. for 30 seconds, 55° C. for 30 seconds, 72° C. for 1 minute, and a final step of 7 minutes at 72° C. The amplification of the desired approximately 100 bp KP6 propeptide fragment, 700 bp 9E10 chimeric Fd fragment and 700 bp 9E10 chimeric light chain fragment were verified by agarose gel electrophoresis.

The PCR amplified KP6 propeptide encoding fragment, 9E10 chimeric heavy chain Fd fragment and 9E10 chimeric kappa light chain fragment were digested with Dpn I. 5 Units Dpn I was added to each PCR reaction and incubated at 37° C. for 1 hour followed by 80° C. for 20 minutes. The Dpn I digested PCR fragments were phenol-chloroform extracted followed by ethanol precipitation. The pellets were resuspended in 20 μL 10 mM Tris-HCL (pH 8.0).

The purified PCR amplified fragments were ligated together in a 20 μL ligation reaction. The reaction contained 18 ng KP6 propeptide fragment, 126 ng 9E10 chimeric Fd fragment, 126 ng 9E10 chimeric light fragment, 50 mM Tris-HCl (pH 7.5), 10 mM MgCl₂, 25 μg/mL BSA, 10 mM DTT, 0.2 mM dTTP, 0.2 mM dATP, 1 mM ATP, 0.6 Units T4 DNA Polymerase, 1.2 Units T4 DNA Ligase, and 1.2 Units T4 Polynucleotide Kinase. The reaction was incubated at 23° C. for 1.5 hours and then heat killed at 75° C. for 15 minutes. The ligation of the desired approximately 1.4 kb 9E10 chimeric Fab proprotein fragment was verified by agarose gel electrophoresis. A phenol chloroform extraction series was performed on the ligation product followed by ethanol precipitation. The pellet was digested in a 25 uL reaction containing 10 Units NgoM IV, 4 Units Avr II, 50 mM potassium acetate, 20 mM Tris-acetate, 10 mM magnesium acetate, 1 mM DTT. The digest was incubated at 37° C. for 2 hours, and electrophoresed on a 1.0% agarose gel to separate the approximately 1.4 Kb fragment. The gel was stained with GelStar (Cambrex Bio Science) and the approximately 1.4 Kb fragment was isolated. The fragment was purified away from the agarose using QIAquick gel extraction kit following the manufacturers instructions. The recovery of the NgoM IV/Avr II digested fragment was verified by gel electrophoresis. The prepared 1.4 kb NgoM IV/Avr II prepared 9E10 chimeric Fab proprotein from pLSBC2500 was cloned into the NgoM IV/Avr II prepared pLSBC1766 plasmid to create pLSBC2514 (Seq ID No: 69). A 50 μL ligation reaction containing 15 μL NgoM IV/Avr II prepared 9E10-Hum chimeric Fab fragment, 0.4 μg NgoM IV/Avr II prepared pLSBC1766, 800 Units T4 DNA Ligase, 50 mM Tris-HCl (pH 7.5), 10 mM MgCl₂, 25 μg/mL BSA, 10 mM DTT, 1 mM ATP were incubated at 14° C. overnight to create pLSBC2514. The ligation reaction was ethanol precipitated and the pellet was resuspended in 6 μL 10 mM Tris-HCL (pH 8.0).

pLSBC2511, pLSBC2512, and pLSBC2514

The ligations of pLSBC2511, pLSBC2512, and pLSBC2514 were used in separate reactions to transform electro-competent JM109 cells was performed with a Gene Pulser electroporator (BioRad) as described previously. Individual colonies were picked and used to inoculate 4 mL LB containing 200 μg/mL Carbenicillin in 14 mL tubes and grown overnight at 30° C. and 300 rpm. Plasmid was purified from turbid cultures using the QIAprep Spin Miniprep Kit (QIAGEN) as described previously and eluted with 50 μL Buffer EB. Clones were confirmed to contain the 1.4 kb insert and the 9.7 kb vector fragments by restriction enzyme mapping with NgoM IV and Avr II followed by agarose gel electrophoresis. The 9E10 chimeric Fab proproteins were sequenced using standard methods to verify the sequence.

Infectious transcripts were synthesized in-vitro from pLSBC2511, pLSBC2512, and pLSBC2514 clones using the mMessage mMachine T7 kit (Ambion, Austin, Tex.) following the manufacturers directions. Briefly, a 20 μL reaction containing 2 μL 10×Reaction buffer, 10 μL 2×NTP/CAP mix, 2×L Enzyme mix and 4×L plasmid was incubated at 37° C. for 1 hour. The synthesized transcripts were encapsidated in a 200 μL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 200 μL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate four 22 day post sow Nicotiana benthamiana expressing the TMV 30K movement protein driven by the CaMV 35S promoter and containing the NOS terminator as a transgene was made by standard transformation techniques. High levels of subgenomic RNA species were synthesized in virus-infected plant cells (Kumagai, MH. et al. (1993) Proc. Natl. Acad. Sci. USA 90:427-430), and serve as templates for the translation and subsequent accumulation of Fab protein.

Interstitial fluid from infected leaves of each plant was harvested 7 days post inoculation and screened by Coomassie stained protein gels. Systemically infected upper leaves from each of the four individual plants were harvested. The secreted protein fraction, or interstitial fluid (IF) was extracted and analyzed for presence of recombinant protein. The leaf tissue was placed in a GF/B 0.8 mL Unifilter (Whatman, Clifton, N.J.), covered with 20 mM Tris-HCl (pH 7.0) and subjected to 760 mmHg vacuum for 30 seconds. The vacuum is released and re-applied three times to completely infiltrate the tissue with buffer. The residual buffer is discarded and the tissue dried by centrifugation at 400 rpm in a plate centrifuge for 1 minute. The IF fraction was recovered in a 96-well microplate by centrifugation for 10 minutes at 3K rpm in a plate centrifuge. 20 μL of each IF sample was prepared for SDS-PAGE analysis by the addition of 5 μL 5×tris-glycine sample dye containing 10% 2-mercaptoethanol, for reducing gels, and then boiled for 2 minutes. Samples were separated on a 10-20% gradient Criterion gel (Bio-Rad) and the proteins were detected by Coomassie R-250 Brilliant blue staining. Protein banding in the reducing gel at approximately 25 KDa and 27 KDa indicates the presence of the desired 25 KDa heavy chain Fd and the 27 KDa light chain.

### EXAMPLE 17

Preproprotein Expression of 9E10 FAb in Plant Cells by Agroinfiltration

A FAb construct of 9E10 from pLSBC1736 is introduced into a T-DNA vector derived from pBI121 (Jefferson, R. A. et al., EMBO J 6 (1987) 3901-3907) using PacI and AvrII restriction enzymes wherein the GUS gene is replaced by the FAb sequence such that expression is driven by the 35S promoter. The T-DNA construct is transformed into Agrobacterium strain C58C1 carrying pCH32 (Hamilton, C. M., et al., Proc Natl Acad Sci U S A 93 (1996) 9975-9) by electroporation. The Agrobacterium is grown into a culture and used to agroinfiltrate (Scofield, S. R. et al., Science 274 (1996) 2063-5, Tang, X. et al., Science 274 (1996) 2060-3, Bendahmane, A., et. al, Plant Cell 11 (1999) 781-791) leaves of Nicotiana benthamiana. After two days proteins are extracted from the leaves and the resulting extracts are analyzed, for instance, by SDS-PAGE and Western blot or by reverse phase HPLC analysis to analyze the expression of the desired gene product.

### EXAMPLE 18

Preproprotein Expression of 9E10 FAb in Plant Cells in Transgenic Plants

The Agrobacterium strain carrying the T-DNA construct from Example 17 is used to transform leaf disks of Nicotiana tabacum, and transgenic plants are regenerated (Horsch, R. B., et al., Science 227 (1985) 1229-1231). Leaves from the transgenic plants are extracted to yield the FAb. The resulting extracts are analyzed, for instance, by SDS-PAGE and Western blot or by reverse phase HPLC analysis to analyze the expression of the desired gene product.

### Example 19

Preproprotein Expression of 4D5 MONOCLONAL ANTIBODY in Plant Cells by Agroinfiltration.

A MAb construct of 4D5 from pLSBC1773 is introduced into a T-DNA vector derived from pBI121 (Jefferson, R. A. et al., EMBO J 6 (1987) 3901-3907) using PacI and AvrII restriction enzymes wherein the GUS gene is replaced by the FAb sequence such that expression is driven by the 35S promoter. The T-DNA construct is transformed into Agrobacterium strain C58C1 carrying pCH32 (Hamilton, C. M., et al., Proc Natl Acad Sci U S A 93 (1996) 9975-9) by electroporation. The Agrobacterium is grown into a culture and used to agroinfiltrate (Scofield, S. R. et al., Science 274 (1996) 2063-5, Tang, X. et al., Science 274 (1996) 2060-3, Bendahmane, A., et. al, Plant Cell 11 (1999) 781-791) leaves of Nicotiana benthamiana. After two days proteins are extracted from the leaves and the resulting extracts are analyzed, for instance, by SDS-PAGE and Western blot or by reverse phase HPLC analysis to analyze the expression of the desired gene product.

### Example 20

Pre-proprotein Expression of 4D5 MONOCLONAL ANTIBODY in Plant Cells in Transgenic Plants

The Agrobacterium strain carrying the T-DNA construct from Example 19 is used to transform leaf disks of Nicotiana tabacum, and transgenic plants are regenerated (Horsch, R. B., et al., Science 227 (1985) 1229-1231). Leaves from the transgenic plants are extracted to yield the FAb. The resulting extracts are analyzed, for instance, by SDS-PAGE and Western blot or by reverse phase HPLC analysis to analyze the expression of the desired gene product.

### EXAMPLE 21

Pre-proprotein Expression of 4D5 MAb Transformed CHO Cells

The vector pC4 is used for the expression of 4D5 MAb pre-proprotein. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., J Biol. Chem. 253:1357-1370 (1978), Hamlin, J. L. and Ma, C., Biochem. et Biophys. Acta, 1097:107-143 (1990), Page, M. J. and Sydenham, M. A., Biotechnology 9:64-68) (1991). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen et al., Molec. Cell. Biol. 5:438-447 (1985)) plus a fragment isolated from the enhancer of the immediate early gene of human cytomegalovirus (CMV) (Boshart et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat insulin gene. Other high efficiency promoters can also be used for the expression, e.g., the human beta.-actin promoter, the SV40 early or late promoters or the long terminal repeats from other retroviruses, e.g., HIV and HTLVI. Clontech's Tet-Off and Tet-On gene expression systems and similar systems can be used to express the 4D5 MAb pre-proprotein in a regulated way in mammalian cells (Gossen, M., & Bujard, H., Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes BamHI and Asp718I and then dephosphorylated using calf intestinal phosphatase by procedures known in the art. The vector is then isolated from a 1% agarose gel.

The DNA sequence encoding the complete 4D5 MAb pre-proprotein gene including its leader sequence is amplified using PCR oligonucleotide primers corresponding to the 5′ and 3′ sequences of the gene. The 5′ primer has a sequence containing the BamHI restriction enzyme site followed by an efficient signal for initiation of translation in eukaryotes, as described by Kozak, M., J. Mol. Biol. 196:947-950 (1987), and 17 bases of the sequence of 4D5 MAb pre-proprotein. The 3′ primer has a sequence containing the Asp718I restriction site followed by nucleotides complementary to the 3′ terminus of the 4D5 MAb pre-proprotein gene.

The amplified fragment is digested with the endonucleases BamHI and Asp718I and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. 5 .mu.g of the expression plasmid pC4 is cotransfected with 0.5 .mu.g of the plasmid pSV2-neo using lipofectin (Felgner et al., Proc. Natl. Acad. Sci. USA 84:7413-7417 (1987)). The plasmid pSV2neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mu.M, 2 .mu.M, 5 .mu.M, 10 .mu.M, 20 .mu.M). The same procedure is repeated until clones are obtained which grow at a concentration of 100-200 mu.M. Expression of the desired gene product is analyzed, for instance, by SDS-PAGE and Western blot or by reverse phase HPLC analysis.

| Cell Name | Animal | Tissue |
|---|---|---|
| FBHE | bovine | heart |
| V79 379A | hamster, Chinese | lung |
| CHO-K1 | hamster, Chinese | ovary |
| NAGL-1 | human | B cells |
| MG-63 | human | bone |
| FS-1 | human | bone marrow stroma |
| SK-MG-1 | human | brain |
| WiDr | human | colon |
| A431 | human | epidermoid |
| Alexander cells | human | liver |
| WI-38 | human | lung |
| GAK | human | lymph node |
| Namalwa | human | lymphoblastoid |
| RMUG-S | human | ovary |
| RPMI 1788 | human | peripheral blood |
| NB-1 | human | sympatho-adrenal cell |
| HUV-EC-C | human | umbilical cord, vein |
| HeLa S3 | human | uterine cervix |
| SKN | human | uterus |
| 4G12 hybridoma | human-mouse | hybridoma, lymphoid x |
| | hybrid | myeloma |
| VERO 76 | monkey, African | kidney |
| | green | |
| COS-7 | monkey, African | kidney |
| | green | |
| C6/36 | mosquito | hatched larvae |
| MBT2 | mouse | bladder |
| AP-16 | mouse | brain, astrocyte-progenitor cell |
| MA-89 | mouse | brain, cerebra |
| Balb/c 3T3 A31-I-1 | mouse | embryo, whole |
| TLR3 | mouse | liver |
| WEHI-3b | mouse | myelomonocyte |
| DBC1.2 | mouse | nasal septum |
| Neuro-2aTG | mouse | region of spinal cord |
| MSS62 | mouse | spleen |
| EHS | mouse | spontaneous tumor |
| SIRC | rabbit | cornea |
| PC-12TG | rat | adrenal medulla |
| RBL-1 | rat | blood |
| RNB | rat | brain |
| F2408-No. 7 | rat | embryonic fibroblast |
| GH1 | rat | pituitary gland |
| L6 | rat | skeletal myoblast |
| 6-23 clone 6 | rat | thyroid, C cell |

### EXAMPLE 22

Optimization, Screening and Production of Antibodies in Plants

The affinity or activity of an antibody or antibody fragment (Fab) are modified to improve desired characteristics such as affinity as demonstrated in Carter, et al, (1992)Proc. Nat. Acad. Sci. vol. 89 (4285-4289). Once an antibody, whether native, chimeric or humanized with CDR exchanges, is obtained, positions in the variable heavy and light chain genes are identified as influencing the structure and function or binding of the antibody through molecular modeling comparisons of predicted structure and known crystal structures.

The identified or presumed influential positions are randomized to contain preferred amino acids for optimal structural organization as well as preferred non-immunogenic human sequences. Using DNA shuffling, multiple influential positions containing varied amino acids residues at any one position, are re-assorted to create a population of sequences which contain all combinations or many combinations of amino acids at these influential sites.

The population of antibody sequences created by DNA shuffling are cloned as described in EXAMPLE 2 to create a population preproprotein sequences which are cloned into GENEWARE expression vectors using restriction independent cohesive end cloning.

A series of computer controlled robots, data based tracking and information management systems are used to pick colonies, prepare plasmid clones, sequence, transcribe and encapsidated infectious transcripts in a high through-put (HTP) process. The encapsidated transcripts are used to infect plants which are subsequently harvested and extracted in a HTP manner such as leaf punches followed by HTP IF extraction or tissue homogenization.

The extracts are assayed in a HTP manner for a preferred activity such as antigen bind as determined by ELISA or other suitable assay. Additionally, it is preferred if the activity assay has a quantitative aspect. The samples are furthered evaluated to determine the quantity of the antibody present. This can be done with an ELISA to detect total antibody or with other suitable assays.

Identified targets can be immediately used to inoculate larger quantities of plants to obtain purified the antibody for further characterization, pre-clinical evaluation, and process development.

Concurrently, the expression system is scaled up to produce sufficiently large scale quantities for manufacturing. This may involve the creation of a plant line stably transformed with the preferred proprotein or antibody encoding genes. Plasmid, virus and seed are generated in large scale to accommodate the needs of the manufacturing process.

### EXAMPLE 23

Cloning and Expression Analysis of Follicle Stimulating Hormone Proprotein

Human follicle-stimulating hormone is a disulfide linked, heterodimeric protein containing the glycoprotein hormones alpha subunit and the follicle stimulating hormone beta subunit. The follicle stimulating hormone beta subunit was assembled from overlapping synthetic oligonucleotides in a 50 μL PCR reaction containing 0.1 μM KP509 (Seq ID No: 98), 0.1 μM KP510 (Seq ID No: 99), 0.1 μM KP511 (Seq ID No: 100), 0.1 82 M KP512 (Seq ID No: 101), 0.1 μM KP513 (Seq ID No: 102), 0.1 μM KP514 (Seq ID No: 103), 0.1 μM KP517 (Seq ID No: 106), 0.1 μM KP518 107, 0.1 μM KP519 (Seq ID No: 108), 0.1 μM KP520 (Seq ID No: 109), 0.1 μM KP521 (Seq ID No: 110), 1×ThermalAce Buffer, 0.2 mM dATP, 0.2 mM dCTP, 0.2 mM dGTP, 0.2 mM dTTP, 3.5 Units ThermalAce DNA Polymerase (Invitrogen) was amplified at 98° C. for 3 minutes, 20 cycles of 95° C. for 30 seconds, 50° C. for 30 seconds, 74° C. for 30 seconds and a final step of 74° C. for 5 minutes. The above PCR product was re-amplified in a 50 μL PCR reaction containing 0.5 μM KP515 (Seq ID No: 104), 0.5 μM KP522 (Seq ID No: 111), 1 μL PCR product, 1×Pfu Buffer, 1 mM dATP, 1 mM dCTP, 1 mM dGTP, 1 mM dTTP, 3.5 Units Pfu DNA Polymerase (Stratgene) was amplified at 98° C. for 3 minutes, 20 cycles of 95° C. for 30 seconds, 50° C. for 30 seconds, 74° C. for 30 seconds and a final step of 74° C. for 7 minutes. The PCR reaction was purified using the MinElute PCR purification kit (Qiagen) following the manufacturers instructions. The PCR fragment from the above reaction was cloned into pCRIIBlunt-TOPO (Invitrogen) following the manufacturers directions to create plasmid pLSB2622. The glycoprotein hormones alpha subunit was PCR amplified from a human cDNA clone derived from human mRNA. A 50 μL PCR reaction containing 0.5 μM KP516 (Seq ID No: 105), 0.5 μM KP523 (Seq ID No: 112), 0.3 μL plasmid template, 1×Pfu Buffer, 0.2 mM dATP, 0.2 mM dCTP, 0.2 mM dGTP, 0.2 mM dTTP, 3.5 Units Pfu Ultra DNA Polymerase (Stratgene) was amplified at 94° C. for 2 minutes, 25 cycles of 94° C. for 30 seconds, 55° C. for 30 seconds, 72° C. for 30 seconds and a final step of 72° C. for 7 minutes. The PCR reaction was purified using the MinElute PCR purification kit (Qiagen) following the manufacturers instructions. The PCR fragment from the above reaction was cloned into pCRIIBlunt-TOPO (Invitrogen) following the manufacturers directions to create plasmid pLSB2620. The pLSB2622 and pLSB2620 ligations were used to transform chemically competent Top 10 cells following the manufacturers directions. The transformations were plated out on LB plates containing antibiotic and grown overnight at 37° C. Individual colonies were used to inoculate 1.0 mL Super Broth (SB) containing antibiotic in 96 well 2.0 mL flat-bottom blocks and grown overnight at 37° C. and 400 rpm. Plasmid was purified from turbid cultures using the QIAprep 96 Turbo Miniprep kits (QIAGEN) as previously described. The purified pLSB2622 and pLSB2620 plasmids were subjected to nucleic acid sequencing using standard methods.

To assemble the follicle stimulating hormone proprotein encoding sequence, the beta subunit from clone pLSB2622 was amplified with upstream primer KP515 which anneals to the 5′ end of the beta subunit mature protein and contains a Ngo MIV site compatible for cloning into vector (pLSBC1767), and KP552 downstream primer anneals to the 3′ end of the beta subunit, removes the termination codon and fuses the subunit in frame to the 5′ end of the KP6 propeptide coding sequence. A 50 μL PCR reaction containing 0.5 μM KP515, 0.5 μM KP552, 0.2 μL plasmid template, 1×Pfu Buffer, 0.2 mM dATP, 0.2 mM dCTP, 0.2 mM dGTP, 0.2 mM dTTP, 3.5 Units Pfu Ultra DNA Polymerase (Stratgene) was amplified at 98° C. for 3 minutes, 20 cycles of 95° C. for 30 seconds, 55° C. for 30 seconds, 74° C. for 30 seconds and a final step of 74° C. for 7 minutes. The PCR reaction was purified using the MinElute PCR purification kit (Qiagen) following the manufacturers instructions. The glycoprotein hormones alpha subunit was amplified with from plasmid pLSB2620 was amplified with upstream primer KP551 which anneals to the 5′ end of the alpha subunit and fuses it in frame to the 3′ end of the KP6 propeptide coding sequence and KP523 downstream primer which anneals to the 3′ end of the alpha subunit including a translational termination codon followed by an Avr II site for subsequent cloning. A 50 μL PCR reaction containing 0.5 μM KP551, 0.5 μM KP523, 0.3 μL plasmid template, 1×ThermalAce Buffer, 0.2 mM dATP, 0.2 mM dCTP, 0.2 mM dGTP, 0.2 mM dTTP, 3.5 Units ThermalAce DNA Polymerase (Invitrogen) was amplified at 94° C. for 2 minutes, 25 cycles of 94° C. for 30 seconds, 55° C. for 30 seconds, 72° C. for 30 seconds and a final step of 72° C. for 7 minutes. The PCR reaction was purified using the MinElute PCR purification kit (Qiagen) following the manufacturers instructions. The above amplified fragments from pLSB2620 and pLSB2622 were fused by sequence overlap extension (SOE). A 50 μL PCR reaction containing 0.5 μM KP515, 0.5 μM KP523, 0.1 μL pLSB2620 PCR product, 0.1 μL pLSB2622 PCR product, 1×ThermalAce Buffer, 0.2 mM dATP, 0.2 mM dCTP, 0.2 mM dGTP, 0.2 mM dTTP, 3.5 Units ThermalAce DNA Polymerase (Invitrogen) was amplified at 94° C. for 2 minutes, 25 cycles of 94° C. for 30 seconds, 60° C. for 30 seconds, 72° C. for 30 seconds and a final step of 72° C. for 7 minutes. The PCR reaction was purified using the MinElute PCR purification kit (Qiagen) following the manufacturers instructions. A 50 μL reaction containing 10 μL purified PCR product, 50 mM potassium acetate, 20 mM Tris-Acetate pH 7.9, 1 mM DTT, 10 mM magnesium acetate, 10 Units NgoMIV and 4 Units Avr II was incubated at 37° C. for 3 hours and reaction was purified using the MinElute PCR purification kit (Qiagen) following the manufacturers instructions. The 0.7 Kb NgoMIV and Avr II digested follicle stimulating hormone proprotein encoding sequence was ligated into pLSBC1767 to create pLSB2634 (Seq ID No: 96). A 21 μL ligation reaction containing 50 ng NgoMIV and AvrII prepared pLSBC1767, 0.2 μL purified NgoMIV and Avr II digested PCR fragment, 1×Quick Ligation Buffer (New England Biolabs) and 1 μL Quick T4 DNA Ligase (New England Biolabs) was incubated at 25° C. fro 5 minutes. Bacterial transformations with DH5α competent cells (Invitrogen) were performed according to manufacturer recommendations. Cells were plated on LB plates containing 100 μg/mL ampicillin and grown overnight at 37° C. Individual colonies were picked and used to inoculate 1 mL Super Broth (SB) containing 800 μg/mL ampicillin in 96 well 2.0 mL flat-bottom blocks and grown overnight at 37° C. and 400 rpm. Plasmid was purified from turbid cultures using the QIAprep 96 Turbo Miniprep kits (QIAGEN, Valencia, Calif.) as previously described and eluted in 100 μL EB Buffer. pLSB2634 (Seq ID No: 96) clones were confirmed to contain a 0.7 Kb fragment by sequencing using standard methods.

Infectious transcript was synthesized in-vitro from pLSB2634 using the mMessage mMachine T7 kit (Ambion, Austin, Tex.) following the manufacturers directions. Briefly, a 10 μL reaction for each plasmid containing 1 μL 10×Reaction buffer, 5 μL 2×NTP/CAP mix, 1 μL Enzyme mix and 0.5 μg plasmid was incubated at 37° C. for 2 hours. The synthesized transcripts were encapsidated in a 50 μL reaction containing 0.1 M Na₂HPO₄-NaH₂PO₄ (pH 7.0), 0.5 mg/mL purified U1 coat protein (LSBC, Vacaville, Calif.) which was incubated overnight at room temperature. 0.1 mL of FES (0.1 M Glycine, 60 mM K₂HPO₄, 22 mM Na₂P₂O₇, 10 g/L Bentonite, 10 g/L Celite 545) was added to each encapsidated transcript. The encapsidated transcript from an each individual clone was used to inoculate 23 day post sow Nicotiana benthamiana. High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of follicle stimulating hormone protein.

Interstitial fluid from infected leaves of each plant was harvested 8 days post inoculation. Systemically infected upper leaves from each of the infected plants was harvested. The secreted protein fraction, or interstitial fluid (IF) was extracted and analyzed for presence of recombinant protein. The leaf tissue was covered with 50 mM Acetate (pH 5.0), 400 mM NaCl, 0.04% sodium metabisulfite and subjected to 760 mmHg vacuum for 2 minutes. The vacuum is released and re-applied three times to completely infiltrate the tissue with buffer. The IF fraction was recovered by centrifugation for 20 minutes at 4K rpm.

10 μL of each IF sample was prepared for SDS-PAGE analysis by the addition of 5 μL 5×tris-glycine sample dye containing 10% 2-mercaptoethanol and the mixture was boiled for 2 minutes. Samples were separated on a 10-20% Criterion gel (Bio-Rad) and the proteins were transferred to Nitrocellulose membrane for Western blot. The membranes were blocked overnight in TBST containing 2.5% powdered skim milk and 2.5% BSA. The membrane was probed with a 1:2000 dilution of Rabbit anti-human follicle stimulating hormone polyclonal sera (US Biologicals) for 1 hour at room temperature. The blots were washed three times in TBST and probed with a 1:2000 dilution of goat anti-rabbit-HRP labeled polyclonal sera for 1 hour at room temperature. The blots were washed three times in TBST and the labeled proteins detected with the ECL+plus Western Blotting Detection System (Amersham Biosciences, Buckinghamshire, England). The anti-follicle stimulating hormone sera detected an approximately 17 KDa beta protein and a 15 KDa alpha protein indicating that both the alpha and beta subunits were expressed, processed and secreted.

### EXAMPLE 24

Cloning and Expression Analysis of IL-12 Proprotein

IL-12 is a disulfide linked heterdimeric protein, composed of a 35 KDa subunit (p35) and a 40 KDa subunit (p40), and enhances the cytotoxicity of NK cells, induces PBL's to produce interferon gamma and stimulates the proliferation of PBL's. (Wolf et. al., J. of Immunol. (1991) 146(9):3074-81) The construction of an IL-12 proprotein expressing assembly is performed essentially as described in example 4. The IL-12 p35 subunit is PCR amplified from a cDNA clone with an upstream primer containing a NgoMIV site in frame with the mature protein coding sequence suitable for cloning in frame with the alpha amylase signal peptide of pLSBC1767 and downstream primer which removes the translational termination codon of p35 and fuses the 3′ end of the p35 sequence to the 5′ end of the KP6 propeptide sequence amplified from pLSBC1731. The IL-12 p40 subunit is PCR amplified from a cDNA clone with an upstream primer which fuses the 3′ end of the KP6 propeptide encoding sequence in frame with the 5′ end of the mature p40 coding sequence and downstream primer which anneals to the 3′ end of the p40 coding sequence and introduces an Avr II site following the translational termination codon suitable for cloning into pLSBC1767. The PCR amplified p35 subunit, KP6 propeptide encoding sequence of pLSBC1731 and the p40 subunit are assembled together to create the IL-12 proprotein coding sequence by sequence overlap extension (SOE). The resulting fragment is restriction enzyme digested and cloned into prepared pLSBC1767 vector. The ligation is used to transform competent E. coli cells and tranformants grown and plasmid DNA purified using standard techniques. The resultant IL-12 proprotein assembly in the viral vector is used to synthesize infectious transcript in-vitro using the mMessage mMachine T7 kit (Ambion, Austin, Tex.) following the manufacturers directions. The synthesized transcripts are encapsidated with purified U1 coat protein (LSBC, Vacaville, Calif.) and mixed with FES. The encapsidated transcript from an each individual clone was used to inoculate Nicotiana benthamiana. High levels of subgenomic RNA species were synthesized in virus-infected plant cells (Kumagai, M H. et al. (1993) Proc. Natl. Acad. Sci. USA 90:427-430), and serve as templates for the translation and subsequent accumulation of IL-12 protein.

Infected plant tissue is harvested and proteins are extracted and the resulting extracts are analyzed, for instance, by SDS-PAGE and Western blot or by reverse phase HPLC analysis to analyze the expression of the IL-12 gene product.

### EXAMPLE 25

Monoclonal Antibodies and Fabs In Patient-Specific Immunotherapy

The use of monoclonal antibodies (MAb) and polyclonal antibodies in the treatment of cancer and infectious disease is well established. These products exert their beneficial effects by binding to specific targets on the surface of malignant or pathogen cells, to mark these pathogenic cells for immune recognition and destruction. In addition to binding to targets, the constant region of antibodies can also serve effector functions that help modulate the type, magnitude and duration of the immune response.

Antibodies can also be fused, either at the gene level or post-translationally, to additional molecules such as toxins or radioisotopes, with the goal of increasing the therapeutic action against the unwanted cell. Such bifunctional immunotherapeutics thus consist of a targeting moiety provided by the antibody and a toxic payload provided by the toxin, enzyme, or radioisotope, which may play the major role in destroying the unwanted target cell.

In some applications it is desirable not to use a whole antibody molecule. The penetration of the immunoprotein through fine capillary beds and tissues on its way to finding and binding a target may best be achieved if the antibody is a fragment or subunit of the naturally produced native protein. Antibody fragments in this category include Fab, scFv, diabodies, tetrabodies, etc, each having a different conformation and binding functionality.

Nearly all antibodies and antibody fragments used in biomedical therapy are designed to bind to a common target on the pathogen or target cell. Upon administration, the antibodies home in a common cellular marker on a population of cells. Selectivity to a disease, and therapeutic index, in these applications is thus determined in large part by the protein or structure on the target cell against which the antibody was selected to bind. A product such as rituximab (Rituxan®), for example, will target all cells exposing the cellular marker CD20 on their surface; in this case, B cells of the immune system. The product can delete all B cells by targeting that common marker. The product is used to control B-cell non-Hodgkin's lymphoma (NHL), and rituximab works well by getting rid of malignant as well as non-malignant B cells from the patient. Because B-cell NHL is a clonal disease, while the patient's malignant B-cell clone is temporarily controlled, the healthy B cell arm of the patient's immune system is also destroyed as a consequence, leaving the patient temporarily immunocompromized until his bone marrow can generate new B cells.

Immunoproteins can also be used to target individual target proteins or structures on the surface of only some subpopulation on target cells. For example, if an antibody could be made against a tumor-specific marker on a malignant cell, that cell population would be targeted and the healthy cells of the same lineage spared. This is an example of highly selective immunotherapy compared to the example for rituximab, in which a panreactive cell-type antigen is targeted. Tumor- or pathogen-specific antibodies can be full-size MAb or polyclonal antibodies, or antibody fragments such as scFv, Fab, and other compositions described in the art.

One specific example of targeted immunotherapy is patient-specific immunotherapy, where the drug used is so selective as to work on only a single patient. To use the NHL example, an antibody or antibody fragment can be selected to target a marker on a clonal tumor, such as NHL. All B cells project an immunoglobulin molecule on their surface, such as IgM. Because each B-cell line, or clone, produces a unique antibody, the antibody sequence can be used as a tumor-specific marker of a malignant B-cell clone in NHL. An antibody, or antibody fragment, targeted to bind to the unique immunoglobulin sequence on the malignant B-cell tumor's surface can be expected to bind to, and help destroy, only the malignant clone of B cells while ignoring all other B-cell clones and thus sparing the healthy B cell arm of the patient's immune system. Such selectivity would have obvious advantages over the wholesale deletion of the B-cell arm of the immune system, such as is observed with rituximab, as no or minimal humoral immunosuppression would be expected. Because each tumor-specific marker is individual to that patient's specific B cell, the therapeutic antibody to be administered would be expected to show efficacy only in that patient and thus this therapy is considered patient-specific immunotherapy.

A patient's B-cell, NHL biopsy would be obtained and the exposed IgM (or any other tumor-specific antigen) is used to generate either a full-size antibody or a fragment of an antibody binding specifically to that antigen. The generation of a high-affinity antibody or antibody fragment can be achieved by methods known to those skilled in the art, and include immunization of an animal, panning of phage-display libraries, and the like. For human therapy, it would be preferable to use either a fully human antibody or antibody fragment, or a humanized animal-derived antibody or antibody fragment, to prevent potential concerns over immunogenicity with long-term use of the product.

An artificial open reading frame encoding the antibody or antibody fragment can be constructed, and the antibody or antibody fragment can be made and isolated by the methods shown in previous examples. The antibody or antibody fragment to be used in such patient-specific immunotherapy can be used neat, or as a component of bifunctional agents consisting of the antibody-mediated targeting end linked to either toxins, enzymes or radioisotopes to confer a more effective toxic payload. To construct a bifunctional immunotherapeutic consisting of a toxin conjugated antibody, one could fuse at the gene level the gene sequence encoding the antibody or antibody fragment to one encoding a toxin or enzyme. The translated protein would consist of the target-binding heavy and light variable regions of the Ig, and the toxin-or enzyme-linked antibody constant regions. Upon establishing highly specific targeting of the combined moiety by virtue of the antibody-mediated reaction, the toxin or enzyme would act on the surface of the target cell, or be internalized to destroy the cell from within, depending on its characteristics and mode of action. Toxins that could be used in this mode of therapy include cholera, diphtheria, ricin, etc. Alternatively, post Ig synthesis a radioisotope or toxin or enzyme could be chemically conjugated to the Ig to produce essentially the same bifunctional agent. Radioisotopes that can be used in this therapy include Iodine, Yttrium, etc. Both toxins and radioisotopes with medical utility and approved for use by the regulatory agencies are known to those skilled in the art. In either case, the neat Ig or bifunctional Ig or Ig fragment would be administered to the patient with the defined affliction, probably by intravenous infusion, so that the drug could target and destroy very specifically only the pathogen or malignant cell population, while sparing the non-target or healthy cells and tissues.

While smaller antibody fragments have an advantage over whole Ig proteins in penetration and permeability, one of their disadvantages is their more rapid removal from circulation. Because an antibody's ability to find and bind to a target is a function of dose, time in circulation, and binding affinity of the Ig to its target, a longer residence time is desirable for achieving a lower dose (lower cost, lower potential toxicity) and higher efficacy. There are formulations, alterations and modifications that could be used to increase the Ig's circulating half-life. For example, polyethylene glycol has been used to extend the half-life of therapeutic proteins such as interferons (interferon alpha 2a, eg. PEG-Intron [Schering-Plough], Pegasys, [Roche]), and enzymes (L-asparaginase, eg. ONCASPAR; adenosine deaminase, eg. ADAGEN [Enzon Pharmaceuticals]), as well as synthetic drugs. PEG acts as an inert coat to protect drugs, especially proteins, from immune-mediated and other natural removal mechanisms. The Fab and scFv versions of patient-specific antibody fragments could be PEGylated as well, to impart longer circulating half-lives, possibly lowering the required dose (potentially lowering the cost of the therapy), and making administration less frequent, while maintaining the advantages of capillary and tissue penetration of the Ig drug enabled by the lower MW and lower size of the fragments relative to the whole Ig. PEGylation is accomplished by chemically grafting PEG chains, which would be linear or branched, permanent or releasable, and of various MW, onto the Ig, Ig fragment, or Ig-fragment bifunctional conjugate. The chemistry for effecting PEGylation has been described and is well known to those skilled in the art.

Conclusions

The following are representative of the structures and methods represented by this invention.

1. An artificial preproprotein, comprising four peptide sequences:

(a) a signal peptide sequence;

(b) a first peptide sequence of interest attached to the c-terminus of the signal peptide sequence;

(c) a propeptide sequence attached to the c-terminus of the first peptide sequence of interest; and

(d) a second peptide of interest attached to the c-terminus of the propeptide sequence

wherein the propeptide sequence is not naturally associated with either the first or the second peptide of interest.

2. The artificial preproprotein of conclusion 1 that comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody, but the first peptide is different from the second peptide.

3. The artificial preproprotein of conclusion 1 that comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody.

4. The artificial preproprotein of conclusion 3 wherein the first peptide and the second peptide are both heavy chain peptides.

5. The artificial preproprotein of conclusion 3 wherein the first peptide is a light chain of the antibody.

6. The artificial preproprotein of conclusion 1 that comprises a Fab fragment light chain peptide and an Fab fragment heavy chain peptide, wherein the first peptide is either a heavy chain of the Fab fragment or a light chain of the Fab fragment, and wherein the second peptide is either a heavy chain of the Fab fragment or a light chain of the Fab fragment, but the first peptide, but the first peptide is different from the second peptide.

7. The artificial preproprotein of conclusion 1 that comprises a Fab fragment light chain peptide and an Fab fragment heavy chain peptide, wherein the first peptide is either a heavy chain of the Fab fragment or a light chain of the Fab fragment, and wherein the second peptide is either a heavy chain of the Fab fragment or a light chain of the Fab fragment.

8. The artificial preproprotein of conclusion 7 wherein the first peptide and the second peptide are both heavy chain peptides.

9. The artificial preproprotein of conclusion 7 wherein the first peptide and the second peptide are both light chain peptides.

10. The artificial preproprotein of conclusion 1 that comprises a light chain peptide and a heavy chain peptide of a Fab fragment derivative or an antibody derivative, wherein the first peptide is either a heavy chain of the Fab fragment or Antibody derivative or a light chain of the Fab fragment or Antibody derivative, and wherein the second peptide is either a heavy chain of the Fab fragment or Antibody derivative or a light chain of the Fab fragment or Antibody derivative but the first peptide is different from the second peptide.

11. The artificial preproprotein of conclusion 1 that comprises a light chain peptide and a heavy chain peptide of a Fab fragment derivative or an antibody derivative, wherein the first peptide is either a heavy chain of the Fab fragment or antibody derivative or a light chain of the Fab fragment or antibody derivative, and wherein the second peptide is either a heavy chain of the Fab fragment or antibody derivative or a light chain of the Fab fragment or antibody derivative.

12. The artificial preproprotein of conclusion 11 wherein the first peptide and the second peptide are both heavy chain peptides.

13. The artificial preproprotein of conclusion 11 wherein the first peptide and the second peptide are both light chain peptides.

14. An artificial polynucleotide, comprising four nucleotide sequences:

a first nucleotide sequence that encodes a signal peptide sequence;

a second nucleotide sequence that encodes a first peptide of interest, second nucleotide sequence being connected to the 3′ terminus of the first nucleotide sequence;

a third nucleotide sequence that encodes a propeptide, third nucleotide sequence being connected to the 3′ terminus of the second nucleotide sequence; and

a fourth nucleotide sequence that encodes a second peptide of interest, fourth nucleotide sequence being connected to the 3′ terminus of the third nucleotide sequence.

15. The artificial polynucleotide of conclusion 14 that encodes a polypeptide that comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody, but the first peptide is different from the second peptide.

16. The artificial polynucleotide of conclusion 14 that encodes a polypeptide that comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody.

17. The artificial polynucleotide of conclusion 16 wherein the first peptide and the second peptide are both heavy chain peptides.

18. The artificial polynucleotide of conclusion 16 wherein the first peptide is a light chain of the antibody.

19. The artificial polynucleotide of conclusion 14 that encodes a polypeptide that comprises a Fab fragment light chain peptide and an Fab fragment heavy chain peptide, wherein the first peptide is either a heavy chain of the Fab fragment or a light chain of the Fab fragment, and wherein the second peptide is either a heavy chain of the Fab fragment or a light chain of the Fab fragment, but the first peptide, but the first peptide is different from the second peptide.

20. The artificial polynucleotide of conclusion 14 that encodes a polypeptide that comprises a Fab fragment light chain peptide and an Fab fragment heavy chain peptide, wherein the first peptide is either a heavy chain of the Fab fragment or a light chain of the Fab fragment, and wherein the second peptide is either a heavy chain of the Fab fragment or a light chain of the Fab fragment.

21. The artificial polynucleotide of conclusion 20 wherein the first peptide and the second peptide are both heavy chain peptides.

22. The artificial polynucleotide of conclusion 20 wherein the first peptide and the second peptide are both light chain peptides.

23. The artificial polynucleotide of conclusion 14 that encodes a polypeptide that comprises a light chain peptide and a heavy chain peptide of a Fab fragment derivative or an antibody derivative, wherein the first peptide is either a heavy chain of the Fab fragment derivative or antibody derivative or a light chain of the Fab fragment or antibody derivative, and wherein the second peptide is either a heavy chain of the Fab fragment or antibody derivative or a light chain of the Fab fragment or antibody derivative but the first peptide is different from the second peptide.

24. The artificial polynucleotide of conclusion 14 that encodes a polypeptide that comprises a light chain peptide and a heavy chain peptide of a Fab fragment derivative or an antibody derivative, wherein the first peptide is either a heavy chain of the Fab fragment derivative or antibody derivative or a light chain of the Fab fragment or antibody derivative.

25. The artificial polynucleotide of conclusion 24 wherein the first peptide and the second peptide are both heavy chain peptides.

26. The artificial polynucleotide of conclusion 24 wherein the first peptide and the second peptide are both light chain peptides.

27. A method of making an artificial polynucleotide of conclusion 14, comprising:

providing a first, a second, a third and a fourth nucleotide sequence that encode a signal peptide sequence, a first peptide of interest, a propeptide and a second peptide of interest respectively;

connecting the 3′ terminus of the first nucleotide sequence to the 5′ terminus of the second nucleotide sequence;

connecting the 3′ terminus of the second nucleotide sequence to the 5′ terminus of the third nucleotide sequence; and

connecting the 3′ terminus of the third nucleotide sequence to the 5′ terminus of the fourth nucleotide sequence, wherein the nucleotide sequence that encodes a first peptide of interest can be the same as or different from the nucleotide sequence that encodes a second peptide of interest.

28. The method of conclusion 27 wherein the artificial polynucleotide encodes a polypeptide that comprises an antibody light chain peptide and an antibody heavy chain peptide, wherein the second nucleotide sequence encodes either a heavy chain of the antibody or a light chain of the antibody, and wherein the fourth nucleotide sequence encodes either a heavy chain of the antibody or a light chain of the antibody, but the second nucleotide sequence is different from the fourth nucleotide sequence.

29. The method of conclusion 27 wherein the artificial polynucleotide encodes a polypeptide that comprises an antibody light chain peptide and an antibody heavy chain peptide, wherein the second nucleotide sequence encodes either a heavy chain of the antibody or a light chain of the antibody, and wherein the fourth nucleotide sequence encodes either a heavy chain of the antibody or a light chain of the antibody.

30. The method of conclusion 29 wherein the second nucleotide sequence and the fourth nucleotide sequence both encode a heavy chain polypeptide.

31. The method of conclusion 29 wherein the second nucleotide sequence and the fourth nucleotide sequence both encode a light chain polypeptide.

32. The method of conclusion 27 wherein the artificial polynucleotide encodes a polypeptide that comprises a Fab light chain peptide and an antibody heavy chain peptide, wherein the second nucleotide sequence encodes either a heavy chain of the Fab fragment or a light chain of the Fab fragment, and wherein the fourth nucleotide sequence encodes either a heavy chain of the Fab fragment or a light chain of the Fab fragment, but the second nucleotide sequence is different from the fourth nucleotide sequence

33. The method of conclusion 27 wherein the artificial polynucleotide encodes a polypeptide that comprises a Fab light chain peptide and an antibody heavy chain peptide, wherein the second nucleotide sequence encodes either a heavy chain of the Fab fragment or a light chain of the Fab fragment, and wherein the fourth nucleotide sequence encodes either a heavy chain of the Fab fragment or a light chain of the Fab fragment.

34. The method of conclusion 33 wherein the second nucleotide sequence and the fourth nucleotide sequence both encode a heavy chain polypeptide.

35. The method of conclusion 33 wherein the second nucleotide sequence and the fourth nucleotide sequence both encode a light chain polypeptide.

36. The method of conclusion 27 wherein the artificial polynucleotide encodes a polypeptide that comprises a light chain peptide and a heavy chain peptide of a Fab fragment derivative or an antibody derivative, wherein the second nucleotide sequence encodes either a heavy chain of the Fab fragment or Antibody derivative or a light chain of the Fab fragment or Antibody derivative, and wherein the fourth nucleotide sequence encodes either a heavy chain of the Fab fragment or Antibody derivative or a light chain of the Fab fragment or Antibody derivative but the second nucleotide sequence is different from the fourth nucleotide sequence

37. The method of conclusion 27 wherein the artificial polynucleotide encodes a polypeptide that comprises a light chain peptide and a heavy chain peptide of a Fab fragment derivative or an antibody derivative, wherein the second nucleotide sequence encodes either a heavy chain of the Fab fragment or Antibody derivative or a light chain of the Fab fragment or Antibody derivative, and wherein the fourth nucleotide sequence encodes either a heavy chain of the Fab fragment or Antibody derivative or a light chain of the Fab fragment or Antibody derivative.

38, The method of conclusion 37 wherein the second nucleotide sequence and the fourth nucleotide sequence both are derived from a nucleotide sequence that encodes a heavy chain peptide.

39. The method of conclusion 37 wherein the second nucleotide sequence and the fourth nucleotide sequence both are derived from a nucleotide sequence that encodes a light chain peptide.

40. A method of making an artificial preproprotein, comprising: making an artificial polynucleotide that encodes the preproprotein; and expressing the artificial polynucleotide in a host organism whereby the preproprotein is made.

41. A method of making a multimeric protein, comprising:

providing a first, a second, a third and a fourth nucleotide sequence that encode a signal peptide sequence, a first peptide of interest, a propeptide and a second peptide of interest respectively;

connecting the 3′ terminus of the first nucleotide sequence to the 5′ terminus of the second nucleotide sequence;

connecting the 3′ terminus of the second nucleotide sequence to the 5′ terminus of the third nucleotide sequence; and

connecting the 3′ terminus of the third nucleotide sequence to the 5′ terminus of the fourth nucleotide sequence, so that an artificial polynucleotide results and is comprised of the four nucleotide sequences, and wherein the nucleotide sequence that encodes a first peptide of interest can be the same as or different from the nucleotide sequence that encodes a second peptide of interest;

introducing the resulting artificial polynucleotide into a host organism by transfection, or by stable transformation;

allowing the artificial polynucleotide to be expressed in the host organism whereby a preproprotein is made;

allowing the preproprotein to be processed into a mature polypeptide.

42. The method of conclusion 41 further comprising allowing two copies of the mature polypeptide to bond to form a mature multimeric protein.

43. The method of conclusion 41 wherein the multimeric protein is an antibody or a Fab fragment or a derivative of either the antibody or the Fab fragment.

44. A vector encoding an artificial preproprotein, comprising:

a nucleotide sequence necessary for replication of the vector nucleotides and proteins and

the artificial polynucleotide of conclusion 14 inserted into the vector.

45. The vector of conclusion 44 that is a plasmid or a viral vector.

46. The vector of conclusion 44 that is capable of being reproduced in a microorganism.

47. A transiently transformed cell, comprising:

A vector encoding an artificial preproprotein, comprising:

a nucleotide sequence necessary for replication of the vector nucleotides and for expression of proteins;

an artificial polynucleotide encoding an artificial preproprotein of claim 14 inserted into the vector,

a promoter capable of directing expression of the artificial preproprotein; and

the artificial preproprotein encoded by the artificial polynucleotide.

48. The cell of conclusion 47 wherein the artificial preproprotein comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody, but the first peptide is different from the second peptide.

49. The cell of conclusion 47, the cell further comprising a mature multimeric protein made from two copies of the artificial preproprotein.

50. An organism comprising a plurality of cells according to conclusion 47.

51. A plant, an animal, a fungus, or an algae organism according to conclusion 49 or 50 wherein the organism is a plant, an animal a fungus or an algae.

52. A plant cell, an animal cell, a fungus cell, an algae cell or a single celled organism according to conclusion 47.

53. An organism comprising at least one cell according to conclusion 47 wherein the multimeric protein is secreted into the interstitial spaces or fluids of the organism.

54. An organism according to conclusion 49 wherein the multimeric protein is secreted into the circulatory or excreatatory system of the organism.

55. A transgenic cell, comprising:

(a) an artificial polynucleotide of conclusion 14 stably incorporated onto a chromosome,

(b) optionally a promoter capable of directing expression of the artificial preproprotein; and

(c) The artificial preproprotein encoded by the artificial polynucleotide.

56. The cell of conclusion 55 wherein The artificial preproprotein comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody, but the first peptide is different from the second peptide.

57. An organism comprising the cell of conclusion 55, the cell further comprising a mature multimeric protein made from two copies of the artificial preproprotein.

58. An organism comprising a plurality of cells according to conclusion 57.

59. A plant, an animal, a fungus, or an algae organism according to conclusion 57 or 58 wherein the organism is a plant, an animal a fungus or an algae.

60. A plant cell, an animal cell, a fungus cell, an algae cell or a single celled organism according to conclusion 55.

61. An organism comprising at least one cell according to conclusion 55 wherein the multimeric protein is secreted into the interstitial spaces or body fluids of the organism.

62. An organism according to conclusion 49 wherein the multimeric protein is secreted into the circulatory or excretatory system of the organism.

63. A transgenic or transiently transformed organism containing or incorporating the artificial preproprotein of conclusion 1.

64. A transgenic or transiently transformed plant, comprising:

(a) plant cells containing an artificial polynucleotide sequence encoding an artificial preproprotein that artificial preproprotein comprises a) a signal peptide sequence, b) an immunoglobulin heavy chain or light chain peptide, c) a propeptide, and d) an immunoglobulin heavy chain or light chain peptide, wherein the heavy chain can be in either the b or the d position on the preproprotein, and the light chain will be on the other position, wherein The artificial preproprotein contains a signal peptide sequence signal peptide sequence forming a secretion signal; and

(b) containing immunoglobulin molecules encoded by said artificial polynucleotide sequence, wherein said signal peptide sequence signal peptide sequence is cleaved from said artificial preproprotein by proteolytic processing, and wherein said propeptide is cleaved from the heavy chain and the light chain following proper folding of the remaining polypeptide.

65. The plant of conclusion 64 wherein the signal peptide sequences is a heterologous signal peptide sequence.

66. The plant of conclusion 64 wherein the polynucleotide sequence encodes a mammalian immunoglobulin.

67. The plant of conclusion 64 wherein the immunoglobulin is an immunoglobulin superfamily molecule.

68. The plant of conclusion 64 that is a dicotyledonous plant.

69. The plant of conclusion 64 that is a monocotyledonous plant. (corn etc.)

70. The plant of conclusion 64, that is a Nicotiana plant.

71. The plant of conclusion 64, wherein said polynucleotide sequence encoding the preproprotein is present on a single vector.

72. A method for making a transgenic plant capable of producing immunoglobulin molecules, comprising:

(a) introducing into the genome of a member of a plant species an artificial polynucleotide sequence encoding a preproprotein that preproprotein comprises (i) a signal peptide sequence, (ii) an immunoglobulin heavy chain or light chain peptide, (iii) a propeptide, and (iv) an immunoglobulin heavy chain or light chain peptide, wherein the heavy chain can be in either the b or the d position on the preproprotein, and the light chain will be on the other position; and

(b) allowing stable transformation to occur to produce a transformant.

73. The method of conclusion 72 wherein the signal peptide sequence is a heterologous signal peptide sequence.

74. The method of conclusion 72 wherein said first and second nucleotide sequences are introduced via the same vector.

75. The plant of conclusion 64, wherein at least some of said immunoglobulin molecules are present within the cell wall of said plant cells.

76. The plant of conclusion 64, wherein said immunoglobulin molecules are trafficked through the golgi of said plant cells.

77. The plant of conclusion 64, wherein said immunoglobulin molecules are selected from the group consisting of IgA, IgD, IgE, IgG, or IgM isotypes.

78. The plant of conclusion 64, wherein said immunoglobulin molecules comprise the IgG isotype.

79. The plant of conclusion 64, wherein said immunoglobulin molecules comprise the IgA isotype.

80. The transgenic plant of conclusion 64 wherein The artificial preproprotein further comprises a promoter directing expression of said artificial polynucleotide.

81. The plant of conclusion 64, wherein substantially all of the heavy- and light-chain peptides are assembled to form immunoglobulin molecules within said plant cell.

82. The transgenic plant of conclusion 80 the promoter is a constitutive promoter.

83. An artificial proprotein, comprising three peptide sequences:

(a) a first peptide sequence of interest;

(b) a propeptide sequence attached to the c-terminus of the first peptide sequence of interest; and

(c) a second peptide of interest attached to the c-terminus of the propeptide sequence.

84. The artificial proprotein of conclusion 83 further comprising a signal peptide sequence attached to the N-terminus of the first peptide sequence of interest.

85. A process for producing an immunoglobulin molecule or an immunologically functional immunoglobulin fragment comprising at least the variable domains of the immunoglobulin heavy and light chains, in a single host cell, comprising the steps of:

(a) transforming said single host cell with a single DNA sequence encoding at least the variable domain of the immunoglobulin heavy chain, a propeptide and at least the variable domain of the immunoglobulin light chain, and

(b) expressing said single DNA sequence so that said immunoglobulin heavy and light chains are produced as a single propeptide molecule in said transformed single host cell.

86. The process according to conclusion 85 wherein said single DNA sequence is present in different vectors.

87. The process according to conclusion 85 wherein said single DNA sequence is present in a single vector.

88. A process according to conclusion 87 wherein the vector is a plasmid.

89. The process according to conclusion 88 wherein the plasmid is pBR322 or a derivative thereof.

90. The process according to conclusion 85 wherein the host cell is a bacterium or yeast.

91 The process according to conclusion 90 wherein the host cell is E. coli or S. cerevisiae.

92. A process according to conclusion 85 wherein the immunoglobulin heavy and light chains are expressed in the host cell and secreted therefrom as an immunologically functional immunoglobulin molecule or immunoglobulin fragment.

93. A process according to conclusion 85 wherein the immunoglobulin heavy and light chains are produced in insoluble form and are solubilized and allowed to refold in solution to form an immunologically functional immunoglobulin molecule or immunoglobulin fragment.

94. A process according to conclusion 85 wherein the DNA sequence codes for the complete immunoglobulin heavy and light chains.

95. The process according to conclusion 85 wherein said single DNA sequence further encodes at least one constant domain, wherein the constant domain is derived from the same source as the variable domain to which it is attached.

96. The process according to conclusion 85 wherein said single DNA sequence further encodes at least one constant domain, wherein the constant domain is derived from a species or class different from that from which the variable domain to which it is attached is derived.

97. The process according to conclusion 85 wherein said single DNA sequence is derived from one or more monoclonal antibody-producing hybridomas.

98. A vector comprising a single DNA sequence encoding at least a variable domain of an immunoglobulin heavy chain and at least a variable domain of an immunoglobulin light chain wherein said single DNA sequence is located in said vector at a single insertion site.

99. A vector according to conclusion 98 that is a plasmid.

100. A host cell transformed with a vector according to conclusion 98.

101. A transformed host cell comprising at least two vectors, at least one of said vectors comprising a single DNA sequence encoding at least a variable domain of an immunoglobulin heavy chain and at least the variable domain of an immunoglobulin light chain.

102. The process of conclusion 85 wherein the host cell is a mammalian cell.

103. The transformed host cell of conclusion 101 wherein the host cell is a mammalian cell.

104. A method comprising:

(a) preparing a DNA sequence consisting essentially of DNA encoding an immunoglobulin consisting of an immunoglobulin heavy chain and light chain or Fab region, said immunoglobulin having specificity for a particular known antigen, wherein the DNA sequence incorporates an artificial polynucleotide encoding a proprotein which consists of at least a variable domain of an immunoglobulin heavy chain, a cleavable propeptide, and at least the variable domain of an immunoglobulin light chain;

(b) inserting the DNA sequence of step a) into a replicable expression vector operably linked to a suitable promoter;

(c) transforming a prokaryotic or eukaryotic microbial host cell culture with the vector of step(b);

(d) culturing the host cell; and

(e) recovering the immunoglobulin from the host cell culture, said immunoglobulin being capable of binding to a known antigen.

105. The method of conclusion 104 wherein the heavy and light chain are the heavy and light chains of anti-CEA antibody.

106. The method of conclusion 104 wherein the heavy chain is of the gamma family.

107. The method of conclusion 104 wherein the light chain is of the kappa family.

108. The method of conclusion 104 wherein the vector contains DNA encoding both a heavy chain and a light chain.

109. The method of conclusion 104 wherein the host cell is E. coli or yeast.

110. The method of conclusion 109 wherein the heavy chain and light chains or Fab region are deposited within the cells as insoluble particles.

111. The method of conclusion 109 wherein the proprotein is deposited within the cells as insoluble particles.

112. The method of conclusion 110 wherein the proprotein is recovered from the particles by cell lysis followed by solubilization in denaturant.

113. The method of conclusion 104 wherein the proprotein is secreted into the medium.

114. The method of conclusion 104 wherein the host cell is a gram negative bacterium and the proprotein is secreted into the periplasmic space of the host cell bacterium.

115. The method of conclusion 104 further comprising recovering both heavy and light chain and reconstituting light chain and heavy chain to form an immunoglobulin having specific affinity for a particular known antigen.

116. The insoluble particles of heavy chain and light chains or Fab region produced by the method of conclusion 110.

117. A process for producing an immunoglobulin molecule or an immunologically functional immunoglobulin fragment comprising at least the variable domains of the immunoglobulin heavy and light chains, in a single host cell, comprising:

(a) expressing a single DNA sequence encoding at least the variable domain of the immunoglobulin heavy chain and at least the variable domain of the immunoglobulin light chain so that said immunoglobulin heavy and light chains are produced as a single proprotein molecule in said single host cell transformed with said single DNA sequence.

118. The process of conclusion 92, further comprising the step of attaching the immunoglobulin molecule or immunoglobulin fragment to a label or drug.

119. The process of conclusion 93, further comprising the step of attaching the immunoglobulin molecule or immunoglobulin fragment to a label or drug.

120. The process of conclusion 117, further comprising the step of attaching the immunoglobulin molecule or immunoglobulin fragment to a label or drug.

121. A multimeric protein encoded by an artificial polynucleotide according to conclusion 14, the multimeric protein selected from the group consisting of hemoglobin (α₂β₂), IL-12, TCR, MHC class II heterodimer (α,β), CD8 heterodimer (α,β), CD3 (εδ), CD3 (εγ), CD22(α,β), CD41(GPIIba CD61) Janus kinase(JAK), JAK and STAT (signal transducers and activators of transcription) heterodimers, IgM heavy chain with I chain, or VpreB and lambda 5 (I chain), Igβ and Igα, Integrins , T-cell integrin LFA-1 (α_{L}β₂), CD152(CTLA-4), IL-2 receptor(heterotrimer) IL-2R(αβγc), IL-15(αβγ), Rhematopoietin receptor family (IL-3R, GM-CSFR are a few), TNF-62 (LT-α and LT-β), IL12R(β1β2), IgM (H₂L₂) with transgenic J chain, IgA (H₂L₂) with transgenic J chain, MHC class I (α and β₂-microglobulin), HLA-DM(αβ),H-2M(αβ), E.coli DNA polymerase III, insulin receptor(IR) (α₂β₂), IGF-1 receptor(α₂β₂), G proteins heterotrimers (αβγ),adrenergic receptor, retinoic acid receptor (RAR) (αβ), oestrogen receptor(αβ),myocyte enhancer factors 2 (MEF2) family, c-fos and JunD, yeast RNAPII Rpb3/Rpb11 heterodimer, calpain, importin alpha2/beta heterodimer, DNA-dependent protein kinase (DNA-PKcs, and Ku70 and Ku80), Ku70 and Ku80 heterodimer, Hepatopoietin (HPO) and HPO23 heterodimer, leukocyte function associated antigen-1 molecule (LFA-1) CD11 a (alphaL) and CD18 (beta2) integrin subunit heterodimer, liver X receptor (LXR)/retinoid X receptor (RXR) heterodimer, eukaryotic structural maintenance of chromosome (SMC) proteins, human mismatch repair (MMR) heterodimers, rBAT-b(0,+)AT heterodimer, retinoid X alpha (RXRalpha) and peroxisome proliferator-activated receptor alpha (PPARalpha) heterodimer, thyroid hormone receptor (TR)/RXR heterodimer, peroxisome proliferator activated receptor/RXR, Nurr1 orphan nuclear receptor/RXR heterodimer, calcineurin, Collapsin response mediator protein-2 and tubulin heterodimer, CD94/NKG2A heterodimer, IkappaB kinase complex, human immunodeficiency virus reverse transcriptase (RT) heterodimer, CD98 complex, B cell antigen receptor with the membrane-bound immunoglobulin molecule (mIg) and the Ig-alpha/Ig-beta heterodimer, class IA phosphoinositide 3-kinase, hypoxia inducible factor 1.

122. The transgenic plant of conclusion 80 the promoter is an inducible promoter.

123. A multimeric protein, comprising first and second peptides, the first peptide comprising a non-native amino acid pair at the P1 and P2 positions of the carboxy terminus.

124. A multimeric protein according to conclusion 1 wherein the P2 position is occupied by Lys, Pro, or Arg.

125. A multimeric protein according to conclusion 1 wherein the P1 position is occupied by Lys, Pro, or Arg.

126. A multimeric protein derived from a multimeric protein, comprising a first and second peptides, the first peptide comprising a non-native amino acid pair at the P1 and P2 positions of the carboxy terminus.

Deposit Information

cDNAs were then deposited under the terms of the Budapest Treaty with the American Type Culture Collection, 10801 University Blvd., Manassas, Va. 20110-2209, USA (ATCC) as shown:

Plasmid DNA: p5PNCAP is Patent Deposit PTA-4742 Deposited Oct. 3, 2002

Plasmid DNA: p1177MP5 is Patent Deposit PTA-4743 Deposited Oct. 3, 2002

Plasmid DNA: p1324-MBP is Patent Deposit PTA-4744 Deposited Oct. 3, 2002

Plasmid DNA: pLSBC1798 is Patent Deposit ______ Deposited Oct. 2, 2003

Plasmid DNA: pLSBC2634 is Patent Deposit ______ Deposited Oct. 2, 2003

Plasmid DNA: Hu Fab A9 is Patent Deposit ______ Deposited Oct. 2, 2003

Plasmid DNA: Hu Fab D5 is Patent Deposit ______ Deposited Oct. 2, 2003

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit or 5 years after the last request, whichever is later. The assignee of the present application has agreed that if a culture of the materials on deposit should be found non viable or be lost or destroyed, the materials will be promptly replaced on notification with another of the same. Availability of the deposited material is not to be construed as a license to practice the invention in contravention of the rights granted under the authority of any government in accordance with its patent laws, or as a license to use the deposited material for research.

Accordingly, the present invention has been described with some degree of particularity directed to the preferred embodiment of the present invention. It should be appreciated, though, that the present invention is defined by the following claims construed in light of the prior art so that modifications or changes may be made to the preferred embodiment of the present invention without departing from the inventive concepts contained herein.

What is claimed is:

1. An artificial proprotein, comprising three peptide sequences:
(a) a first peptide sequence of interest;
(b) a propeptide sequence attached to the C-terminus of the first peptide sequence of interest; and
(c) a second peptide of interest attached to the C-terminus of the propeptide sequence.

2. The artificial proprotein of claim 1, further comprising a signal peptide sequence attached to the N-terminus of the first peptide sequence of interest.

3. The artificial proprotein of claim 1 that comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody.

4. The artificial proprotein of claim 1 that comprises an antibody light chain peptide and a Fd heavy chain peptide, wherein the first peptide is either a heavy chain or an antibody light chain, and wherein the second peptide is either a Fd or an antibody light chain.

5. The artificial proprotein of claim 1 that comprises a light chain peptide and a heavy chain peptide of a Fab fragment derivative or an antibody derivative, wherein the first peptide is either a heavy chain of the Fab fragment or antibody derivative or a light chain of the Fab fragment or antibody derivative, and wherein the second peptide is either a heavy chain of the Fab fragment or antibody derivative or a light chain of the Fab fragment or antibody derivative.

6. An artificial polynucleotide, comprising four nucleotide sequences:
(a) a first nucleotide sequence that encodes a signal peptide sequence;
(b) a second nucleotide sequence that encodes a first peptide of interest, second nucleotide sequence being connected to the 3′ terminus of the first nucleotide sequence;
(c) a third nucleotide sequence that encodes a propeptide, third nucleotide sequence being connected to the 3′ terminus of the second nucleotide sequence; and
(d) a fourth nucleotide sequence that encodes a second peptide of interest, fourth nucleotide sequence being connected to the 3′ terminus of the third nucleotide sequence.

7. The artificial polynucleotide of claim 6 that encodes a polypeptide that comprises an antibody light chain peptide and an antibody heavy chain peptide, wherein the first peptide is either a heavy chain of the antibody or a light chain of the antibody, and wherein the second peptide is either a heavy chain of the antibody or a light chain of the antibody.

8. The artificial polynucleotide of claim 6 that encodes a polypeptide that comprises a Fab fragment light chain peptide and a Fab fragment heavy chain peptide, wherein the first peptide is either a heavy chain of the Fab fragment or a light chain of the Fab fragment, and wherein the second peptide is either a heavy chain of the Fab fragment or a light chain of the Fab fragment.

9. The artificial polynucleotide of claim 6 that encodes a polypeptide that comprises a light chain peptide and a heavy chain peptide of a Fab fragment derivative or an antibody derivative, wherein the first peptide is either a heavy chain of the Fab fragment derivative or antibody derivative or a light chain of the Fab fragment or antibody derivative.

10. A method of making the artificial polynucleotide of claim 6, comprising:
(a) providing a first, a second, a third and a fourth nucleotide sequence that encode a signal peptide sequence, a first peptide of interest, a propeptide and a second peptide of interest, respectively,
(b) connecting the 3′ terminus of the first nucleotide sequence to the 5′ terminus of the second nucleotide sequence;
(c) connecting the 3′ terminus of the second nucleotide sequence to the 5′ terminus of the third nucleotide sequence; and
(d) connecting the 3′ terminus of the third nucleotide sequence to the 5′ terminus of the fourth nucleotide sequence, wherein the nucleotide sequence that encodes a first peptide of interest can be the same as or different from the nucleotide sequence that encodes a second peptide of interest.

11. The method of claim 10 wherein the artificial polynucleotide encodes a polypeptide that comprises an antibody light chain peptide and an antibody heavy chain peptide, wherein the second nucleotide sequence encodes either a heavy chain of the antibody or a light chain of the antibody, and wherein the fourth nucleotide'sequence encodes either a heavy chain of the antibody or a light chain of the antibody.

12. The method of claim 10 wherein the artificial polynucleotide encodes a polypeptide that comprises a Fab light chain peptide and a Fab heavy chain peptide, wherein the second nucleotide sequence encodes either a heavy chain of the Fab fragment or a light chain of the Fab fragment, and wherein the fourth nucleotide sequence encodes either a heavy chain of the Fab fragment or a light chain of the Fab fragment.

13. The method of claim 10 wherein the artificial polynucleotide encodes a polypeptide that comprises a light chain peptide and a heavy chain peptide of a Fab fragment derivative or an antibody derivative, wherein the second nucleotide sequence encodes either a heavy chain of the Fab fragment or antibody derivative or a light chain of the Fab fragment or antibody derivative, and wherein the fourth nucleotide sequence encodes either a heavy chain of the Fab fragment or antibody derivative or a light chain of the Fab fragment or antibody derivative.

14. A method of making an artificial preproprotein, comprising:
(a) making an artificial polynucleotide that encodes the preproprotein; and
(b) expressing the artificial polynucleotide in a host organism whereby the preproprotein is made.

15. A method of making a multimeric protein, comprising:
(a) providing a first, a second, a third and a fourth nucleotide sequence that encode a signal peptide sequence, a first peptide of interest, a propeptide and a second peptide of interest, respectively;
(b) connecting the 3′ terminus of the first nucleotide sequence to the 5′ terminus of the second nucleotide sequence;
(c) connecting the 3′ terminus of the second nucleotide sequence to the 5′ terminus of the third nucleotide sequence; and
(d) connecting the 3′ terminus of the third nucleotide sequence to the 5′ terminus of the fourth nucleotide sequence, so that an artificial polynucleotide results and is comprised of the four nucleotide sequences, and wherein the nucleotide sequence that encodes a first peptide of interest can be the same as or different from the nucleotide sequence that encodes a second peptide of interest;
(i) introducing the resulting artificial polynucleotide into a host organism by transfection, or by stable transformation;
(ii) allowing the artificial polynucleotide to be expressed in the host organism whereby a preproprotein is made;
(iii) allowing the preproprotein to be processed into a mature polypeptide.

16. The method of claim 15 further comprising allowing two copies of the mature polypeptide to bond to form a mature multimeric protein.

17. The method of claim 15 wherein the multimeric protein is an antibody or a Fab fragment or a derivative of either the antibody or the Fab fragment.

18. A vector encoding an artificial preproprotein, comprising:
(a) a nucleotide sequence necessary for replication of the vector nucleotides and proteins; and
(b) the artificial polynucleotide of claim 6 inserted into the vector.

19. A method for making a transgenic plant capable of producing immunoglobulin molecules, comprising:
(a) introducing into the genome of a member of a plant species an artificial polynucleotide sequence encoding a preproprotein wherein the preproprotein comprises a) a signal peptide sequence, b) an immunoglobulin heavy chain or light chain peptide, c) a propeptide, and d) an immunoglobulin heavy chain or light chain peptide, wherein the heavy chain can be in either the b or the d position on the preproprotein, and the light chain will be on the other position; and
(b) allowing stable transformation to occur to produce a transformant.

20. A vector comprising a single DNA sequence encoding at least a variable domain of an immunoglobulin heavy chain and at least a variable domain of an immunoglobulin light chain wherein said single DNA sequence is located in said vector at a single insertion site.

21. A method comprising:
(a) preparing a DNA sequence consisting essentially of DNA encoding an immunoglobulin consisting of an immunoglobulin heavy chain and light chain or Fab region, said immunoglobulin having specificity for a particular known antigen, wherein the DNA sequence incorporates an artificial polynucleotide encoding a proprotein which consists of at least a variable domain of an immunoglobulin heavy chain, a cleavable propeptide, and at least the variable domain of an immunoglobulin light chain;
(b) inserting the DNA sequence of step a) into a replicable expression vector operably linked to a suitable promoter;
(c) transforming a prokaryotic or eukaryotic microbial host cell culture with the vector of step b);
(d) culturing the host cell; and
(e) recovering the immunoglobulin from the host cell culture, said immunoglobulin being capable of binding to a known antigen.

22. A process for producing an immunoglobulin molecule or an immunologically functional immunoglobulin fragment comprising at least the variable domains of the immunoglobulin heavy and light chains, in a single host cell, comprising:
expressing a single DNA sequence encoding at least the variable domain of the immunoglobulin heavy chain and at least the variable domain of the immunoglobulin light chain so that said immunoglobulin heavy and light chains are produced as a single proprotein molecule in said single host cell transformed with said single DNA sequence.

23. A multimeric protein, comprising first and second peptides, the first peptide comprising a non-native amino acid pair at the P1 and P2 positions of the carboxy terminus.

24. A multimeric protein according to claim 23 wherein the P2 position is occupied by Lys, Pro, or Arg.

25. A multimeric protein according to claim 23 wherein the P1 position is occupied by Lys, Pro, or Arg.

26. A multimeric protein derived from the multimeric protein of claim 23, comprising a first and second peptides, the first peptide comprising a non-native amino acid pair at the P1 and P2 positions of the carboxy terminus.
